SEQUENCE LISTING (531 sequences)

SEQ ID NO: 1 - protein, 112 aa, synthetic construct
  REGION 1..112: note = Description of Artificial Sequence: Syntheticpolypeptide
DIVMTQNPLSSPVTLGQPASISCRSSQSLVHSDGNTYLSWLQQRPGQSPRLLIYKISNRFSGVPDRFSGSGAGTDFTLKISRVEAEDVGVYYCMQATQFPLTIGGGSKVEIK

SEQ ID NO: 2 - protein, 118 aa, synthetic construct
  REGION 1..118: note = Description of Artificial Sequence: Syntheticpolypeptide
QVQLVQSGAEVKKPGSSVKVSCKASGGTFSSYGFSWVRQAPGQGLEWMGGIIPIFGSANYAQKFQGRVTITADKSTSTVYMELISLRSEDTAVYYCARGGSSSPFAYWGQGTLVTVSS

SEQ ID NO: 3 - protein, 112 aa, synthetic construct
  REGION 1..112: note = Description of Artificial Sequence: Syntheticpolypeptide
DIVMTQTPLSSPVTLGQPASISCRSSQSLVHSDGNTYLSWLQQRPGQSPRLLIYKISNRFSGVPDRFSGSGAGTDFTLKISRVEAEDVGVYYCMQATQFPLTIGGGSKVEIK

SEQ ID NO: 4 - protein, 118 aa, synthetic construct
  REGION 1..118: note = Description of Artificial Sequence: Syntheticpolypeptide
QVQLVQSGAEVKKPGSSVKVSCKASGGTFSSYGFSWVRQAPGQGLEWMGGIIPIFGTANYAQKFQGRVTITADKSTSTVFMELISLRSEYTAVYYCARGGSSSPFAYWGQGTLVTVSS

SEQ ID NO: 5 - protein, 107 aa, synthetic construct
  REGION 1..107: note = Description of Artificial Sequence: Syntheticpolypeptide
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 6 - protein, 112 aa, synthetic construct
  REGION 1..112: note = Description of Artificial Sequence: Syntheticpolypeptide
QVQLQESGPGLVKPSETLSLTCTVSGGSISSYYWNWIRQPPGKGLEWIGYIYYSGSTNYNPSLKSRVTISVDTSKNQFSLKLRSVTAADTAVYYCARDIRTWGQGTLVTVSS

SEQ ID NO: 7 - protein, 112 aa, synthetic construct
  REGION 1..112: note = Description of Artificial Sequence: Syntheticpolypeptide
DVVMTQSPLSLPVTLGQPASISCRSSQSLVYSDENTYLNWFQQRPGQSLRRLIYQVSNRDSGVPDRFSGSGSGTDFTLKISRVEAEDVGVYFCMQGTYWPPTFGGGTKVEIK

SEQ ID NO: 8 - protein, 117 aa, synthetic construct
  REGION 1..117: note = Description of Artificial Sequence: Syntheticpolypeptide
EVQLVESGGGLVQPGGSLRLSCAASGFTFRNSWMSWVRQAPGKGLEWVANIKRDGSEKYYVDSVKGRFTISRDNAKNSLYLQMNSLRAEDTAVYYCARDQTGSFDYWGQGTLVTVSS

SEQ ID NO: 9 - protein, 106 aa, synthetic construct
  REGION 1..106: note = Description of Artificial Sequence: Syntheticpolypeptide
EIVMTQSPATLSMSLGERATLSCRASQSVSSSLAWYQQKPGQAPRLLIYGASTRATGIPARFGGSGSGTEFTLTISSLQSEDFAVYYCQQYINWPHFGGGTKVEIK

SEQ ID NO: 10 - protein, 115 aa, synthetic construct
  REGION 1..115: note = Description of Artificial Sequence: Syntheticpolypeptide
QVQLRGSGPGLVKPSETLSLTCTVSDDSISVYYWSWIRQPAGKGLEWIGRVYSSGNINYNPSLESRVTMSVDTSKSRFSLNLSSVTAADTAVYYCARGLDAFDIWGQGTMVTVSS

SEQ ID NO: 11 - protein, 107 aa, synthetic construct
  REGION 1..107: note = Description of Artificial Sequence: Syntheticpolypeptide
DIQMTQSPSSLSASVGDRVTITCQASQDISNYLNWYQQKPGKAPKILIYGASNLETGVPSRFSGSGSGTDFTFAISSLQPEDVATYYCQQYDNFPITFGQGTRLEIK

SEQ ID NO: 12 - protein, 115 aa, synthetic construct
  REGION 1..115: note = Description of Artificial Sequence: Syntheticpolypeptide
EVQLLESGGGLVQPGESLRLSCEVSGFTFTSYAMSWVRQVPGKGLEWVSIISGVAFTTYYADSVKGRFTISRDHSKNTLYLQMNGLRAEDTAVYYCVKVDGEVYWGQGTLVTVSS

SEQ ID NO: 13 - protein, 108 aa, synthetic construct
  REGION 1..108: note = Description of Artificial Sequence: Syntheticpolypeptide
EIVMTQSPDTLSVSPGERAILSCRASQSVSSNLAWYQQKPGQAPRLLIYSASTRASGIPARFSGSGSGTEFTLSISSLQSEDFAVYYCQQYDNWPPLTFGGGTKVEIK

SEQ ID NO: 14 - protein, 117 aa, synthetic construct
  REGION 1..117: note = Description of Artificial Sequence: Syntheticpolypeptide
QVQLVQSGAEVKKPGSSVKVSCEASGGTFITYAISWVRQAPGQGLEWMGGIIPFFGTANYAQKFQGRVTITADKSTSTASMELRSLRSEDTAMYYCAQWELFFFDFWGQGTPVTVSS

SEQ ID NO: 15 - protein, 108 aa, synthetic construct
  REGION 1..108: note = Description of Artificial Sequence: Syntheticpolypeptide
EIVLTQSPGTLSLSPGERATLSCRASQSVSSSYLAWYQQQPGQAPRLLIYGASSRATGIPDRFSGSGSGTDFTLTISRLEPEDFAIYYCQQYGSSPLTFGGGTKVEIK

SEQ ID NO: 16 - protein, 120 aa, synthetic construct
  REGION 1..120: note = Description of Artificial Sequence: Syntheticpolypeptide
AVQLVESGGGLVQPGGSLRLSCAASGFTFTYYSMNWVRQAPGKGLEWVSHISIRSSTIYFADSAKGRFTISRDNAKNSLYLQMNSLRDEDTAVYYCARGSGWYGDYFDYWGQGTLVTVSS

SEQ ID NO: 17 - protein, 107 aa, synthetic construct
  REGION 1..107: note = Description of Artificial Sequence: Syntheticpolypeptide
DIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 18 - protein, 123 aa, synthetic construct
  REGION 1..123: note = Description of Artificial Sequence: Syntheticpolypeptide
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSS

SEQ ID NO: 19 - protein, 107 aa, synthetic construct
  REGION 1..107: note = Description of Artificial Sequence: Syntheticpolypeptide
DIQMTQSPSSVSASVGDRVTITCRASQGISSWLAWYQQKPGKAPKVLIYAASSLQSGVPSRFSGSGSGTDFTLTISSLQPEDFATYYCQQAFSFPFTFGPGTKVDIK

SEQ ID NO: 20 - protein, 119 aa, synthetic construct
  REGION 1..119: note = Description of Artificial Sequence: Syntheticpolypeptide
EVQLVESGGGLVQPGGSLRLSCAVSGFTFSNHNIHWVRQAPGKGLEWISYISRSSSTIYYADSVKGRFTISRDNAKNSLYLQMNSLRDEDTAVYYCARDHAQWYGMDVWGQGTTVTVSS

SEQ ID NO: 21 - protein, 112 aa, synthetic construct
  REGION 1..112: note = Description of Artificial Sequence: Syntheticpolypeptide
DIVMTQSPDSLAVSLGERATINCKSSQSVLYSYNNKNYVAWYQQKPGQPPNLLIFWASTRESGVPDRFSGSGSGTDFTLTISSLQAEDVAVYYCQQYYSTLTFGGGTKVEIK

SEQ ID NO: 22 - protein, 124 aa, synthetic construct
  REGION 1..124: note = Description of Artificial Sequence: Syntheticpolypeptide
EVQLVESGGGLVQPGGSLRLSCVASGFTFSSYWMSWVRQAPGKGLEWVASIKQDGSEKYYVDSVKGRFTISRDNAKNSVYLQMNSLRAEDTGVYYCAREGVNWGWRLYWHFDLWGRGTLVTVSS

SEQ ID NO: 23 - protein, 108 aa, synthetic construct
  REGION 1..108: note = Description of Artificial Sequence: Syntheticpolypeptide
EIVLTQSPGTLSLSPGERVTLSCRASQSVSSSYLAWYQQKPGQAPRLLIYDASSRATGIPDRFSGSGSGTDFTLTISRLEPEDFAVYYCQQYGSSPLTFGGGTKVEIK

SEQ ID NO: 24 - protein, 120 aa, synthetic construct
  REGION 1..120: note = Description of Artificial Sequence: Syntheticpolypeptide
EVQVVESGGGLVQPGGSLRLSCAASGFTFSSYSMNWVRQAPGKGLEWVSHSSISRGNIYFADSVKGRFTISRDNAKNSLYLQMNSLRDEDTAVYYCARGSGWYGDYFDYWGQGTLVTVSS

SEQ ID NO: 25 - protein, 112 aa, synthetic construct
  REGION 1..112: note = Description of Artificial Sequence: Syntheticpolypeptide
ELQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVL

SEQ ID NO: 26 - protein, 120 aa, synthetic construct
  REGION 1..120: note = Description of Artificial Sequence: Syntheticpolypeptide
EVQLLESGGGLVQPGGSLRLSCAASGFAFSNYAMSWVRQAPGKGLEWVSAIRGGGGSTYYADSVKGRFTISRDNSKNTLYLQMNSLRAEDTAVYYCARDFISGTWYPDYWGQGTLVTVSS

SEQ ID NO: 27 - protein, 112 aa, synthetic construct
  REGION 1..112: note = Description of Artificial Sequence: Syntheticpolypeptide
ELQSVLTQPPSASGTPGQRVTISCSGSRSNIGSNYVYWYQQLPGTAPKLLIYRNNQRPSGVPDRFSGSKSGTSASLAISGLRSEDEADYYCASWDGSLSAVVFGTGTKLTVL

SEQ ID NO: 28 - protein, 122 aa, synthetic construct
  REGION 1..122: note = Description of Artificial Sequence: Syntheticpolypeptide
EVQLVQSGAEVKKPGESLKISCKGSGYRFTSYWIGWVRQMPGKGLEWMGSIYPDDSDTRYSPSFQGQVTISADKSISTAYLQWSSLKASDTAMYYCASSTVDYPGYSYFDYWGQGTLVTVSS

SEQ ID NO: 29 - protein, 112 aa, synthetic construct
  REGION 1..112: note = Description of Artificial Sequence: Syntheticpolypeptide
ELQSVLTQPPSASGTPGQRVTISCSGSSSNIGSNYVYWYQQLPGTAPKLLIYRNNQRPSGVPDRFSGSKSGTSASLAISGLRSEDEADYYCATWDDSLGSPVFGTGTKLTVL

SEQ ID NO: 30 - protein, 123 aa, synthetic construct
  REGION 1..123: note = Description of Artificial Sequence: Syntheticpolypeptide
EVQLVQSGAEVKKPGESLKISCKGSGYTFTNYWIAWVRQMPGKGLEWMGIIYPDGSDTRYSPSFQGQVTISADKSISTAYLQWSSLKASDTAMYYCARDITSWYYGEPAFDIWGQGTLVTVSS

SEQ ID NO: 31 - protein, 110 aa, synthetic construct
  REGION 1..110: note = Description of Artificial Sequence: Syntheticpolypeptide
ELDIQMTQSPSSLSASVGDRVTITCRASQSISRYLNWYQQKPGKAPKLLIYAASILQTGVPSRFSGSGSGTDFTLTISSLQPEDFATYYCQQSYSTTMWTFGQGTKVEIK

SEQ ID NO: 32 - protein, 122 aa, synthetic construct
  REGION 1..122: note = Description of Artificial Sequence: Syntheticpolypeptide
EVQLVQSGAEVKKPGESLKISCKGSGYSFTSSWIGWVRQMPGKGLEWMGIIYPGDSDTRYSPSFQGQVTISADKSISTAYLQWSSLKASDTAMYYCAKGLSQAMTGFGFDYWGQGTLVTVSS

SEQ ID NO: 33 - protein, 113 aa, synthetic construct
  REGION 1..113: note = Description of Artificial Sequence: Syntheticpolypeptide
ELQSVLTQPPSASGTPGQRVTISCSGSSSNIGSNYVNWYQQLPGTAPKLLIYGDYQRPSGVPDRFSGSKSGTSASLAISGLRSEDEADYYCATRDDSLSGSVVFGTGTKLTVL

SEQ ID NO: 34 - protein, 122 aa, synthetic construct
  REGION 1..122: note = Description of Artificial Sequence: Syntheticpolypeptide
EVQLVQSGAEVKKPGESLKISCKGSGYGFTSYWIGWVRQMPGKGLEWMGIIHPDDSDTKYSPSFQGQVTISADKSISTAYLQWSSLKASDTAMYYCASSYLRGLWGGYFDYWGQGTLVTVSS

SEQ ID NO: 35 - protein, 110 aa, synthetic construct
  REGION 1..110: note = Description of Artificial Sequence: Syntheticpolypeptide
ELDIQMTQSPSSLSASVGDRVTITCRASQSIYDYLHWYQQKPGKAPKLLIYDASNLQSGVPSRFSGSGSGTDFTLTISSLQPEDFATYYCQQSYTTPLFTFGQGTKVEIK

SEQ ID NO: 36 - protein, 126 aa, synthetic construct
  REGION 1..126: note = Description of Artificial Sequence: Syntheticpolypeptide
EVQLVQSGAEVKKPGESLKISCKGSGYTFPSSWIGWVRQMPGKGLEWMGIIYPDTSHTRYSPSFQGQVTISADKSISTAYLQWSSLKASDTAMYYCARASYFDRGTGYSSWWMDVWGQGTLVTVSS

SEQ ID NO: 37 - protein, 110 aa, synthetic construct
  REGION 1..110: note = Description of Artificial Sequence: Syntheticpolypeptide
ELDIQMTQSPSSLSASVGDRVTITCRASQYIGRYLNWYQQKRGKAPKLLIHGATSLASGVPSRFSGSGSGTDFTLTISSLQPEDFATYYCQQSYSTTSPTFGQGTKVEIK

SEQ ID NO: 38 - protein, 123 aa, synthetic construct
  REGION 1..123: note = Description of Artificial Sequence: Syntheticpolypeptide
EVQLLESGGGLVQPGGSLRLSCAASGFTFSQYSMSWVRQAPGKGLEWVSAISGGGVSTYYADSVKGRFTISRDNSKNTLYLQMNSLRAEDTAVYYCASDISDSGGSHWYFDYWGQGTLVTVSS

SEQ ID NO: 39 - protein, 112 aa, synthetic construct
  REGION 1..112: note = Description of Artificial Sequence: Syntheticpolypeptide
ELQSVLTQPPSASGTPGQRVTISCSGSTSNIGRNYVYWYQQLPGTAPKLLIYRTNQRPSGVPDRFSGSKSGTSASLAISGLRSEDEADYYCAAWDDSLSGRVFGTGTKLTVL

SEQ ID NO: 40 - protein, 121 aa, synthetic construct
  REGION 1..121: note = Description of Artificial Sequence: Syntheticpolypeptide
EVQLLESGGGLVQPGGSLRLSCAASGFTFSSYAMSWVRQAPGKGLEWVSTISGTGGTTYYADSVKGRFTISRDNSKNTLYLQMNSLRAEDTAVYYCAKVRAGIDPTASDVWGQGTLVTVSS

SEQ ID NO: 41 - protein, 112 aa, synthetic construct
  REGION 1..112: note = Description of Artificial Sequence: Syntheticpolypeptide
ELQSVLTQPPSASGTPGQRVTISCSGSSSNIGSNYVYWYQQLPGTAPKPLIYMNNQRPSGVPDRFSGSKSGTSASLAISGLRSEDEADYYCAAWDDSLSAVVFGTGTKLTVL

SEQ ID NO: 42 - protein, 124 aa, synthetic construct
  REGION 1..124: note = Description of Artificial Sequence: Syntheticpolypeptide
EVQLLESGGGLVQPGGSLRLSCAASGFTFNNFAMSWVRQAPGKGLEWVSGISGSGDNTYYADSVKGRFTISRDNSKNTLYLQMNSLRAEDTAVYYCAKDRDIGLGWYSYYLDVWGQGTLVTVSS

SEQ ID NO: 43 - protein, 113 aa, synthetic construct
  REGION 1..113: note = Description of Artificial Sequence: Syntheticpolypeptide
ELQSVLTQPPSASGTPGQRVTISCSGSNSNIGTNYVSWYQQLPGTAPKLLIYRSSRRPSGVPDRFSGSKSGTSASLAISGLRSEDEADYYCAAWDGSLSGHWVFGTGTKLTVL

SEQ ID NO: 44 - protein, 119 aa, synthetic construct
  REGION 1..119: note = Description of Artificial Sequence: Syntheticpolypeptide
QVQLVQSGAEVKKPGSSVKVSCKASGGTFSSYAISWVRQAPGQGLEWMGEIIPIFGTASYAQKFQGRVTITADESTSTAYMELSSLRSEDTAVYYCARAGWDDSWFDYWGQGTLVTVSS

SEQ ID NO: 45 - protein, 110 aa, synthetic construct
  REGION 1..110: note = Description of Artificial Sequence: Syntheticpolypeptide
ELDIQMTQSPSSLSASVGDRVTITCRASQSIDTYLNWYQQKPGKAPKLLIYSASSLHSGVPSRFSGSGSGTDFTLTISSLQPEDFATYYCQQSYSTTAWTFGQGTKVEIK

SEQ ID NO: 46 - protein, 122 aa, synthetic construct
  REGION 1..122: note = Description of Artificial Sequence: Syntheticpolypeptide
EVQLVQSGAEVKKPGESLKISCKGSGYSFASYWIGWVRQMPGKGLEWMGVIYPGTSETRYSPSFQGQVTISADKSISTAYLQWSSLKASDTAMYYCAKGLSASASGYSFQYWGQGTLVTVSS

SEQ ID NO: 47 - protein, 109 aa, synthetic construct
  REGION 1..109: note = Description of Artificial Sequence: Syntheticpolypeptide
ELDIQMTQSPSSLSASVGDRVTITCRASQSIGQSLNWYQQKPGKAPKLLIYGASSLQSGVPSRFSGSGSGTDFTLTISSLQPEDFATYYCQQSYSTPITFGQGTKVEIK

SEQ ID NO: 48 - protein, 124 aa, synthetic construct
  REGION 1..124: note = Description of Artificial Sequence: Syntheticpolypeptide
EVQLVQSGAEVKKPGESLKISCKGSGYSFTDYWIGWVRQMPGKGLEWMGMISPGGSTTIYRPSFQGQVTISADKSISTAYLQWSSLKASDTAMYYCAREMYTGGYGGSWYFDYWGQGTLVTVSS

SEQ ID NO: 49 - protein, 5 aa, synthetic construct
  REGION 1..5: note = Description of Artificial Sequence: Syntheticpeptide
SYGFS

SEQ ID NO: 50 - protein, 7 aa, synthetic construct
  REGION 1..7: note = Description of Artificial Sequence: Syntheticpeptide
GGTFSSY

SEQ ID NO: 51 - protein, 10 aa, synthetic construct
  REGION 1..10: note = Description of Artificial Sequence: Syntheticpeptide
GGTFSSYGFS

SEQ ID NO: 52 - protein, 17 aa, synthetic construct
  REGION 1..17: note = Description of Artificial Sequence: Syntheticpeptide
GIIPIFGSANYAQKFQG

SEQ ID NO: 53 - protein, 6 aa, synthetic construct
  REGION 1..6: note = Description of Artificial Sequence: Syntheticpeptide
IPIFGS

SEQ ID NO: 54 - protein, 9 aa, synthetic construct
  REGION 1..9: note = Description of Artificial Sequence: Syntheticpeptide
GGSSSPFAY

SEQ ID NO: 55 - protein, 5 aa, synthetic construct
  REGION 1..5: note = Description of Artificial Sequence: Syntheticpeptide
SYGFS

SEQ ID NO: 56 - protein, 7 aa, synthetic construct
  REGION 1..7: note = Description of Artificial Sequence: Syntheticpeptide
GGTFSSY

SEQ ID NO: 57 - protein, 10 aa, synthetic construct
  REGION 1..10: note = Description of Artificial Sequence: Syntheticpeptide
GGTFSSYGFS

SEQ ID NO: 58 - protein, 17 aa, synthetic construct
  REGION 1..17: note = Description of Artificial Sequence: Syntheticpeptide
GIIPIFGTANYAQKFQG

SEQ ID NO: 59 - protein, 6 aa, synthetic construct
  REGION 1..6: note = Description of Artificial Sequence: Syntheticpeptide
IPIFGT

SEQ ID NO: 60 - protein, 9 aa, synthetic construct
  REGION 1..9: note = Description of Artificial Sequence: Syntheticpeptide
GGSSSPFAY

SEQ ID NO: 61 - protein, 5 aa, synthetic construct
  REGION 1..5: note = Description of Artificial Sequence: Syntheticpeptide
SYYWN

SEQ ID NO: 62 - protein, 7 aa, synthetic construct
  REGION 1..7: note = Description of Artificial Sequence: Syntheticpeptide
GGSISSY

SEQ ID NO: 63 - protein, 10 aa, synthetic construct
  REGION 1..10: note = Description of Artificial Sequence: Syntheticpeptide
GGSISSYYWN

SEQ ID NO: 64 - protein, 16 aa, synthetic construct
  REGION 1..16: note = Description of Artificial Sequence: Syntheticpeptide
YIYYSGSTNYNPSLKS

SEQ ID NO: 65 - protein, 5 aa, synthetic construct
  REGION 1..5: note = Description of Artificial Sequence: Syntheticpeptide
YYSGS

SEQ ID NO: 66 - protein, 5 aa, synthetic construct
  REGION 1..5: note = Description of Artificial Sequence: Syntheticpeptide
DIRTW

SEQ ID NO: 67 - protein, 5 aa, synthetic construct
  REGION 1..5: note = Description of Artificial Sequence: Syntheticpeptide
NSWMS

SEQ ID NO: 68 - protein, 7 aa, synthetic construct
  REGION 1..7: note = Description of Artificial Sequence: Syntheticpeptide
GFTFRNS

SEQ ID NO: 69 - protein, 10 aa, synthetic construct
  REGION 1..10: note = Description of Artificial Sequence: Syntheticpeptide
GFTFRNSWMS

SEQ ID NO: 70 - protein, 17 aa, synthetic construct
  REGION 1..17: note = Description of Artificial Sequence: Syntheticpeptide
NIKRDGSEKYYVDSVKG

SEQ ID NO: 71 - protein, 6 aa, synthetic construct
  REGION 1..6: note = Description of Artificial Sequence: Syntheticpeptide
KRDGSE

SEQ ID NO: 72 - protein, 8 aa, synthetic construct
  REGION 1..8: note = Description of Artificial Sequence: Syntheticpeptide
DQTGSFDY

SEQ ID NO: 73 - protein, 5 aa, synthetic construct
  REGION 1..5: note = Description of Artificial Sequence: Syntheticpeptide
VYYWS

SEQ ID NO: 74 - protein, 7 aa, synthetic construct
  REGION 1..7: note = Description of Artificial Sequence: Syntheticpeptide
DDSISVY

SEQ ID NO: 75 - protein, 10 aa, synthetic construct
  REGION 1..10: note = Description of Artificial Sequence: Syntheticpeptide
DDSISVYYWS

SEQ ID NO: 76 - protein, 15 aa, synthetic construct
  REGION 1..15: note = Description of Artificial Sequence: Syntheticpeptide
VYSSGNINYNPSLES

SEQ ID NO: 77 - protein, 5 aa, synthetic construct
  REGION 1..5: note = Description of Artificial Sequence: Syntheticpeptide
YSSGN

SEQ ID NO: 78 - protein, 7 aa, synthetic construct
  REGION 1..7: note = Description of Artificial Sequence: Syntheticpeptide
GLDAFDI

SEQ ID NO: 79 - protein, 5 aa, synthetic construct
  REGION 1..5: note = Description of Artificial Sequence: Syntheticpeptide
SYAMS

SEQ ID NO: 80 - protein, 7 aa, synthetic construct
  REGION 1..7: note = Description of Artificial Sequence: Syntheticpeptide
GFTFTSY

SEQ ID NO: 81 - protein, 10 aa, synthetic construct
  REGION 1..10: note = Description of Artificial Sequence: Syntheticpeptide
GFTFTSYAMS

SEQ ID NO: 82 - protein, 16 aa, synthetic construct
  REGION 1..16: note = Description of Artificial Sequence: Syntheticpeptide
RVYSSGNINYNPSLES

SEQ ID NO: 83 - protein, 5 aa, synthetic construct
  REGION 1..5: note = Description of Artificial Sequence: Syntheticpeptide
YSSGN

SEQ ID NO: 84 - protein, 7 aa, synthetic construct
  REGION 1..7: note = Description of Artificial Sequence: Syntheticpeptide
GLDAFDI

SEQ ID NO: 85 - protein, 5 aa, synthetic construct
  REGION 1..5: note = Description of Artificial Sequence: Syntheticpeptide
TYAIS

SEQ ID NO: 86 - protein, 7 aa, synthetic construct
  REGION 1..7: note = Description of Artificial Sequence: Syntheticpeptide
GGTFITY

SEQ ID NO: 87 - protein, 10 aa, synthetic construct
  REGION 1..10: note = Description of Artificial Sequence: Syntheticpeptide
GGTFITYAIS

SEQ ID NO: 88 - protein, 17 aa, synthetic construct
  REGION 1..17: note = Description of Artificial Sequence: Syntheticpeptide
GIIPFFGTANYAQKFQG

SEQ ID NO: 89 - protein, 6 aa, synthetic construct
  REGION 1..6: note = Description of Artificial Sequence: Syntheticpeptide
IPFFGT

SEQ ID NO: 90 - protein, 8 aa, synthetic construct
  REGION 1..8: note = Description of Artificial Sequence: Syntheticpeptide
WELFFFDF

SEQ ID NO: 91 - protein, 5 aa, synthetic construct
  REGION 1..5: note = Description of Artificial Sequence: Syntheticpeptide
YYSMN

SEQ ID NO: 92 - protein, 7 aa, synthetic construct
  REGION 1..7: note = Description of Artificial Sequence: Syntheticpeptide
GFTFTYY

SEQ ID NO: 93 - protein, 10 aa, synthetic construct
  REGION 1..10: note = Description of Artificial Sequence: Syntheticpeptide
GFTFTYYSMN

SEQ ID NO: 94 - protein, 17 aa, synthetic construct
  REGION 1..17: note = Description of Artificial Sequence: Syntheticpeptide
HISIRSSTIYFADSAKG

SEQ ID NO: 95 - protein, 6 aa, synthetic construct
  REGION 1..6: note = Description of Artificial Sequence: Syntheticpeptide
SIRSST

SEQ ID NO: 96 - protein, 11 aa, synthetic construct
  REGION 1..11: note = Description of Artificial Sequence: Syntheticpeptide
GSGWYGDYFDY

SEQ ID NO: 97 - protein, 7 aa, synthetic construct
  REGION 1..7: note = Description of Artificial Sequence: Syntheticpeptide
NARMGVT

SEQ ID NO: 98 - protein, 9 aa, synthetic construct
  REGION 1..9: note = Description of Artificial Sequence: Syntheticpeptide
GFSLSNARM

SEQ ID NO: 99 - protein, 12 aa, synthetic construct
  REGION 1..12: note = Description of Artificial Sequence: Syntheticpeptide
GFSLSNARMGVT

SEQ ID NO: 100 - protein, 16 aa, synthetic construct
  REGION 1..16: note = Description of Artificial Sequence: Syntheticpeptide
HIFSNDEKSYSTSLKS

SEQ ID NO: 101 - protein, 5 aa, synthetic construct
  REGION 1..5: note = Description of Artificial Sequence: Syntheticpeptide
FSNDE

SEQ ID NO: 102 - protein, 13 aa, synthetic construct
  REGION 1..13: note = Description of Artificial Sequence: Syntheticpeptide
IRDYYDISSYYDY

SEQ ID NO: 103 - protein, 5 aa, synthetic construct
  REGION 1..5: note = Description of Artificial Sequence: Syntheticpeptide
NHNIH

SEQ ID NO: 104 - protein, 7 aa, synthetic construct
  REGION 1..7: note = Description of Artificial Sequence: Syntheticpeptide
GFTFSNH

SEQ ID NO: 105 - protein, 10 aa, synthetic construct
  REGION 1..10: note = Description of Artificial Sequence: Syntheticpeptide
GFTFSNHNIH

SEQ ID NO: 106 - protein, 17 aa, synthetic construct
  REGION 1..17: note = Description of Artificial Sequence: Syntheticpeptide
YISRSSSTIYYADSVKG

SEQ ID NO: 107 - protein, 6 aa, synthetic construct
  REGION 1..6: note = Description of Artificial Sequence: Syntheticpeptide
SRSSST

SEQ ID NO: 108 - protein, 10 aa, synthetic construct
  REGION 1..10: note = Description of Artificial Sequence: Syntheticpeptide
DHAQWYGMDV

SEQ ID NO: 109 - protein, 5 aa, synthetic construct
  REGION 1..5: note = Description of Artificial Sequence: Syntheticpeptide
SYWMS

SEQ ID NO: 110 - protein, 7 aa, synthetic construct
  REGION 1..7: note = Description of Artificial Sequence: Syntheticpeptide
GFTFSSY

SEQ ID NO: 111 - protein, 10 aa, synthetic construct
  REGION 1..10: note = Description of Artificial Sequence: Syntheticpeptide
GFTFSSYWMS

SEQ ID NO: 112 - protein, 17 aa, synthetic construct
  REGION 1..17: note = Description of Artificial Sequence: Syntheticpeptide
SIKQDGSEKYYVDSVKG

SEQ ID NO: 113 - protein, 6 aa, synthetic construct
  REGION 1..6: note = Description of Artificial Sequence: Syntheticpeptide
KQDGSE

SEQ ID NO: 114 - protein, 15 aa, synthetic construct
  REGION 1..15: note = Description of Artificial Sequence: Syntheticpeptide
EGVNWGWRLYWHFDL

SEQ ID NO: 115 - protein, 5 aa, synthetic construct
  REGION 1..5: note = Description of Artificial Sequence: Syntheticpeptide
SYSMN

SEQ ID NO: 116 - protein, 7 aa, synthetic construct
  REGION 1..7: note = Description of Artificial Sequence: Syntheticpeptide
GFTFSSY

SEQ ID NO: 117 - protein, 10 aa, synthetic construct
  REGION 1..10: note = Description of Artificial Sequence: Syntheticpeptide
GFTFSSYSMN

SEQ ID NO: 118 - protein, 17 aa, synthetic construct
  REGION 1..17: note = Description of Artificial Sequence: Syntheticpeptide
HSSISRGNIYFADSVKG

SEQ ID NO: 119 - protein, 6 aa, synthetic construct
  REGION 1..6: note = Description of Artificial Sequence: Syntheticpeptide
SISRGN

SEQ ID NO: 120 - protein, 11 aa, synthetic construct
  REGION 1..11: note = Description of Artificial Sequence: Syntheticpeptide
GSGWYGDYFDY

SEQ ID NO: 121 - protein, 5 aa, synthetic construct
  REGION 1..5: note = Description of Artificial Sequence: Syntheticpeptide
NYAMS

SEQ ID NO: 122 - protein, 7 aa, synthetic construct
  REGION 1..7: note = Description of Artificial Sequence: Syntheticpeptide
GFAFSNY

SEQ ID NO: 123 - protein, 10 aa, synthetic construct
  REGION 1..10: note = Description of Artificial Sequence: Syntheticpeptide
GFAFSNYAMS

SEQ ID NO: 124 - protein, 17 aa, synthetic construct
  REGION 1..17: note = Description of Artificial Sequence: Syntheticpeptide
AIRGGGGSTYYADSVKG

SEQ ID NO: 125 - protein, 6 aa, synthetic construct
  REGION 1..6: note = Description of Artificial Sequence: Syntheticpeptide
RGGGGS

SEQ ID NO: 126 - protein, 11 aa, synthetic construct
  REGION 1..11: note = Description of Artificial Sequence: Syntheticpeptide
DFISGTWYPDY

SEQ ID NO: 127 - protein, 5 aa, synthetic construct
  REGION 1..5: note = Description of Artificial Sequence: Syntheticpeptide
SYWIG

SEQ ID NO: 128 - protein, 7 aa, synthetic construct
  REGION 1..7: note = Description of Artificial Sequence: Syntheticpeptide
GYRFTSY

SEQ ID NO: 129 - protein, 10 aa, synthetic construct
  REGION 1..10: note = Description of Artificial Sequence: Syntheticpeptide
GYRFTSYWIG

SEQ ID NO: 130 - protein, 17 aa, synthetic construct
  REGION 1..17: note = Description of Artificial Sequence: Syntheticpeptide
SIYPDDSDTRYSPSFQG

SEQ ID NO: 131 - protein, 6 aa, synthetic construct
  REGION 1..6: note = Description of Artificial Sequence: Syntheticpeptide
YPDDSD

SEQ ID NO: 132 - protein, 13 aa, synthetic construct
  REGION 1..13: note = Description of Artificial Sequence: Syntheticpeptide
STVDYPGYSYFDY

SEQ ID NO: 133 - protein, 5 aa, synthetic construct
  REGION 1..5: note = Description of Artificial Sequence: Syntheticpeptide
NYWIA

SEQ ID NO: 134 - protein, 7 aa, synthetic construct
  REGION 1..7: note = Description of Artificial Sequence: Syntheticpeptide
GYTFTNY

SEQ ID NO: 135 - protein, 10 aa, synthetic construct
  REGION 1..10: note = Description of Artificial Sequence: Syntheticpeptide
GYTFTNYWIA

SEQ ID NO: 136 - protein, 17 aa, synthetic construct
  REGION 1..17: note = Description of Artificial Sequence: Syntheticpeptide
IIYPDGSDTRYSPSFQG

SEQ ID NO: 137 - protein, 6 aa, synthetic construct
  REGION 1..6: note = Description of Artificial Sequence: Syntheticpeptide
YPDGSD

SEQ ID NO: 138 - protein, 14 aa, synthetic construct
  REGION 1..14: note = Description of Artificial Sequence: Syntheticpeptide
DITSWYYGEPAFDI

SEQ ID NO: 139 - protein, 5 aa, synthetic construct
  REGION 1..5: note = Description of Artificial Sequence: Syntheticpeptide
SSWIG

SEQ ID NO: 140 - protein, 7 aa, synthetic construct
  REGION 1..7: note = Description of Artificial Sequence: Syntheticpeptide
GYSFTSS

SEQ ID NO: 141 - protein, 10 aa, synthetic construct
  REGION 1..10: note = Description of Artificial Sequence: Syntheticpeptide
GYSFTSSWIG

SEQ ID NO: 142 - protein, 17 aa, synthetic construct
  REGION 1..17: note = Description of Artificial Sequence: Syntheticpeptide
IIYPGDSDTRYSPSFQG

SEQ ID NO: 143 - protein, 6 aa, synthetic construct
  REGION 1..6: note = Description of Artificial Sequence: Syntheticpeptide
YPGDSD

SEQ ID NO: 144 - protein, 13 aa, synthetic construct
  REGION 1..13: note = Description of Artificial Sequence: Syntheticpeptide
GLSQAMTGFGFDY

SEQ ID NO: 145 - protein, 5 aa, synthetic construct
  REGION 1..5: note = Description of Artificial Sequence: Syntheticpeptide
SYWIG

SEQ ID NO: 146 - protein, 7 aa, synthetic construct
  REGION 1..7: note = Description of Artificial Sequence: Syntheticpeptide
GYGFTSY

SEQ ID NO: 147 - protein, 10 aa, synthetic construct
  REGION 1..10: note = Description of Artificial Sequence: Syntheticpeptide
GYGFTSYWIG

SEQ ID NO: 148 - protein, 17 aa, synthetic construct
  REGION 1..17: note = Description of Artificial Sequence: Syntheticpeptide
IIHPDDSDTKYSPSFQG

SEQ ID NO: 149 - protein, 6 aa, synthetic construct
  REGION 1..6: note = Description of Artificial Sequence: Syntheticpeptide
HPDDSD

SEQ ID NO: 150 - protein, 13 aa, synthetic construct
  REGION 1..13: note = Description of Artificial Sequence: Syntheticpeptide
SYLRGLWGGYFDY

SEQ ID NO: 151 - protein, 5 aa, synthetic construct
  REGION 1..5: note = Description of Artificial Sequence: Syntheticpeptide
SSWIG

SEQ ID NO: 152 - protein, 7 aa, synthetic construct
  REGION 1..7: note = Description of Artificial Sequence: Syntheticpeptide
GYTFPSS

SEQ ID NO: 153 - protein, 10 aa, synthetic construct
  REGION 1..10: note = Description of Artificial Sequence: Syntheticpeptide
GYTFPSSWIG

SEQ ID NO: 154 - protein, 16 aa, synthetic construct
  REGION 1..16: note = Description of Artificial Sequence: Syntheticpeptide
IIYPDTSHTRYSPSFQ

SEQ ID NO: 155 - protein, 6 aa, synthetic construct
  REGION 1..6: note = Description of Artificial Sequence: Syntheticpeptide
YPDTSH

SEQ ID NO: 156 - protein, 17 aa, synthetic construct
  REGION 1..17: note = Description of Artificial Sequence: Syntheticpeptide
ASYFDRGTGYSSWWMDV

SEQ ID NO: 157 - protein, 5 aa, synthetic construct
  REGION 1..5: note = Description of Artificial Sequence: Syntheticpeptide
QYSMS

SEQ ID NO: 158 - protein, 7 aa, synthetic construct
  REGION 1..7: note = Description of Artificial Sequence: Syntheticpeptide
GFTFSQY

SEQ ID NO: 159 - protein, 10 aa, synthetic construct
  REGION 1..10: note = Description of Artificial Sequence: Syntheticpeptide
GFTFSQYSMS

SEQ ID NO: 160 - protein, 17 aa, synthetic construct
  REGION 1..17: note = Description of Artificial Sequence: Syntheticpeptide
AISGGGVSTYYADSVKG

SEQ ID NO: 161 - protein, 6 aa, synthetic construct
  REGION 1..6: note = Description of Artificial Sequence: Syntheticpeptide
SGGGVS

SEQ ID NO: 162 - protein, 14 aa, synthetic construct
  REGION 1..14: note = Description of Artificial Sequence: Syntheticpeptide
DISDSGGSHWYFDY

SEQ ID NO: 163 - protein, 5 aa, synthetic construct
  REGION 1..5: note = Description of Artificial Sequence: Syntheticpeptide
SYAMS

SEQ ID NO: 164 - protein, 7 aa, synthetic construct
  REGION 1..7: note = Description of Artificial Sequence: Syntheticpeptide
GFTFSSY

SEQ ID NO: 165 - protein, 10 aa, synthetic construct
  REGION 1..10: note = Description of Artificial Sequence: Syntheticpeptide
GFTFSSYAMS

SEQ ID NO: 166 - protein, 17 aa, synthetic construct
  REGION 1..17: note = Description of Artificial Sequence: Syntheticpeptide
TISGTGGTTYYADSVKG

SEQ ID NO: 167 - protein, 6 aa, synthetic construct
  REGION 1..6: note = Description of Artificial Sequence: Syntheticpeptide
SGTGGT

SEQ ID NO: 168 - protein, 12 aa, synthetic construct
  REGION 1..12: note = Description of Artificial Sequence: Syntheticpeptide
VRAGIDPTASDV

SEQ ID NO: 169 - protein, 5 aa, synthetic construct
  REGION 1..5: note = Description of Artificial Sequence: Syntheticpeptide
NFAMS

SEQ ID NO: 170 - protein, 7 aa, synthetic construct
  REGION 1..7: note = Description of Artificial Sequence: Syntheticpeptide
GFTFNNF

SEQ ID NO: 171 - protein, 10 aa, synthetic construct
  REGION 1..10: note = Description of Artificial Sequence: Syntheticpeptide
GFTFNNFAMS

SEQ ID NO: 172 - protein, 17 aa, synthetic construct
  REGION 1..17: note = Description of Artificial Sequence: Syntheticpeptide
GISGSGDNTYYADSVKG

SEQ ID NO: 173 - protein, 6 aa, synthetic construct
  REGION 1..6: note = Description of Artificial Sequence: Syntheticpeptide
SGSGDN

SEQ ID NO: 174 - protein, 15 aa, synthetic construct
  REGION 1..15: note = Description of Artificial Sequence: Syntheticpeptide
DRDIGLGWYSYYLDV

SEQ ID NO: 175 - protein, 5 aa, synthetic construct
  REGION 1..5: note = Description of Artificial Sequence: Syntheticpeptide
SYAIS

SEQ ID NO: 176 - protein, 7 aa, synthetic construct
  REGION 1..7: note = Description of Artificial Sequence: Syntheticpeptide
GGTFSSY

SEQ ID NO: 177 - protein, 10 aa, synthetic construct
  REGION 1..10: note = Description of Artificial Sequence: Syntheticpeptide
GGTFSSYAIS

SEQ ID NO: 178 - protein, 17 aa, synthetic construct
  REGION 1..17: note = Description of Artificial Sequence: Syntheticpeptide
EIIPIFGTASYAQKFQG

SEQ ID NO: 179 - protein, 6 aa, synthetic construct
  REGION 1..6: note = Description of Artificial Sequence: Syntheticpeptide
IPIFGT

SEQ ID NO: 180 - protein, 10 aa, synthetic construct
  REGION 1..10: note = Description of Artificial Sequence: Syntheticpeptide
AGWDDSWFDY

SEQ ID NO: 181 - protein, 5 aa, synthetic construct
  REGION 1..5: note = Description of Artificial Sequence: Syntheticpeptide
SYWIG

SEQ ID NO: 182 - protein, 7 aa, synthetic construct
  REGION 1..7: note = Description of Artificial Sequence: Syntheticpeptide
GYSFASY

SEQ ID NO: 183 - protein, 10 aa, synthetic construct
  REGION 1..10: note = Description of Artificial Sequence: Syntheticpeptide
GYSFASYWIG

SEQ ID NO: 184 - protein, 17 aa, synthetic construct
  REGION 1..17: note = Description of Artificial Sequence: Syntheticpeptide
VIYPGTSETRYSPSFQG

SEQ ID NO: 185 - protein, 6 aa, synthetic construct
  REGION 1..6: note = Description of Artificial Sequence: Syntheticpeptide
YPGTSE

SEQ ID NO: 186 - protein, 13 aa, synthetic construct
  REGION 1..13: note = Description of Artificial Sequence: Syntheticpeptide
GLSASASGYSFQY

SEQ ID NO: 187 - protein, 5 aa, synthetic construct
  REGION 1..5: note = Description of Artificial Sequence: Syntheticpeptide
DYWIG

SEQ ID NO: 188 - protein, 7 aa, synthetic construct
  REGION 1..7: note = Description of Artificial Sequence: Syntheticpeptide
GYSFTDY

SEQ ID NO: 189 - protein, 10 aa, synthetic construct
  REGION 1..10: note = Description of Artificial Sequence: Syntheticpeptide
GYSFTDYWIG

SEQ ID NO: 190 - protein, 17 aa, synthetic construct
  REGION 1..17: note = Description of Artificial Sequence: Syntheticpeptide
MISPGGSTTIYRPSFQG

SEQ ID NO: 191 - protein, 6 aa, synthetic construct
  REGION 1..6: note = Description of Artificial Sequence: Syntheticpeptide
SPGGST

SEQ ID NO: 192 - protein, 14 aa, synthetic construct
  REGION 1..14: note = Description of Artificial Sequence: Syntheticpeptide
MYTGGYGGSWYFDY

SEQ ID NO: 193 - protein, 16 aa, synthetic construct
  REGION 1..16: note = Description of Artificial Sequence: Syntheticpeptide
RSSQSLVHSDGNTYLS

SEQ ID NO: 194 - protein, 7 aa, synthetic construct
  REGION 1..7: note = Description of Artificial Sequence: Syntheticpeptide
KISNRFS

SEQ ID NO: 195 - protein, 9 aa, synthetic construct
  REGION 1..9: note = Description of Artificial Sequence: Syntheticpeptide
MQATQFPLT

SEQ ID NO: 196 - protein, 16 aa, synthetic construct
  REGION 1..16: note = Description of Artificial Sequence: Syntheticpeptide
RSSQSLVHSDGNTYLS

SEQ ID NO: 197 - protein, 7 aa, synthetic construct
  REGION 1..7: note = Description of Artificial Sequence: Syntheticpeptide
KISNRFS

SEQ ID NO: 198 - protein, 9 aa, synthetic construct
  REGION 1..9: note = Description of Artificial Sequence: Syntheticpeptide
MQATQFPLT

SEQ ID NO: 199 - protein, 11 aa, synthetic construct
  REGION 1..11: note = Description of Artificial Sequence: Syntheticpeptide
RASQGISNYLA

SEQ ID NO: 200 - protein, 7 aa, synthetic construct
  REGION 1..7: note = Description of Artificial Sequence: Syntheticpeptide
AASSLQS

SEQ ID NO: 201 - protein, 9 aa, synthetic construct
  REGION 1..9: note = Description of Artificial Sequence: Syntheticpeptide
QQYNSYPLT

SEQ ID NO: 202 - protein, 16 aa, synthetic construct
  REGION 1..16: note = Description of Artificial Sequence: Syntheticpeptide
RSSQSLVYSDENTYLN

SEQ ID NO: 203 - protein, 7 aa, synthetic construct
  REGION 1..7: note = Description of Artificial Sequence: Syntheticpeptide
QVSNRDS

SEQ ID NO: 204 - protein, 9 aa, synthetic construct
  REGION 1..9: note = Description of Artificial Sequence: Syntheticpeptide
MQGTYWPPT

SEQ ID NO: 205 - protein, 11 aa, synthetic construct
  REGION 1..11: note = Description of Artificial Sequence: Syntheticpeptide
RASQSVSSSLA

SEQ ID NO: 206 - protein, 7 aa, synthetic construct
  REGION 1..7: note = Description of Artificial Sequence: Syntheticpeptide
GASTRAT

SEQ ID NO: 207 - protein, 8 aa, synthetic construct
  REGION 1..8: note = Description of Artificial Sequence: Syntheticpeptide
QQYINWPH

SEQ ID NO: 208 - protein, 11 aa, synthetic construct
  REGION 1..11: note = Description of Artificial Sequence: Syntheticpeptide
QASQDISNYLN

SEQ ID NO: 209 - protein, 7 aa, synthetic construct
  REGION 1..7: note = Description of Artificial Sequence: Syntheticpeptide
GASNLET

SEQ ID NO: 210 - protein, 9 aa, synthetic construct
  REGION 1..9: note = Description of Artificial Sequence: Syntheticpeptide
QQYDNFPIT

SEQ ID NO: 211 - protein, 11 aa, synthetic construct
  REGION 1..11: note = Description of Artificial Sequence: Syntheticpeptide
RASQSVSSNLA

SEQ ID NO: 212 - protein, 7 aa, synthetic construct
  REGION 1..7: note = Description of Artificial Sequence: Syntheticpeptide
SASTRAS

SEQ ID NO: 213 - protein, 10 aa, synthetic construct
  REGION 1..10: note = Description of Artificial Sequence: Syntheticpeptide
QQYDNWPPLT

SEQ ID NO: 214 - protein, 12 aa, synthetic construct
  REGION 1..12: note = Description of Artificial Sequence: Syntheticpeptide
RASQSVSSSYLA

SEQ ID NO: 215 - protein, 7 aa, synthetic construct
  REGION 1..7: note = Description of Artificial Sequence: Syntheticpeptide
GASSRAT

SEQ ID NO: 216 - protein, 9 aa, synthetic construct
  REGION 1..9: note = Description of Artificial Sequence: Syntheticpeptide
QQYGSSPLT

SEQ ID NO: 217 - protein, 11 aa, synthetic construct
  REGION 1..11: note = Description of Artificial Sequence: Syntheticpeptide
RASQDISNYLA

SEQ ID NO: 218 - protein, 7 aa, synthetic construct
  REGION 1..7: note = Description of Artificial Sequence: Syntheticpeptide
AASSLQS

SEQ ID NO: 219 - protein, 9 aa, synthetic construct
  REGION 1..9: note = Description of Artificial Sequence: Syntheticpeptide
LQLNSFPFT

SEQ ID NO: 220 - protein, 11 aa, synthetic construct
  REGION 1..11: note = Description of Artificial Sequence: Syntheticpeptide
RASQGISSWLA

SEQ ID NO: 221 - protein, 7 aa, synthetic construct
  REGION 1..7: note = Description of Artificial Sequence: Syntheticpeptide
AASSLQS

SEQ ID NO: 222 - protein, 9 aa, synthetic construct
  REGION 1..9: note = Description of Artificial Sequence: Syntheticpeptide
QQAFSFPFT

SEQ ID NO: 223 - protein, 17 aa, synthetic construct
  REGION 1..17: note = Description of Artificial Sequence: Syntheticpeptide
KSSQSVLYSYNNKNYVA

SEQ ID NO: 224 - protein, 7 aa, synthetic construct
  REGION 1..7: note = Description of Artificial Sequence: Syntheticpeptide
WASTRES

SEQ ID NO: 225 - protein, 8 aa, synthetic construct
  REGION 1..8: note = Description of Artificial Sequence: Syntheticpeptide
QQYYSTLT

SEQ ID NO: 226 - protein, 12 aa, synthetic construct
  REGION 1..12: note = Description of Artificial Sequence: Syntheticpeptide
RASQSVSSSYLA

SEQ ID NO: 227 - protein, 7 aa, synthetic construct
  REGION 1..7: note = Description of Artificial Sequence: Syntheticpeptide
DASSRAT

SEQ ID NO: 228 - protein, 9 aa, synthetic construct
  REGION 1..9: note = Description of Artificial Sequence: Syntheticpeptide
QQYGSSPLT

SEQ ID NO: 229 - protein, 13 aa, synthetic construct
  REGION 1..13: note = Description of Artificial Sequence: Syntheticpeptide
SGSSSNIGSNYVY

SEQ ID NO: 230 - protein, 7 aa, synthetic construct
  REGION 1..7: note = Description of Artificial Sequence: Syntheticpeptide
RNNQRPS

SEQ ID NO: 231 - protein, 11 aa, synthetic construct
  REGION 1..11: note = Description of Artificial Sequence: Syntheticpeptide
AAWDDSLSGVV

SEQ ID NO: 232 - protein, 13 aa, synthetic construct
  REGION 1..13: note = Description of Artificial Sequence: Syntheticpeptide
SGSRSNIGSNYVY

SEQ ID NO: 233 - protein, 7 aa, synthetic construct
  REGION 1..7: note = Description of Artificial Sequence: Syntheticpeptide
RNNQRPS

SEQ ID NO: 234 - protein, 11 aa, synthetic construct
  REGION 1..11: note = Description of Artificial Sequence: Syntheticpeptide
ASWDGSLSAVV

SEQ ID NO: 235 - protein, 13 aa, synthetic construct
  REGION 1..13: note = Description of Artificial Sequence: Syntheticpeptide
SGSSSNIGSNYVY

SEQ ID NO: 236 - protein, 7 aa, synthetic construct
  REGION 1..7: note = Description of Artificial Sequence: Syntheticpeptide
RNNQRPS

SEQ ID NO: 237 - protein, 11 aa, synthetic construct
  REGION 1..11: note = Description of Artificial Sequence: Syntheticpeptide
ATWDDSLGSPV

SEQ ID NO: 238 - protein, 11 aa, synthetic construct
  REGION 1..11: note = Description of Artificial Sequence: Syntheticpeptide
RASQSISRYLN

SEQ ID NO: 239 - protein, 7 aa, synthetic construct
  REGION 1..7: note = Description of Artificial Sequence: Syntheticpeptide
AASILQT

SEQ ID NO: 240 - protein, 10 aa, synthetic construct
  REGION 1..10: note = Description of Artificial Sequence: Syntheticpeptide
QQSYSTTMWT

SEQ ID NO: 241 - protein, 13 aa, synthetic construct
  REGION 1..13: note = Description of Artificial Sequence: Syntheticpeptide
SGSSSNIGSNYVN

SEQ ID NO: 242 - protein, 7 aa, synthetic construct
  REGION 1..7: note = Description of Artificial Sequence: Syntheticpeptide
GDYQRPS

SEQ ID NO: 243 - protein, 12 aa, synthetic construct
  REGION 1..12: note = Description of Artificial Sequence: Syntheticpeptide
ATRDDSLSGSVV

SEQ ID NO: 244 - protein, 11 aa, synthetic construct
  REGION 1..11: note = Description of Artificial Sequence: Syntheticpeptide
RASQSIYDYLH

SEQ ID NO: 245 - protein, 7 aa, synthetic construct
  REGION 1..7: note = Description of Artificial Sequence: Syntheticpeptide
DASNLQS

SEQ ID NO: 246 - protein, 10 aa, synthetic construct
  REGION 1..10: note = Description of Artificial Sequence: Syntheticpeptide
QQSYTTPLFT

SEQ ID NO: 247 - protein, 11 aa, synthetic construct
  REGION 1..11: note = Description of Artificial Sequence: Syntheticpeptide
RASQYIGRYLN

SEQ ID NO: 248 - protein, 7 aa, synthetic construct
  REGION 1..7: note = Description of Artificial Sequence: Syntheticpeptide
GATSLAS

SEQ ID NO: 249 - protein, 10 aa, synthetic construct
  REGION 1..10: note = Description of Artificial Sequence: Syntheticpeptide
QQSYSTTSPT

SEQ ID NO: 250 - protein, 13 aa, synthetic construct
  REGION 1..13: note = Description of Artificial Sequence: Syntheticpeptide
SGSTSNIGRNYVY

SEQ ID NO: 251 - protein, 7 aa, synthetic construct
  REGION 1..7: note = Description of Artificial Sequence: Syntheticpeptide
RTNQRPS

SEQ ID NO: 252 - protein, 11 aa, synthetic construct
  REGION 1..11: note = Description of Artificial Sequence: Syntheticpeptide
AAWDDSLSGRV

SEQ ID NO: 253 - protein, 13 aa, synthetic construct
  REGION 1..13: note = Description of Artificial Sequence: Syntheticpeptide
SGSSSNIGSNYVY

SEQ ID NO: 254 - protein, 7 aa, synthetic construct
  REGION 1..7: note = Description of Artificial Sequence: Syntheticpeptide
MNNQRPS

SEQ ID NO: 255 - protein, 11 aa, synthetic construct
  REGION 1..11: note = Description of Artificial Sequence: Syntheticpeptide
AAWDDSLSAVV

SEQ ID NO: 256 - protein, 13 aa, synthetic construct
  REGION 1..13: note = Description of Artificial Sequence: Syntheticpeptide
SGSNSNIGTNYVS

SEQ ID NO: 257 - protein, 7 aa, synthetic construct
  REGION 1..7: note = Description of Artificial Sequence: Syntheticpeptide
RSSRRPS

SEQ ID NO: 258 - protein, 12 aa, synthetic construct
  REGION 1..12: note = Description of Artificial Sequence: Syntheticpeptide
AAWDGSLSGHWV

SEQ ID NO: 259 - protein, 11 aa, synthetic construct
  REGION 1..11: note = Description of Artificial Sequence: Syntheticpeptide
RASQSIDTYLN

SEQ ID NO: 260 - protein, 7 aa, synthetic construct
  REGION 1..7: note = Description of Artificial Sequence: Syntheticpeptide
SASSLHS

SEQ ID NO: 261 - protein, 10 aa, synthetic construct
  REGION 1..10: note = Description of Artificial Sequence: Syntheticpeptide
QQSYSTTAWT

SEQ ID NO: 262 - protein, 11 aa, synthetic construct
  REGION 1..11: note = Description of Artificial Sequence: Syntheticpeptide
RASQSIGQSLN

SEQ ID NO: 263 - protein, 7 aa, synthetic construct
  REGION 1..7: note = Description of Artificial Sequence: Syntheticpeptide
GASSLQS

SEQ ID NO: 264 - protein, 9 aa, synthetic construct
  REGION 1..9: note = Description of Artificial Sequence: Syntheticpeptide
QQSYSTPIT

SEQ ID NO: 265 - protein, 112 aa, synthetic construct
  REGION 1..112: note = Description of Artificial Sequence: Syntheticpolypeptide
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIK

SEQ ID NO: 266 - protein, 121 aa, synthetic construct
  REGION 1..121: note = Description of Artificial Sequence: Syntheticpolypeptide
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSS

SEQ ID NO: 267 - protein, 6 aa, synthetic construct
  REGION 1..6: note = Description of Artificial Sequence: Syntheticpeptide
SDYYMT

SEQ ID NO: 268 - protein, 7 aa, synthetic construct
  REGION 1..7: note = Description of Artificial Sequence: Syntheticpeptide
GFTFSDY

SEQ ID NO: 269 - protein, 10 aa, synthetic construct
  REGION 1..10: note = Description of Artificial Sequence: Syntheticpeptide
GFTFSDYYMT

SEQ ID NO: 270 - protein, 8 aa, synthetic construct
  REGION 1..8: note = Description of Artificial Sequence: Syntheticpeptide
RNRARGYT

SEQ ID NO: 271 - protein, 19 aa, synthetic construct
  REGION 1..19: note = Description of Artificial Sequence: Syntheticpeptide
FIRNRARGYTSDHNPSVKG

SEQ ID NO: 272 - protein, 10 aa, synthetic construct
  REGION 1..10: note = Description of Artificial Sequence: Syntheticpeptide
DRPSYYVLDY

SEQ ID NO: 273 - protein, 17 aa, synthetic construct
  REGION 1..17: note = Description of Artificial Sequence: Syntheticpeptide
KSSQSLFNVRSRKNYLA

SEQ ID NO: 274 - protein, 7 aa, synthetic construct
  REGION 1..7: note = Description of Artificial Sequence: Syntheticpeptide
WASTRES

SEQ ID NO: 275 - protein, 8 aa, synthetic construct
  REGION 1..8: note = Description of Artificial Sequence: Syntheticpeptide
KQSYDLFT

SEQ ID NO: 276 - DNA, 336 bp, synthetic construct
  misc_feature 1..336: note = Description of Artificial Sequence: Syntheticpolynucleotide
  source 1..336: mol_type = other DNA
gacattgtgatgactcaatcccccgactccctggctgtgtccctcggcgaacgcgcaactatcaactgtaaaagcagccagtccctgttcaacgtccggtcgaggaagaactacctggcctggtatcagcagaaacctgggcagccgccgaagcttctgatctcatgggcctcaactcgggaaagcggagtgccagatagattctccggatctggctccggaaccgacttcaccctgacgatttcgagcttgcaagcggaggatgtggccgtgtactactgcaagcagtcctacgacctcttcacctttggttcgggcaccaagctggagatcaaa

SEQ ID NO: 277 - DNA, 363 bp, synthetic construct
  misc_feature 1..363: note = Description of Artificial Sequence: Syntheticpolynucleotide
  source 1..363: mol_type = other DNA
gaagtccaacttgtcgaatcgggaggaggccttgtgcaacccggtggatccctgaggctgtcatgcgcggcctcgggcttcaccttttccgattactacatgacctgggtcagacaggcccctggaaaggggttggaatgggtggcattcatccggaatagagcccgcggatacacttccgaccacaaccccagcgtgaaggggcggttcaccattagccgcgacaacgccaagaactccctctacctccaaatgaacagcctgcgggcggaggataccgctgtgtactactgcgcccgcgaccggccgtcctactatgtgctggactactggggccagggtactacggtcaccgtctcctca

SEQ ID NO: 278 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 279 - protein, 255 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSD

SEQ ID NO: 280 - protein, 255 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGF

SEQ ID NO: 281 - protein, 255 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPS

SEQ ID NO: 282 - protein, 326 aa, synthetic construct
  REGION 1..326: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 283 - DNA, 255 bp, synthetic construct
  misc_feature 1..255: note = Description of Artificial Sequence: Syntheticpolynucleotide
  source 1..255: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtagtgaccgtgccctccagcaacttcggcacccagacctacacctgcaacgta

SEQ ID NO: 284 - protein, 326 aa, synthetic construct
  REGION 1..326: note = Description of Artificial Sequence: Syntheticpolypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 285 - DNA, 255 bp, synthetic construct; identical to SEQ ID NO: 283
  misc_feature 1..255: note = Description of Artificial Sequence: Syntheticpolynucleotide
  source 1..255: mol_type = other DNA

SEQ ID NO: 286 - protein, 107 aa, Homo sapiens
GTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - DNA, 255 bp, synthetic construct
  misc_feature 1..255: note = Description of Artificial Sequence: Syntheticpolynucleotide
  source 1..255: mol_type = other DNA
ggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctac

SEQ ID NO: 288 - protein, 107 aa, synthetic construct
  REGION 1..107: note = Description of Artificial Sequence: Syntheticpolypeptide
DIQMTQSPSTLSASVGDRVTITCRASQSISTWLAWYQQKPGKAPKVLIYKASSLESGVPSRFSGSGSGTEFILTINSLQPDDFASYYCQQYKSYSHTFGQGTKLEIK

SEQ ID NO: 289 - protein, 118 aa, synthetic construct
  REGION 1..118: note = Description of Artificial Sequence: Syntheticpolypeptide
QVQLQESGPGLVKPSETLSLTCTVSGGSISYYYWTWIRQPPGKGLEWIGHIYYSGSTNYNPSLKSRVTISIDTSKNLFSLKLSSVTAADTAVYYCARAEGSLDAFDFWGQGTMVTVSS

SEQ ID NO: 290 - protein, 107 aa, synthetic construct
  REGION 1..107: note = Description of Artificial Sequence: Syntheticpolypeptide
DIQMTQSPSTLSASVGDRVTITCRASQSISSWLAWYQQKPGKAPKVLIYKASSLESGVPSRFSGSGSGTEFTLTINSLQPDDFATYYCQQYKSFSLTFGQGTKLEIK

SEQ ID NO: 291 - protein, 113 aa, synthetic construct
  REGION 1..113: note = Description of Artificial Sequence: Syntheticpolypeptide
EVQLVESGGGLIQPGGSLRLSCAASGFTVSSNYMTWVRQAPGKGLEWVSVIYSGGSTYYADSVKGRFTISRDNSKNTLYLQMNSLRAEDTAVYYCARNWGDYWGQGTLVTVSS

SEQ ID NO: 292 - protein, 107 aa, synthetic construct
  REGION 1..107: note = Description of Artificial Sequence: Syntheticpolypeptide
DIQMTQSPSTLSASVGDRVTITCRASQSISSWLAWYQQKPGKAPKVLIYKASTLESGVPSRFSGSGSGTEFTLTISSLQPDDFATYYCQQYNSYSYTFGHGTKLEIK

SEQ ID NO: 293 - protein, 118 aa, synthetic construct
  REGION 1..118: note = Description of Artificial Sequence: Syntheticpolypeptide
QVQLQESGPGLVKPSGTLSLTCTVSGGSIDYYFWNWFRQSPVKGLEWIGHVYDIGNTKYNPSLKSRVTISIDTSENQFSLKLNSVTAADTAVYYCARGEGALDAFDIWGQGTMVTVSS

SEQ ID NO: 294 - protein, 107 aa, synthetic construct
  REGION 1..107: note = Description of Artificial Sequence: Syntheticpolypeptide
DIQMTQSPSILSASVGDRVTITCRASQSVSSWLAWYQQKPGKAPKVLIYKASSLESGVPSRFSGTGSGTEFTLTISSLQSDDFATYYCQQYNTYSHTFGQGTKLEIK

SEQ ID NO: 295 - protein, 118 aa, synthetic construct
  REGION 1..118: note = Description of Artificial Sequence: Syntheticpolypeptide
QVQLQESGPGLVKPSETLSLNCTVSGGSISYYYWTWIRQPPGKGLEWIGHVIYSGTTNYNPSLKSRVTISVDTSKNQFSLKLNSVTAADTAVYYCVRAEGSLDAFDLWGQGTMVTVSS

SEQ ID NO: 296 - protein, 107 aa, synthetic construct
  REGION 1..107: note = Description of Artificial Sequence: Syntheticpolypeptide
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPFTFGPGTKVDIK

SEQ ID NO: 297 - protein, 120 aa, synthetic construct
  REGION 1..120: note = Description of Artificial Sequence: Syntheticpolypeptide
QVQLVESGGGVVQPGRSLRLSCVASGFTFSDYGIHWVRQAPGMGQEWVAVIWYDGSIKKYSDSVKGRFIISRDNSENTVYLQMNSLRGEDTAIYYCARDEVGTFGAFDFWGQGTKVTVSS

SEQ ID NO: 298 - protein, 107 aa, synthetic construct
  REGION 1..107: note = Description of Artificial Sequence: Syntheticpolypeptide
DIQMTQSPSSLSASVGDSITITCRASQDIDNYLAWYQQKTGKVPKVLIYAASALQSGVPSRFSGSGSGTDFTLTISSLQPEDVATYYCQNYNSGPRTFGQGTKVEIK

SEQ ID NO: 299 - protein, 118 aa, synthetic construct
  REGION 1..118: note = Description of Artificial Sequence: Syntheticpolypeptide
QVQLQESGPGLVKPLQTLSLTCTVSGGSISSDGYYWSWIRQNPGKGLEWIGYMYYSGSTYYNPSLKSRVTISVDTSKNQFSLKLRSVTAADTAVYYCTRDFGWYFDLWGRGTLVTVSS

SEQ ID NO: 300 - protein, 107 aa, synthetic construct
  REGION 1..107: note = Description of Artificial Sequence: Syntheticpolypeptide
DIQMTQSPSSLSASVGDRVTITCRASQDISNYLTWYQQKPGRVPEVLIYAASALQSGVPSRFSGSGSGTDFTLTISSLQPEDVATYYCQNYNSAPRTFGQGTKVEIK

SEQ ID NO: 301 - protein, 118 aa, synthetic construct
  REGION 1..118: note = Description of Artificial Sequence: Syntheticpolypeptide
QVQLQESGPGLVKPSQSLSLTCSVSGGSVSSDGYYWSWIRQHPGKGLEWIGYIYYRRITDYNPSLKSRVNISLDTSKNQFSLKLSSVTAADTAVYYCARDFGWYFDLWGRGTLVAVSS

SEQ ID NO: 302 - protein, 112 aa, synthetic construct
  REGION 1..112: note = Description of Artificial Sequence: Syntheticpolypeptide
DIVMTQSPLSLPVTPGEPASISCRSSQSLLHSNGYNYLDWYLQKPGQSPQVLILLGSNRASGVPDRVSASGSGTDFTLKISRMQAEDVGIYYCMQTLQTPFTFGQGTKLEIK

SEQ ID NO: 303 - protein, 122 aa, synthetic construct
  REGION 1..122: note = Description of Artificial Sequence: Syntheticpolypeptide
QVQLVQSGAEVKKPGASVKVSCKASGYTFTGYYLHWVRQAPGQGLEWMGWINPNSGGTNYAQKFQGRVTMTRDTSITTAYMELSRLRIDDTAVYYCARDRGVTMIVDGMDDWGQGTTVTVSS

SEQ ID NO: 304 - protein, 107 aa, synthetic construct
  REGION 1..107: note = Description of Artificial Sequence: Syntheticpolypeptide
DIQLTQSPSFLSASVGDRVIITCRASQGINSHLAWYQQKPGKAPKLLIYYASTLPSGVPSRFSGSGSGTEFTLTVTSLQPEDFATYYCQQLNHYPITFGQGTRLDIN

SEQ ID NO: 305 - protein, 120 aa, synthetic construct
  REGION 1..120: note = Description of Artificial Sequence: Syntheticpolypeptide
EVELVESGGGMVQPGRSLRLSCAASGFTFSDYGMHWVRQAPGMGLEWVTVIWYDGSNKYYADSVKGRFTISRDNSKNTVFLQMNSLRAEDTAVYYCARDEVGFVGAFDIWGQGTMVTVSS

SEQ ID NO: 306 - protein, 107 aa, synthetic construct
  REGION 1..107: note = Description of Artificial Sequence: Syntheticpolypeptide
DIQMTQSPSSLSASVGDRVTITCRASQGISNYLAWYQQKPGKVPKLLIYAASTLHSGVPSRFSGSGSGTDFTLTISSLQPEDVATYYCQKYNSAPRTFGQGTKVEIK

SEQ ID NO: 307 - protein, 118 aa, synthetic construct
  REGION 1..118: note = Description of Artificial Sequence: Syntheticpolypeptide
QVQLQESGPGLVKPSQTLSLTCTVSGGSISSDGYYWSWIRQHPGKGLEWIGYMYYSGITYHNPSLKSRVTISVDTSKNQFSLRLSSVTAADTAVYYCARDFGWYFDLWGRGTLVTVSS

SEQ ID NO: 308 - protein, 107 aa, synthetic construct
  REGION 1..107: note = Description of Artificial Sequence: Syntheticpolypeptide
DIQMTQSPSSLSASVGDRVTITCRASQDISNYLAWYQQKPGKVPKLLIYAASTLHSGVPSRFSGSGSGTDFTLTISSLQPDDFAAYYCQKYNSAPRTFGQGTKVEIK

SEQ ID NO: 309 - protein, 118 aa, synthetic construct
  REGION 1..118: note = Description of Artificial Sequence: Syntheticpolypeptide
QVQLQESGPGLVKPSQTLSLTCTVSGGSISSDAYYWSWIRQHPGKGLEWIGYMYYSGITYYNPSLKSRVTISVDTSKNQFSLKLSSVTAADTAVYYCARDFGWYFDLWGRGTLVTVSS

SEQ ID NO: 310 - protein, 107 aa, synthetic construct
  REGION 1..107: note = Description of Artificial Sequence: Syntheticpolypeptide
DIQLTQSPSFLSASVGDRVSITCRASQDISSFLAWYQQKPGKAPVLLIYVASTLQSGVPSRFSGSGSGTEFTLTVSSLQPEDFATYYCQQLHVYPITFGQGTRLEIR

SEQ ID NO: 311 - protein, 120 aa, synthetic construct; identical to SEQ ID NO: 297
  REGION 1..120: note = Description of Artificial Sequence: Syntheticpolypeptide

SEQ ID NO: 312 - protein, 113 aa, synthetic construct
  REGION 1..113: note = Description of Artificial Sequence: Syntheticpolypeptide
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPFTFGPGTKVDIK

SEQ ID NO: 313 - protein, 122 aa, synthetic construct
  REGION 1..122: note = Description of Artificial Sequence: Syntheticpolypeptide
EVQLVESGGGLVKPGGSLRLSCAASGFTFSNAWMSWVRQAPGKGLEWVGRIKSKTGGGTTDYAAPVKGRFTISRDDSKNTLYLQMNSLKTEDTAVYYCTSLIVGAISLFDYWGQGTLVTVSS

SEQ ID NO: 314 - protein, 107 aa, synthetic construct
  REGION 1..107: note = Description of Artificial Sequence: Syntheticpolypeptide
DIQMTQSPSALSASVGDRVAITCRASQTISTWLAWYQQKPGKAPKVLIYKASNLESGVPSRFSGSGSGTEFTLTINSLQPDDFATYYCQQYQTFSHTFGQGTKLEIK

SEQ ID NO: 315 - protein, 118 aa, synthetic construct
  REGION 1..118: note = Description of Artificial Sequence: Syntheticpolypeptide
QVQLRESGPGLVKPSETLSLTCTISGGSISYYFWTWIRQPPGRGLEWIGQIYYSGNTNSNPSLKSRVTISIDTSKNQFSLKLTSVTVADTAVYYCVRAEGSLDAFDIWGQGTMVAVSS

SEQ ID NO: 316 - protein, 107 aa, synthetic construct
  REGION 1..107: note = Description of Artificial Sequence: Syntheticpolypeptide
DMQMTQSPSSLSASVGDRVTLTCRASQGISNYLAWFQLKPGKVPKLLIYAASTLQSGVPSRFSGSGSGTDFALTISSLQPEDVATYYCQKYNSAPLTFGGGTKVEIK

SEQ ID NO: 317 - protein, 122 aa, synthetic construct
  REGION 1..122: note = Description of Artificial Sequence: Syntheticpolypeptide
EVQLVESGGGLVKPGGSLRLSCVASGFTFSSYSMNWVRQFPGKGLEWVSSISTSSNYIHYADSLQGRFTISRDNAKNSLYLQMSSLRVEDTAVYYCVRDKGTTLTNWYFDLWGRGTLVTVSS

SEQ ID NO: 318 - protein, 112 aa, synthetic construct
  REGION 1..112: note = Description of Artificial Sequence: Syntheticpolypeptide
DIVMTQSPLSLPVTPGEPASISCRSSQTLVHSNGYNYLNWYLQKPGQSPQLLIYLGSNRASGVPDRFSGSGSGSDFTLKISRMEAEDVGVYYCMQAIQTPYTFGQGTNVEIK

SEQ ID NO: 319 - protein, 121 aa, synthetic construct
  REGION 1..121: note = Description of Artificial Sequence: Syntheticpolypeptide
QVQLVESGGGVVQPGRSLRLSCGASGFTFSSYGMHWVRQAPGKGLEWVAVIWYDGSNKYYADSLKGRFTISRDNSKNTLYLQMNSLRAEDTAVYYCARDGYSGSSDAFDIWGQGTMVTVSS

SEQ ID NO: 320 - protein, 107 aa, synthetic construct
  REGION 1..107: note = Description of Artificial Sequence: Syntheticpolypeptide
DIQMTQSPSTLSASVGDRVTITCRASQSISSWLAWYQQKPGKAPKVLIYKASNLESGVPSRFSGSGSGTEFTLTISSLQPDDFATYYCQQYNSYSCTFGQGTKLEIK

SEQ ID NO: 321 - protein, 118 aa, synthetic construct
  REGION 1..118: note = Description of Artificial Sequence: Syntheticpolypeptide
QVQLQESGPGLVQPSETLSLTCTVSGGSISYYYWSWIRQPPGKGLEWIGNINYMGNTIYNPSLKSRVTISVDTSKDQFSLKLTSVSAADTAVYYCVRAEGSLDAFDFWGQGTLVAVSL

SEQ ID NO: 322 - protein, 107 aa, synthetic construct
  REGION 1..107: note = Description of Artificial Sequence: Syntheticpolypeptide
DIQMTQSPSSLSASVGDRIIFTCQASQDINNYLHWYQQKPGKAPKLLIYDASDWETGVPSRFSGSGSGTDFTFTISSLQPEDIATYYCQQYDHLPITFGQGTRVEIK

SEQ ID NO: 323 - protein, 123 aa, synthetic construct
  REGION 1..123: note = Description of Artificial Sequence: Syntheticpolypeptide
QVQLVQSGAEVTKPGASVKVSCKASGYIFTGYYIYWVRQAPGQGLEWMGWINPSSGGTNYAQKFQGRVTMARDTSISTAYMELSSLRSDDTAVYYCARDRKREYYYNFGMDVWGQGTTVTVST

SEQ ID NO: 324 - protein, 107 aa, synthetic construct
  REGION 1..107: note = Description of Artificial Sequence: Syntheticpolypeptide
DIQMTQSPSSLSASVGDRVILTCQASQDISNYLHWYQQKPGKAPKLLIYDASDLETGVPSRFSGSGSGADFTFTISNLQPEDFATYYCQQYDHLPITFGQGTRLEIK

SEQ ID NO: 325 - protein, 123 aa, synthetic construct
  REGION 1..123: note = Description of Artificial Sequence: Syntheticpolypeptide
QVQLVQSGAEVTKPGASVKVSCKASGYTFTSHYIYWVRQAPGQGLEWMGWINPNSGGTNYAQKFQDRVTMARDTSISTAYMELSRLRSDDTAVYYCAKDRKREYYYNFGMDVWGQGTTVTVSA

SEQ ID NO: 326 - protein, 107 aa, synthetic construct; identical to SEQ ID NO: 324
  REGION 1..107: note = Description of Artificial Sequence: Syntheticpolypeptide

SEQ ID NO: 327 - protein, 123 aa, synthetic construct
  REGION 1..123: note = Description of Artificial Sequence: Syntheticpolypeptide
QVQLVQFGVEVRKPGASVKVSCKVSGFTFTSHYIYWVRQAPGQGLEWMGWINPNSGGTKYAQKFQDRVTMARDTSISTAYMELSRLRSDDTSVYYCVKDRKREYYYNFGMDVWGQGTTVTVSS

SEQ ID NO: 328 - protein, 107 aa, synthetic construct
  REGION 1..107: note = Description of Artificial Sequence: Syntheticpolypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 329 - protein, 119 aa, synthetic construct
  REGION 1..119: note = Description of Artificial Sequence: Syntheticpolypeptide
EVQMLESGGGLIQPGGSLRLSCKTSGFTLSIYAIHWVRQAPGRGLEWVSSFGGRGSSTYFADSVKGRFTISRDASENSLYLHMNSLRAEDTAVYYCAKEKDWGRGFDYWGQGTLVTVSS

SEQ ID NO: 330 - protein, 112 aa, synthetic construct
  REGION 1..112: note = Description of Artificial Sequence: Syntheticpolypeptide
DIVMTQSPLSLPVTPGEPASISCRSSQSLLYSNGYNYLDWYLQKPGQSPQLLIFLNSNRASGVPDRFSGSGSGTDFTLKISRVEAEDVGVYFCMQALQTPLTFGGGTKVEIK

SEQ ID NO: 331 - protein, 122 aa, synthetic construct
  REGION 1..122: note = Description of Artificial Sequence: Syntheticpolypeptide
EVQLVESGGGLVKPGGSLRLSCAASGFTFSNYSMNWVRQAPGKGLEWVSSISSSTIYIYYADSVKGRFTISRDNAKKSLYLQMNSLRAEDTAVYYCARDIGWEVFTLGFDYWGQGTQVTVSS

SEQ ID NO: 332 - protein, 5 aa, synthetic construct
  REGION 1..5: note = Description of Artificial Sequence: Syntheticpeptide
YYYWT

SEQ ID NO: 333 - protein, 7 aa, synthetic construct
  REGION 1..7: note = Description of Artificial Sequence: Syntheticpeptide
GGSISYY

SEQ ID NO: 334 - protein, 10 aa, synthetic construct
  REGION 1..10: note = Description of Artificial Sequence: Syntheticpeptide
GGSISYYYWT

SEQ ID NO: 335 - protein, 16 aa, synthetic construct
  REGION 1..16: note = Description of Artificial Sequence: Syntheticpeptide
HIYYSGSTNYNPSLKS

SEQ ID NO: 336 - protein, 5 aa, synthetic construct
  REGION 1..5: note = Description of Artificial Sequence: Syntheticpeptide
YYSGS

SEQ ID NO: 337 - protein, 10 aa, synthetic construct
  REGION 1..10: note = Description of Artificial Sequence: Syntheticpeptide
AEGSLDAFDF

SEQ ID NO: 338 - protein, 5 aa, synthetic construct
  REGION 1..5: note = Description of Artificial Sequence: Syntheticpeptide
SNYMT

SEQ ID NO: 339 - protein, 7 aa, synthetic construct
  REGION 1..7: note = Description of Artificial Sequence: Syntheticpeptide
GFTVSSN

SEQ ID NO: 340 - protein, 10 aa, synthetic construct
  REGION 1..10: note = Description of Artificial Sequence: Syntheticpeptide
GFTVSSNYMT

SEQ ID NO: 341 - protein, 16 aa, synthetic construct
  REGION 1..16: note = Description of Artificial Sequence: Syntheticpeptide
VIYSGGSTYYADSVKG

SEQ ID NO: 342 - protein, 5 aa, synthetic construct
  REGION 1..5: note = Description of Artificial Sequence: Syntheticpeptide
YSGGS

SEQ ID NO: 343 - protein, 6 aa, synthetic construct
  REGION 1..6: note = Description of Artificial Sequence: Syntheticpeptide
NWGDYW

SEQ ID NO: 344 - protein, 5 aa, synthetic construct
  REGION 1..5: note = Description of Artificial Sequence: Syntheticpeptide
YYFWN

SEQ ID NO: 345 - protein, 7 aa, synthetic construct
  REGION 1..7: note = Description of Artificial Sequence: Syntheticpeptide
GGSIDYY

SEQ ID NO: 346 - protein, 10 aa, synthetic construct
  REGION 1..10: note = Description of Artificial Sequence: Syntheticpeptide
GGSIDYYFWN

SEQ ID NO: 347 - protein, 16 aa, synthetic construct
  REGION 1..16: note = Description of Artificial Sequence: Syntheticpeptide
HVYDIGNTKYNPSLKS

SEQ ID NO: 348 - protein, 5 aa, synthetic construct
  REGION 1..5: note = Description of Artificial Sequence: Syntheticpeptide
YDIGN

SEQ ID NO: 349 - protein, 10 aa, synthetic construct
  REGION 1..10: note = Description of Artificial Sequence: Syntheticpeptide
GEGALDAFDI

SEQ ID NO: 350 - protein, 5 aa, synthetic construct
  REGION 1..5: note = Description of Artificial Sequence: Syntheticpeptide
YYYWT

SEQ ID NO: 351 - protein, 7 aa, synthetic construct
  REGION 1..7: note = Description of Artificial Sequence: Syntheticpeptide
GGSISYY

SEQ ID NO: 352 - protein, 10 aa, synthetic construct
  REGION 1..10: note = Description of Artificial Sequence: Syntheticpeptide
GGSISYYYWT

SEQ ID NO: 353 - protein, 16 aa, synthetic construct
  REGION 1..16: note = Description of Artificial Sequence: Syntheticpeptide
HVIYSGTTNYNPSLKS

SEQ ID NO: 354 - protein, 5 aa, synthetic construct
  REGION 1..5: note = Description of Artificial Sequence: Syntheticpeptide
IYSGT

SEQ ID NO: 355 - protein, 10 aa, synthetic construct
  REGION 1..10: note = Description of Artificial Sequence: Syntheticpeptide
AEGSLDAFDL

SEQ ID NO: 356 - protein, 5 aa, synthetic construct
  REGION 1..5: note = Description of Artificial Sequence: Syntheticpeptide
DYGIH

SEQ ID NO: 357 - protein, 7 aa, synthetic construct
  REGION 1..7: note = Description of Artificial Sequence: Syntheticpeptide
GFTFSDY

SEQ ID NO: 358 - protein, 10 aa, synthetic construct
  REGION 1..10: note = Description of Artificial Sequence: Syntheticpeptide
GFTFSDYGIH

SEQ ID NO: 359 - protein, 17 aa, synthetic construct
  REGION 1..17: note = Description of Artificial Sequence: Syntheticpeptide
VIWYDGSIKKYSDSVKG

SEQ ID NO: 360 - protein, 6 aa, synthetic construct
  REGION 1..6: note = Description of Artificial Sequence: Syntheticpeptide
WYDGSI

SEQ ID NO: 361 - protein, 11 aa, synthetic construct
  REGION 1..11: note = Description of Artificial Sequence: Syntheticpeptide
DEVGTFGAFDF

SEQ ID NO: 362 - protein, 7 aa, synthetic construct
  REGION 1..7: note = Description of Artificial Sequence: Syntheticpeptide
SDGYYWS

SEQ ID NO: 363 - protein, 9 aa, synthetic construct
  REGION 1..9: note = Description of Artificial Sequence: Syntheticpeptide
GGSISSDGY

SEQ ID NO: 364 - protein, 12 aa, synthetic construct
  REGION 1..12: note = Description of Artificial Sequence: Syntheticpeptide
GGSISSDGYYWS

SEQ ID NO: 365 - protein, 16 aa, synthetic construct
  REGION 1..16: note = Description of Artificial Sequence: Syntheticpeptide
YMYYSGSTYYNPSLKS

SEQ ID NO: 366 - protein, 5 aa, synthetic construct
  REGION 1..5: note = Description of Artificial Sequence: Syntheticpeptide
YYSGS

SEQ ID NO: 367 - protein, 8 aa, synthetic construct
  REGION 1..8: note = Description of Artificial Sequence: Syntheticpeptide
DFGWYFDL

SEQ ID NO: 368 - protein, 7 aa, synthetic construct
  REGION 1..7: note = Description of Artificial Sequence: Syntheticpeptide
SDGYYWS

SEQ ID NO: 369 - protein, 9 aa, synthetic construct
  REGION 1..9: note = Description of Artificial Sequence: Syntheticpeptide
GGSVSSDGY

SEQ ID NO: 370 - protein, 12 aa, synthetic construct
  REGION 1..12: note = Description of Artificial Sequence: Syntheticpeptide
GGSVSSDGYYWS

SEQ ID NO: 371 - protein, 16 aa, synthetic construct
  REGION 1..16: note = Description of Artificial Sequence: Syntheticpeptide
YIYYRRITDYNPSLKS

SEQ ID NO: 372 - protein, 5 aa, synthetic construct
  REGION 1..5: note = Description of Artificial Sequence: Syntheticpeptide
YYRRI

SEQ ID NO: 373 - protein, 8 aa, synthetic construct
  REGION 1..8: note = Description of Artificial Sequence: Syntheticpeptide
DFGWYFDL

SEQ ID NO: 374 - protein, 5 aa, synthetic construct
  REGION 1..5: note = Description of Artificial Sequence: Syntheticpeptide
GYYLH

SEQ ID NO: 375 - protein, 7 aa, synthetic construct
  REGION 1..7: note = Description of Artificial Sequence: Syntheticpeptide
GYTFTGY

SEQ ID NO: 376 - protein, 10 aa, synthetic construct
  REGION 1..10: note = Description of Artificial Sequence: Syntheticpeptide
GYTFTGYYLH

SEQ ID NO: 377 - protein, 17 aa, synthetic construct
  REGION 1..17: note = Description of Artificial Sequence: Syntheticpeptide
WINPNSGGTNYAQKFQG

SEQ ID NO: 378 - protein, 6 aa, synthetic construct
  REGION 1..6: note = Description of Artificial Sequence: Syntheticpeptide
NPNSGG

SEQ ID NO: 379 - protein, 13 aa, synthetic construct
  REGION 1..13: note = Description of Artificial Sequence: Syntheticpeptide
DRGVTMIVDGMDD

SEQ ID NO: 380 - protein, 5 aa, synthetic construct
  REGION 1..5: note = Description of Artificial Sequence: Syntheticpeptide
DYGMH

SEQ ID NO: 381 - protein, 7 aa, synthetic construct
  REGION 1..7: note = Description of Artificial Sequence: Syntheticpeptide
GFTFSDY

SEQ ID NO: 382 - protein, 10 aa, synthetic construct
  REGION 1..10: note = Description of Artificial Sequence: Syntheticpeptide
GFTFSDYGMH

SEQ ID NO: 383 - protein, 17 aa, synthetic construct
  REGION 1..17: note = Description of Artificial Sequence: Syntheticpeptide
VIWYDGSNKYYADSVKG

SEQ ID NO: 384 - protein, 6 aa, synthetic construct
  REGION 1..6: note = Description of Artificial Sequence: Syntheticpeptide
WYDGSN

SEQ ID NO: 385 - protein, 11 aa, synthetic construct
  REGION 1..11: note = Description of Artificial Sequence: Syntheticpeptide
DEVGFVGAFDI

SEQ ID NO: 386 - protein, 7 aa, synthetic construct
  REGION 1..7: note = Description of Artificial Sequence: Syntheticpeptide
SDGYYWS

SEQ ID NO: 387 - protein, 9 aa, synthetic construct
  REGION 1..9: note = Description of Artificial Sequence: Syntheticpeptide
GGSISSDGY

SEQ ID NO: 388 - protein, 12 aa, synthetic construct
  REGION 1..12: note = Description of Artificial Sequence: Syntheticpeptide
GGSISSDGYYWS

SEQ ID NO: 389 - protein, 16 aa, synthetic construct
  REGION 1..16: note = Description of Artificial Sequence: Syntheticpeptide
YMYYSGITYHNPSLKS

SEQ ID NO: 390 - protein, 5 aa, synthetic construct
  REGION 1..5: note = Description of Artificial Sequence: Syntheticpeptide
YYSGI

SEQ ID NO: 391 - protein, 8 aa, synthetic construct
  REGION 1..8: note = Description of Artificial Sequence: Syntheticpeptide
DFGWYFDL

SEQ ID NO: 392 - protein, 7 aa, synthetic construct
  REGION 1..7: note = Description of Artificial Sequence: Syntheticpeptide
SDAYYWS

SEQ ID NO: 393 - protein, 9 aa, synthetic construct
  REGION 1..9: note = Description of Artificial Sequence: Syntheticpeptide
GGSISSDAY

SEQ ID NO: 394 - protein, 12 aa, synthetic construct
  REGION 1..12: note = Description of Artificial Sequence: Syntheticpeptide
GGSISSDAYYWS

SEQ ID NO: 395 - protein, 16 aa, synthetic construct
  REGION 1..16: note = Description of Artificial Sequence: Syntheticpeptide
YMYYSGITYYNPSLKS

SEQ ID NO: 396 - protein, 5 aa, synthetic construct
  REGION 1..5: note = Description of Artificial Sequence: Syntheticpeptide
YYSGI

SEQ ID NO: 397 - protein, 8 aa, synthetic construct
  REGION 1..8: note = Description of Artificial Sequence: Syntheticpeptide
DFGWYFDL

SEQ ID NO: 398 - protein, 5 aa, synthetic construct
  REGION 1..5: note = Description of Artificial Sequence: Syntheticpeptide
DYGIH

SEQ ID NO: 399 - protein, 7 aa, synthetic construct
  REGION 1..7: note = Description of Artificial Sequence: Syntheticpeptide
GFTFSDY

SEQ ID NO: 400 - protein, 10 aa, synthetic construct
  REGION 1..10: note = Description of Artificial Sequence: Syntheticpeptide
GFTFSDYGIH

SEQ ID NO: 401 - protein, 17 aa, synthetic construct
  REGION 1..17: note = Description of Artificial Sequence: Syntheticpeptide
VIWYDGSIKKYSDSVKG

SEQ ID NO: 402 - protein, 6 aa, synthetic construct
  REGION 1..6: note = Description of Artificial Sequence: Syntheticpeptide
WYDGSI

SEQ ID NO: 403 - protein, 11 aa, synthetic construct
  REGION 1..11: note = Description of Artificial Sequence: Syntheticpeptide
DEVGTFGAFDF

SEQ ID NO: 404 - protein, 5 aa, synthetic construct
  REGION 1..5: note = Description of Artificial Sequence: Syntheticpeptide
NAWMS

SEQ ID NO: 405 - protein, 7 aa, synthetic construct
  REGION 1..7: note = Description of Artificial Sequence: Syntheticpeptide
GFTFSNA

SEQ ID NO: 406 - protein, 10 aa, synthetic construct
  REGION 1..10: note = Description of Artificial Sequence: Syntheticpeptide
GFTFSNAWMS

SEQ ID NO: 407 - protein, 19 aa, synthetic construct
  REGION 1..19: note = Description of Artificial Sequence: Syntheticpeptide
RIKSKTGGGTTDYAAPVKG

SEQ ID NO: 408 - protein, 8 aa, synthetic construct
  REGION 1..8: note = Description of Artificial Sequence: Syntheticpeptide
KSKTGGGT

SEQ ID NO: 409 - protein, 11 aa, synthetic construct
  REGION 1..11: note = Description of Artificial Sequence: Syntheticpeptide
LIVGAISLFDY

SEQ ID NO: 410 - protein, 5 aa, synthetic construct
  REGION 1..5: note = Description of Artificial Sequence: Syntheticpeptide
YYFWT

SEQ ID NO: 411 - protein, 7 aa, synthetic construct
  REGION 1..7: note = Description of Artificial Sequence: Syntheticpeptide
GGSISYY

SEQ ID NO: 412 - protein, 10 aa, synthetic construct
  REGION 1..10: note = Description of Artificial Sequence: Syntheticpeptide
GGSISYYFWT

SEQ ID NO: 413 - protein, 16 aa, synthetic construct
  REGION 1..16: note = Description of Artificial Sequence: Syntheticpeptide
QIYYSGNTNSNPSLKS

SEQ ID NO: 414 - protein, 5 aa, synthetic construct
  REGION 1..5: note = Description of Artificial Sequence: Syntheticpeptide
YYSGN

SEQ ID NO: 415 - protein, 10 aa, synthetic construct
  REGION 1..10: note = Description of Artificial Sequence: Syntheticpeptide
AEGSLDAFDI

SEQ ID NO: 416 - protein, 5 aa, synthetic construct
  REGION 1..5: note = Description of Artificial Sequence: Syntheticpeptide
SYSMN

SEQ ID NO: 417 - protein, 7 aa, synthetic construct
  REGION 1..7: note = Description of Artificial Sequence: Syntheticpeptide
GFTFSSY

SEQ ID NO: 418 - protein, 10 aa, synthetic construct
  REGION 1..10: note = Description of Artificial Sequence: Syntheticpeptide
GFTFSSYSMN

SEQ ID NO: 419 - protein, 17 aa, synthetic construct
  REGION 1..17: note = Description of Artificial Sequence: Syntheticpeptide
SISTSSNYIHYADSLQG

SEQ ID NO: 420 - protein, 6 aa, synthetic construct
  REGION 1..6: note = Description of Artificial Sequence: Syntheticpeptide
STSSNY

SEQ ID NO: 421 - protein, 13 aa, synthetic construct
  REGION 1..13: note = Description of Artificial Sequence: Syntheticpeptide
DKGTTLTNWYFDL

SEQ ID NO: 422 - protein, 5 aa, synthetic construct
  REGION 1..5: note = Description of Artificial Sequence: Syntheticpeptide
SYGMH

SEQ ID NO: 423 - protein, 7 aa, synthetic construct
  REGION 1..7: note = Description of Artificial Sequence: Syntheticpeptide
GFTFSSY

SEQ ID NO: 424 - protein, 10 aa, synthetic construct
  REGION 1..10: note = Description of Artificial Sequence: Syntheticpeptide
GFTFSSYGMH

SEQ ID NO: 425 - protein, 17 aa, synthetic construct
  REGION 1..17: note = Description of Artificial Sequence: Syntheticpeptide
VIWYDGSNKYYADSLKG

SEQ ID NO: 426 - protein, 6 aa, synthetic construct
  REGION 1..6: note = Description of Artificial Sequence: Syntheticpeptide
WYDGSN

SEQ ID NO: 427 - protein, 12 aa, synthetic construct
  REGION 1..12: note = Description of Artificial Sequence: Syntheticpeptide
DGYSGSSDAFDI

SEQ ID NO: 428 - protein, 5 aa, synthetic construct
  REGION 1..5: note = Description of Artificial Sequence: Syntheticpeptide
YYYWS

SEQ ID NO: 429 - protein, 7 aa, synthetic construct
  REGION 1..7: note = Description of Artificial Sequence: Syntheticpeptide
GGSISYY

SEQ ID NO: 430 - protein, 10 aa, synthetic construct
  REGION 1..10: note = Description of Artificial Sequence: Syntheticpeptide
GGSISYYYWS

SEQ ID NO: 431 - protein, 16 aa, synthetic construct
  REGION 1..16: note = Description of Artificial Sequence: Syntheticpeptide
NINYMGNTIYNPSLKS

SEQ ID NO: 432 - protein, 5 aa, synthetic construct
  REGION 1..5: note = Description of Artificial Sequence: Syntheticpeptide
NYMGN

SEQ ID NO: 433 - protein, 10 aa, synthetic construct
  REGION 1..10: note = Description of Artificial Sequence: Syntheticpeptide
AEGSLDAFDF

SEQ ID NO: 434 - protein, 5 aa, synthetic construct
  REGION 1..5: note = Description of Artificial Sequence: Syntheticpeptide
GYYIY

SEQ ID NO: 435 - protein, 7 aa, synthetic construct
  REGION 1..7: note = Description of Artificial Sequence: Syntheticpeptide
GYIFTGY

SEQ ID NO: 436 - protein, 10 aa, synthetic construct
  REGION 1..10: note = Description of Artificial Sequence: Syntheticpeptide
GYIFTGYYIY

SEQ ID NO: 437 - protein, 17 aa, synthetic construct
  REGION 1..17: note = Description of Artificial Sequence: Syntheticpeptide
WINPSSGGTNYAQKFQG

SEQ ID NO: 438 - protein, 6 aa, synthetic construct
  REGION 1..6: note = Description of Artificial Sequence: Syntheticpeptide
NPSSGG

SEQ ID NO: 439 - protein, 14 aa, synthetic construct
  REGION 1..14: note = Description of Artificial Sequence: Syntheticpeptide
DRKREYYYNFGMDV

SEQ ID NO: 440 - protein, 5 aa, synthetic construct
  REGION 1..5: note = Description of Artificial Sequence: Syntheticpeptide
SHYIY

SEQ ID NO: 441 - protein, 7 aa, synthetic construct
  REGION 1..7: note = Description of Artificial Sequence: Syntheticpeptide
GYTFTSH

SEQ ID NO: 442 - protein, 10 aa, synthetic construct
  REGION 1..10: note = Description of Artificial Sequence: Syntheticpeptide
GYTFTSHYIY

SEQ ID NO: 443 - protein, 17 aa, synthetic construct
  REGION 1..17: note = Description of Artificial Sequence: Syntheticpeptide
WINPNSGGTNYAQKFQD

SEQ ID NO: 444 - protein, 6 aa, synthetic construct
  REGION 1..6: note = Description of Artificial Sequence: Syntheticpeptide
NPNSGG

SEQ ID NO: 445 - protein, 14 aa, synthetic construct
  REGION 1..14: note = Description of Artificial Sequence: Syntheticpeptide
DRKREYYYNFGMDV

SEQ ID NO: 446 - protein, 5 aa, synthetic construct
  REGION 1..5: note = Description of Artificial Sequence: Syntheticpeptide
SHYIY

SEQ ID NO: 447 - protein, 7 aa, synthetic construct
  REGION 1..7: note = Description of Artificial Sequence: Syntheticpeptide
GFTFTSH

SEQ ID NO: 448 - protein, 10 aa, synthetic construct
  REGION 1..10: note = Description of Artificial Sequence: Syntheticpeptide
GFTFTSHYIY

SEQ ID NO: 449 - protein, 17 aa, synthetic construct
  REGION 1..17: note = Description of Artificial Sequence: Syntheticpeptide
WINPNSGGTKYAQKFQD

SEQ ID NO: 450 - protein, 6 aa, synthetic construct
  REGION 1..6: note = Description of Artificial Sequence: Syntheticpeptide
NPNSGG

SEQ ID NO: 451 - protein, 14 aa, synthetic construct
  REGION 1..14: note = Description of Artificial Sequence: Syntheticpeptide
DRKREYYYNFGMDV

SEQ ID NO: 452 - protein, 5 aa, synthetic construct
  REGION 1..5: note = Description of Artificial Sequence: Syntheticpeptide
IYAIH

SEQ ID NO: 453 - protein, 7 aa, synthetic construct
  REGION 1..7: note = Description of Artificial Sequence: Syntheticpeptide
GFTLSIY

SEQ ID NO: 454 - protein, 10 aa, synthetic construct
  REGION 1..10: note = Description of Artificial Sequence: Syntheticpeptide
GFTLSIYAIH

SEQ ID NO: 455 - protein, 17 aa, synthetic construct
  REGION 1..17: note = Description of Artificial Sequence: Syntheticpeptide
SFGGRGSSTYFADSVKG

SEQ ID NO: 456 - protein, 6 aa, synthetic construct
  REGION 1..6: note = Description of Artificial Sequence: Syntheticpeptide
GGRGSS

SEQ ID NO: 457 - protein, 10 aa, synthetic construct
  REGION 1..10: note = Description of Artificial Sequence: Syntheticpeptide
EKDWGRGFDY

SEQ ID NO: 458 - protein, 5 aa, synthetic construct
  REGION 1..5: note = Description of Artificial Sequence: Syntheticpeptide
NYSMN

SEQ ID NO: 459 - protein, 7 aa, synthetic construct
  REGION 1..7: note = Description of Artificial Sequence: Syntheticpeptide
GFTFSNY

SEQ ID NO: 460 - protein, 10 aa, synthetic construct
  REGION 1..10: note = Description of Artificial Sequence: Syntheticpeptide
GFTFSNYSMN

SEQ ID NO: 461 - protein, 17 aa, synthetic construct
  REGION 1..17: note = Description of Artificial Sequence: Syntheticpeptide
SISSSTIYIYYADSVKG

SEQ ID NO: 462 - protein, 6 aa, synthetic construct
  REGION 1..6: note = Description of Artificial Sequence: Syntheticpeptide
SSSTIY

SEQ ID NO: 463 - protein, 13 aa, synthetic construct
  REGION 1..13: note = Description of Artificial Sequence: Syntheticpeptide
DIGWEVFTLGFDY

SEQ ID NO: 464 - protein, 11 aa, synthetic construct
  REGION 1..11: note = Description of Artificial Sequence: Syntheticpeptide
RASQSISTWLA

SEQ ID NO: 465 - protein, 7 aa, synthetic construct
  REGION 1..7: note = Description of Artificial Sequence: Syntheticpeptide
KASSLES

SEQ ID NO: 466 - protein, 9 aa, synthetic construct
  REGION 1..9: note = Description of Artificial Sequence: Syntheticpeptide
QQYKSYSHT

SEQ ID NO: 467 - protein, 11 aa, synthetic construct
  REGION 1..11: note = Description of Artificial Sequence: Syntheticpeptide
RASQSISSWLA

SEQ ID NO: 468 - protein, 7 aa, synthetic construct
  REGION 1..7: note = Description of Artificial Sequence: Syntheticpeptide
KASSLES

SEQ ID NO: 469 - protein, 9 aa, synthetic construct
  REGION 1..9: note = Description of Artificial Sequence: Syntheticpeptide
QQYKSFSLT

SEQ ID NO: 470 - protein, 11 aa, synthetic construct
  REGION 1..11: note = Description of Artificial Sequence: Syntheticpeptide
RASQSISSWLA

SEQ ID NO: 471 - protein, 7 aa, synthetic construct
  REGION 1..7: note = Description of Artificial Sequence: Syntheticpeptide
KASTLES

SEQ ID NO: 472 - protein, 9 aa, synthetic construct
  REGION 1..9: note = Description of Artificial Sequence: Syntheticpeptide
QQYNSYSYT

SEQ ID NO: 473 - protein, 11 aa, synthetic construct
  REGION 1..11: note = Description of Artificial Sequence: Syntheticpeptide
RASQSVSSWLA

SEQ ID NO: 474 - protein, 7 aa, synthetic construct
  REGION 1..7: note = Description of Artificial Sequence: Syntheticpeptide
KASSLES

SEQ ID NO: 475 - protein, 9 aa, synthetic construct
  REGION 1..9: note = Description of Artificial Sequence: Syntheticpeptide
QQYNTYSHT

SEQ ID NO: 476 - protein, 11 aa, synthetic construct
  REGION 1..11: note = Description of Artificial Sequence: Syntheticpeptide
RASQGIRNDLG

SEQ ID NO: 477 - protein, 7 aa, synthetic construct
  REGION 1..7: note = Description of Artificial Sequence: Syntheticpeptide
AASSLQS

SEQ ID NO: 478 - protein, 9 aa, synthetic construct
  REGION 1..9: note = Description of Artificial Sequence: Syntheticpeptide
LQDYNYPFT

SEQ ID NO: 479 - protein, 11 aa, synthetic construct
  REGION 1..11: note = Description of Artificial Sequence: Syntheticpeptide
RASQDIDNYLA

SEQ ID NO: 480 - protein, 7 aa, synthetic construct
  REGION 1..7: note = Description of Artificial Sequence: Syntheticpeptide
AASALQS

SEQ ID NO: 481 - protein, 9 aa, synthetic construct
  REGION 1..9: note = Description of Artificial Sequence: Syntheticpeptide
QNYNSGPRT

SEQ ID NO: 482 - protein, 11 aa, synthetic construct
  REGION 1..11: note = Description of Artificial Sequence: Syntheticpeptide
RASQDISNYLT

SEQ ID NO: 483 - protein, 7 aa, synthetic construct
  REGION 1..7: note = Description of Artificial Sequence: Syntheticpeptide
AASALQS

SEQ ID NO: 484 - protein, 9 aa, synthetic construct
  REGION 1..9: note = Description of Artificial Sequence: Syntheticpeptide
QNYNSAPRT

SEQ ID NO: 485 - protein, 16 aa, synthetic construct
  REGION 1..16: note = Description of Artificial Sequence: Syntheticpeptide
RSSQSLLHSNGYNYLD

SEQ ID NO: 486 - protein, 7 aa, synthetic construct
  REGION 1..7: note = Description of Artificial Sequence: Syntheticpeptide
LGSNRAS

SEQ ID NO: 487 - protein, 9 aa, synthetic construct
  REGION 1..9: note = Description of Artificial Sequence: Syntheticpeptide
MQTLQTPFT

SEQ ID NO: 488 - protein, 11 aa, synthetic construct
  REGION 1..11: note = Description of Artificial Sequence: Syntheticpeptide
RASQGINSHLA

SEQ ID NO: 489 - protein, 7 aa, synthetic construct
  REGION 1..7: note = Description of Artificial Sequence: Syntheticpeptide
YASTLPS

SEQ ID NO: 490 - protein, 9 aa, synthetic construct
  REGION 1..9: note = Description of Artificial Sequence: Syntheticpeptide
QQLNHYPIT

SEQ ID NO: 491 - protein, 11 aa, synthetic construct
  REGION 1..11: note = Description of Artificial Sequence: Syntheticpeptide
RASQGISNYLA

SEQ ID NO: 492 - protein, 7 aa, synthetic construct
  REGION 1..7: note = Description of Artificial Sequence: Syntheticpeptide
AASTLHS

SEQ ID NO: 493 - protein, 9 aa, synthetic construct
  REGION 1..9: note = Description of Artificial Sequence: Syntheticpeptide
QKYNSAPRT

SEQ ID NO: 494 - protein, 11 aa, synthetic construct
  REGION 1..11: note = Description of Artificial Sequence: Syntheticpeptide
RASQDISNYLA

SEQ ID NO: 495 - protein, 7 aa, synthetic construct
  REGION 1..7: note = Description of Artificial Sequence: Syntheticpeptide
AASTLHS

SEQ ID NO: 496 - protein, 9 aa, synthetic construct
  REGION 1..9: note = Description of Artificial Sequence: Syntheticpeptide
QKYNSAPRT

SEQ ID NO: 497 - protein, 11 aa, synthetic construct
  REGION 1..11: note = Description of Artificial Sequence: Syntheticpeptide
RASQDISSFLA

SEQ ID NO: 498 - protein, 7 aa, synthetic construct
  REGION 1..7: note = Description of Artificial Sequence: Syntheticpeptide
VASTLQS

SEQ ID NO: 499 - protein, 9 aa, synthetic construct
  REGION 1..9: note = Description of Artificial Sequence: Syntheticpeptide
QQLHVYPIT

SEQ ID NO: 500 - protein, 17 aa, synthetic construct
  REGION 1..17: note = Description of Artificial Sequence: Syntheticpeptide
RSSQSLLDSDDGNTYLD

SEQ ID NO: 501 - protein, 7 aa, synthetic construct
  REGION 1..7: note = Description of Artificial Sequence: Syntheticpeptide
TLSYRAS

SEQ ID NO: 502 - protein, 9 aa, synthetic construct
  REGION 1..9: note = Description of Artificial Sequence: Syntheticpeptide
MQRIEFPFT

SEQ ID NO: 503 - protein, 11 aa, synthetic construct
  REGION 1..11: note = Description of Artificial Sequence: Syntheticpeptide
RASQTISTWLA

SEQ ID NO: 504 - protein, 7 aa, synthetic construct
  REGION 1..7: note = Description of Artificial Sequence: Syntheticpeptide
KASNLES

SEQ ID NO: 505 - protein, 9 aa, synthetic construct
  REGION 1..9: note = Description of Artificial Sequence: Syntheticpeptide
QQYQTFSHT

SEQ ID NO: 506 - protein, 11 aa, synthetic construct
  REGION 1..11: note = Description of Artificial Sequence: Syntheticpeptide
RASQGISNYLA

SEQ ID NO: 507 - protein, 7 aa, synthetic construct
  REGION 1..7: note = Description of Artificial Sequence: Syntheticpeptide
AASTLQS

SEQ ID NO: 508 - protein, 9 aa, synthetic construct
  REGION 1..9: note = Description of Artificial Sequence: Syntheticpeptide
QKYNSAPLT

SEQ ID NO: 509 - protein, 16 aa, synthetic construct
  REGION 1..16: note = Description of Artificial Sequence: Syntheticpeptide
RSSQTLVHSNGYNYLN

SEQ ID NO: 510 - protein, 7 aa, synthetic construct
  REGION 1..7: note = Description of Artificial Sequence: Syntheticpeptide
LGSNRAS

SEQ ID NO: 511 - protein, 9 aa, synthetic construct
  REGION 1..9: note = Description of Artificial Sequence: Syntheticpeptide
MQAIQTPYT

SEQ ID NO: 512 - protein, 11 aa, synthetic construct
  REGION 1..11: note = Description of Artificial Sequence: Syntheticpeptide
RASQSISSWLA

SEQ ID NO: 513 - protein, 7 aa, synthetic construct
  REGION 1..7: note = Description of Artificial Sequence: Syntheticpeptide
KASNLES

SEQ ID NO: 514 - protein, 9 aa, synthetic construct
  REGION 1..9: note = Description of Artificial Sequence: Syntheticpeptide
QQYNSYSCT

SEQ ID NO: 515 - protein, 11 aa, synthetic construct
  REGION 1..11: note = Description of Artificial Sequence: Syntheticpeptide
QASQDINNYLH

SEQ ID NO: 516 - protein, 7 aa, synthetic construct
  REGION 1..7: note = Description of Artificial Sequence: Syntheticpeptide
DASDWET

SEQ ID NO: 517 - protein, 9 aa, synthetic construct
  REGION 1..9: note = Description of Artificial Sequence: Syntheticpeptide
QQYDHLPIT

SEQ ID NO: 518 - protein, 11 aa, synthetic construct
  REGION 1..11: note = Description of Artificial Sequence: Syntheticpeptide
QASQDISNYLH

SEQ ID NO: 519 - protein, 7 aa, synthetic construct
  REGION 1..7: note = Description of Artificial Sequence: Syntheticpeptide
DASDLET

SEQ ID NO: 520 - protein, 9 aa, synthetic construct
  REGION 1..9: note = Description of Artificial Sequence: Syntheticpeptide
QQYDHLPIT

SEQ ID NO: 521 - protein, 11 aa, synthetic construct
  REGION 1..11: note = Description of Artificial Sequence: Syntheticpeptide
QASQDISNYLH

SEQ ID NO: 522 - protein, 7 aa, synthetic construct
  REGION 1..7: note = Description of Artificial Sequence: Syntheticpeptide
DASDLET

SEQ ID NO: 523 - protein, 9 aa, synthetic construct
  REGION 1..9: note = Description of Artificial Sequence: Syntheticpeptide
QQYDHLPIT

SEQ ID NO: 524 - protein, 11 aa, synthetic construct
  REGION 1..11: note = Description of Artificial Sequence: Syntheticpeptide
QASQDISNYLN

SEQ ID NO: 525 - protein, 7 aa, synthetic construct
  REGION 1..7: note = Description of Artificial Sequence: Syntheticpeptide
DASNLET

SEQ ID NO: 526 - protein, 9 aa, synthetic construct
  REGION 1..9: note = Description of Artificial Sequence: Syntheticpeptide
QQYDNLPYT

SEQ ID NO: 527 - protein, 16 aa, synthetic construct
  REGION 1..16: note = Description of Artificial Sequence: Syntheticpeptide
RSSQSLLYSNGYNYLD

SEQ ID NO: 528 - protein, 7 aa, synthetic construct
  REGION 1..7: note = Description of Artificial Sequence: Syntheticpeptide
LNSNRAS

SEQ ID NO: 529 - protein, 9 aa, synthetic construct
  REGION 1..9: note = Description of Artificial Sequence: Syntheticpeptide
MQALQTPLT

SEQ ID NO: 530 - protein, 8 aa, synthetic construct
  REGION 1..8: note = Description of Artificial Sequence: Synthetic8xHis tag
HHHHHHHH

SEQ ID NO: 531 - protein, 6 aa, synthetic construct
  REGION 1..6: note = Description of Artificial Sequence: Synthetic6xHis tag
HHHHHH

(19) United States
(12) Patent Application Publication
(10) Pub. No.: US 2025/0011446 A1
(43) Pub. Date: Jan. 9, 2025

(54) ANTIBODIES SPECIFIC FOR CD70 AND THEIR USES

(71) Applicant: Pfizer Inc., New York, NY (US)

(72) Inventors: Siler PANOWSKI, Berkeley, CA (US); Tao SAI, Foster City, CA (US); Barbra Johnson SASU, San Francisco, CA (US); Surabhi SRIVATSA SRINIVASAN, San Francisco, CA (US); Thomas John VAN BLARCOM, Oakland, CA (US)

(21) Appl. No.: 18/639,749

(22) Filed: Apr. 18, 2024

Related U.S. Application Data
(62) Division of application No. 17/830,324, filed on Jun. 1, 2022, now Pat. No. 11,987,634.
Application No. 17/830,324 is a division of application No. 16/264,485, filed on Jan. 31, 2019, now Pat. No. 11,377,500.
(60) Provisional application No. 62/641,873, filed on Mar. 12, 2018.
(60) Provisional application No. 62/625,019, filed on Feb. 1, 2018.

(51) Int. Cl. C07K 16/28; A61K 39/00; A61P 35/00

(52) U.S. Cl. CPC C07K 16/2875; A61P 35/00; C07K 16/2809; A61K 2039/505; C07K 2317/31; C07K 2317/526; C07K 2317/53; C07K 2317/565; C07K 2317/732; C07K 2317/734; C07K 2317/76; C07K 2317/92

(57) ABSTRACT

The present invention provides antibodies that specifically bind to CD70 (Cluster of Differentiation 70). The invention further provides bispecific antibodies that bind to CD70 and another antigen (e.g., CD3). The invention further relates to antibody encoding nucleic acids, and methods of obtaining such antibodies (monospecific and bispecific). The invention further relates to therapeutic methods for use of these antibodies for the treatment of CD70-mediated pathologies, including cancer.

### CROSS-REFERENCE TO RELATED APPLICATIONS

This application is a divisional application of U.S. patent application Ser. No. 17/830,324, filed Jun. 1, 2022, issued May 21, 2024 as U.S. Pat. No. 11,987,634, which is a divisional application of U.S. patent application Ser. No. 16/264,485, filed Jan. 31, 2019, issued Jul. 5, 2022 as U.S. Pat. No. 11,377,500, which claims priority to U.S. Provisional Patent Appl. No. 62/641,873, filed Mar. 12, 2018, and U.S. Provisional Patent Appl. No. 62/625,019, filed Feb. 1, 2018, each of which is incorporated herein by reference in its entirety.

### REFERENCE TO SEQUENCE LISTING

This application is being filed electronically and includes an electronically submitted sequence listing in .xml format. The .xml file contains a sequence listing entitled “ALG015us3.xml” created on Apr. 9, 2024, and having a size of 691,055 bytes. The sequence listing contained in this xml file is part of the specification and is incorporated herein by reference in its entirety.

### FIELD

The present invention relates to antibodies, e.g., full length antibodies or antigen binding fragments thereof, that specifically bind to Cluster of Differentiation 70 (CD70). The invention further relates to heteromultimeric antibodies (e.g., bispecific antibodies) comprising CD70 antibody on one arm. Compositions comprising the CD70 antibodies, methods for producing and purifying such antibodies, and their use in diagnostics and therapeutics are also provided.

### BACKGROUND

Renal Cell Carcinoma (RCC) is a cancer that originates in the renal cortex and accounts for about 90% of cancers in the kidney. Based on histology, RCC can be classified into several sub-types, of which Clear Cell Renal Cell Carcinoma (ccRCC) is the most common and leads to the most deaths. Each year, over 320,000 cases of RCC are reported worldwide leading to roughly 140,000 deaths. The incidence of RCC has risen steadily over the last 10 years and accounts for 2-3% of all adult malignancies. Patients with early stage localized tumors can opt for surgical resection; however, localized disease can undergo early hematogenous dissemination leading to metastasis. Sites of early metastases include lungs, lymph nodes, liver, bone, and brain; less commonly the adrenal glands, and the contralateral kidney. Patients with advanced disease face high morbidity rates with a 5-year median survival rate of 53% for stage III disease and only 8% for metastatic disease. Current first-line treatment options for advanced disease include small molecule Tyrosine Kinase Inhibitors (TKIs) such as sunitinib and pazopanib that target Vascular Endothelial Growth Factor (VEGF) receptor, monoclonal antibody targeting VEGF such as bevacizumab, mammalian target of Rapamycin (mTOR) inhibitor temsirolimus, as well as high dose IL-2. Although these VEGF-targeted therapies have improved over-all survival, long-term drug resistance leads to disease relapse and treatment for advanced disease still remains an unmet need (see, e.g., Zarrabi, K. et al., Journal of Hematology and Oncology, 10:38 (2017)).

Cluster of Differentiation 70 (CD70, CD27LG or TNFSF7) is a member of the tumor necrosis factor (TNF) superfamily and the ligand for CD27, a TNF superfamily receptor. The transient interaction between CD27 and CD70 provides T cell costimulation complementary to that provided by CD28. CD70 is expressed on hematological cancers such as Non-Hodgkin's Lymphoma and Hodgkin's disease as well as on solid tumors such as Glioblastoma and Renal Cell Carcinoma; with its expression on ccRCC being nearly uniform (see e.g., Grewal I., et al., Expert Opinion on Therapeutic Targets, 12(3): 341-351 (2008)).

CD70 bispecific antibody in the form of T-cell engaging bispecific approach has been developed recently. However, a limitation of many bispecific formats is that they are of small molecular weight, and of short half-life, thus requiring continuous infusion. Accordingly, there remains a need for antibodies (e.g., monospecific or bispecific) treating cancer where CD70 is expressed and in particular mRCC with improved efficacy and safety profile, and suitable for use with human patients.

### SUMMARY

The invention disclosed herein is directed to antibodies (e.g., monospecific or bispecific antibodies) that specifically bind to Cluster of Differentiation 70 (CD70). In some embodiments, the CD70 antibodies as described herein in the full-length bispecific format have longer half-life, minimized Fc-interaction, and minimized non-specific cytokine release in vivo via interaction with immune cells.

Accordingly, in one aspect, the invention provides an isolated antibody which specifically binds to CD70, wherein the antibody comprises (a) a heavy chain variable (VH) region comprising (i) a VH complementarity determining region one (CDR1) comprising the sequence shown in SEQ ID NO: 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 332, 333, 334, 338, 339, 340, 344, 345, 346, 350, 351, 352, 356, 357, 358, 362, 363, 364, 368, 369, 370, 374, 375, 376, 380, 381, 382, 386, 387, 388, 392, 393, 394, 398, 399, 400, 404, 405, 406, 410, 411, 412, 416, 437, 418, 422, 423, 424, 428, 429, 430, 434, 435, 436, 440, 441, 442, 446, 447, 448, 452, 453, 454, 458, 459 or 460; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 335, 336, 341, 342, 347, 348, 353, 354, 359, 360, 365, 366, 371, 372, 377, 378, 383, 384, 389, 390, 395, 396, 401, 402, 407, 408, 413, 414, 419, 420, 425, 426, 431, 432, 437, 438, 443, 444, 449, 450, 455, 456, 461 or 462; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 337, 343, 349, 355, 361, 367, 373, 379, 385, 391, 397, 403, 409, 415, 421, 427, 433, 439, 445, 451, 457 or 463; and/or a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 464, 467, 470, 473, 476, 479, 482, 485, 488, 491, 494, 497, 500, 503, 506, 509, 512, 515, 518, 521, 524 or 527; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 465, 468, 471, 474, 477, 480, 483, 486, 489, 492, 495, 498, 501, 504, 507, 510, 513, 516, 519, 522, 525 or 528; and (iii) a VL CDR3 comprising the sequence shown in SEQ ID NO: 195, 198, 201, 204, 207, 210, 213, 216, 219, 222, 225, 228, 231, 234, 237, 240, 243, 246, 249, 252, 255, 258, 261, 264, 466, 469, 472, 475, 478, 481, 484, 487, 490, 493, 496, 499, 502, 505, 508, 511, 514, 517, 520, 523, 526 or 529.

In another aspect, provided is an isolated antibody which specifically binds to CD70, wherein the antibody comprises: a VH region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 289, 291, 293, 295, 297, 299, 301, 303, 305, 307, 309, 311, 313, 315, 317, 319, 321, 323, 325, 327, 329 or 331; and/or a VL region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 288, 290, 292, 294, 296, 298, 300, 302, 304, 306, 308, 310, 312, 314, 316, 318, 320, 322, 324, 326, 328 or 330. In some embodiments, the VH region as described herein comprises a variant with one or several conservative amino acid substitutions in residues that are not within a CDR and/or the VL region as described herein comprises a variant with one or several amino acid substitutions in amino acids that are not within a CDR. For example, in some embodiments, the VH or VL region can comprise an amino acid sequence described above or a variant thereof with no more than 10, 9, 8, 7, 6, 5, 4, 3, 2, or 1 conservative substitutions in residues that are not within a CDR.

In some embodiments, provided is an isolated antibody which specifically binds to CD70, wherein the antibody comprises: a VH region comprising the sequence shown in SEQ ID NO: 18; and/or a VL region comprising the sequence shown in SEQ ID NO: 17.

In some embodiments, provided is an antibody which specifically binds to CD70 and competes with an isolated antibody provided herein which specifically binds to CD70.

In another aspect, provided is a bispecific antibody wherein the bispecific antibody is a full-length antibody, comprising a first antibody variable domain of the bispecific antibody specifically binding to a target antigen (e.g., CD70), and comprising a second antibody variable domain of the bispecific antibody capable of recruiting the activity of a human immune effector cell by specifically binding to an effector antigen (e.g., Cluster of differentiation 3 (CD3)) located on the human immune effector cell. In some embodiments, the first antibody variable domain comprises a heavy chain variable (VH) region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 289, 291, 293, 295, 297, 299, 301, 303, 305, 307, 309, 311, 313, 315, 317, 319, 321, 323, 325, 327, 329 or 331; and/or a light chain variable (VL) region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 288, 290, 292, 294, 296, 298, 300, 302, 304, 306, 308, 310, 312, 314, 316, 318, 320, 322, 324, 326, 328 or 330. In some embodiments, the first antibody variable domain comprises (a) a heavy chain variable (VH) region comprising (i) a VH complementarity determining region one (CDR1) comprising the sequence shown in SEQ ID NO: 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 332, 333, 334, 338, 339, 340, 344, 345, 346, 350, 351, 352, 356, 357, 358, 362, 363, 364, 368, 369, 370, 374, 375, 376, 380, 381, 382, 386, 387, 388, 392, 393, 394, 398, 399, 400, 404, 405, 406, 410, 411, 412, 416, 437, 418, 422, 423, 424, 428, 429, 430, 434, 435, 436, 440, 441, 442, 446, 447, 448, 452, 453, 454, 458, 459 or 460; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 335, 336, 341, 342, 347, 348, 353, 354, 359, 360, 365, 366, 371, 372, 377, 378, 383, 384, 389, 390, 395, 396, 401, 402, 407, 408, 413, 414, 419, 420, 425, 426, 431, 432, 437, 438, 443, 444, 449, 450, 455, 456, 461 or 462; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 337, 343, 349, 355, 361, 367, 373, 379, 385, 391, 397, 403, 409, 415, 421, 427, 433, 439, 445, 451, 457 or 463; and/or (b) a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 464, 467, 470, 473, 476, 479, 482, 485, 488, 491, 494, 497, 500, 503, 506, 509, 512, 515, 518, 521, 524 or 527; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 465, 468, 471, 474, 477, 480, 483, 486, 489, 492, 495, 498, 501, 504, 507, 510, 513, 516, 519, 522, 525 or 528; and (iii) a VL CDR3 comprising the sequence shown in SEQ ID NO: 195, 198, 201, 204, 207, 210, 213, 216, 219, 222, 225, 228, 231, 234, 237, 240, 243, 246, 249, 252, 255, 258, 261, 264, 466, 469, 472, 475, 478, 481, 484, 487, 490, 493, 496, 499, 502, 505, 508, 511, 514, 517, 520, 523, 526 or 529.

In some embodiments, the second antibody variable domain comprises the VH and/or VL region specific against CD3. For example, the second antibody variable domain comprises a heavy chain variable (VH) region comprising a VH CDR1, VH CDR2, and VH CDR3 of the VH sequence shown in SEQ ID NO:266; and/or a light chain variable (VL) region comprising a VL CDR1, VL CDR2, and VL CDR3 of the VL sequence shown in SEQ ID NO: 265. In some embodiments, the second antibody variable domain comprises (a) a VH region comprising (i) a VH CDR1 comprising the sequence shown in SEQ ID NO: 267, 268, or 269; (ii) a VH CDR2 comprising the sequence shown in SEQ ID NO: 270 or 271; and iii) a VH CDR3 comprising the sequence shown in SEQ ID NO: 272; and/or a VL region comprising (i) a VL CDR1 comprising the sequence shown in SEQ ID NO: 273; (ii) a VL CDR2 comprising the sequence shown in SEQ ID NO: 274; and (iii) a VL CDR3 comprising the sequence shown in SEQ ID NO: 275.

In some embodiments, the antibodies described herein comprise a constant region. In some embodiments, the antibodies described herein are of the human IgG1, IgG2 or IgG2Aa, IgG3, or IgG4 subclass. In some embodiments, the antibodies described herein comprise a glycosylated constant region. In some embodiments, the antibodies described herein comprise a constant region having decreased binding affinity to one or more human Fc gamma receptor(s).

In some embodiments, both the first and the second antibody variable domains of the bispecific antibody comprise amino acid modifications at positions 223, 225, and 228 (e.g., (C223E or C223R), (E225R), and (P228E or P228R)) in the hinge region and at position 409 or 368 (e.g., K409R or L368E (EU numbering scheme)) in the CH3 region of human IgG2 (SEQ ID NO: 279).

In some embodiments, both the first and the second antibody variable domains of the bispecific antibody comprise amino acid modifications at position 265 (e.g., D265A) of the human IgG2.

In some embodiments, both the first and the second antibody variable domains of the bispecific antibody comprise amino acid modifications at one or more of positions 265 (e.g., D265A), 330 (e.g., A330S), and 331 (e.g., P331S) of the human IgG2. In some embodiments, both the first and the second antibody variable domains of the bispecific antibody comprise amino acid modifications at each of positions 265 (e.g., D265A), 330 (e.g., A330S), and 331 (e.g., P331S) of the human IgG2.

In other embodiments, the invention provides pharmaceutical compositions comprising any of the antibodies described herein.

The invention also provides cell lines that recombinantly produce any of the antibodies described herein.

The invention also provides nucleic acids encoding any of the antibodies described herein. The invention also provides nucleic acids encoding a heavy chain variable region and/or a light chain variable region of any of the antibodies described herein.

The invention also provides a host cell comprising a nucleic acid or vector provided herein. Also provided is a method of producing an antibody (e.g. monospecific or bispecific) provided herein, comprising culturing a host cell provided herein under conditions that result in production of the antibody, and isolating the antibody from the host cell or culture.

The invention also provides kits comprising an effective amount of any of the antibodies or antibody conjugates described herein.

Also provided is an antibody or bispecific antibody provided herein for use as a medicament.

The invention also provides methods of treating subjects in need thereof comprising providing the isolated antibodies or bispecific antibodies described herein, and administering said antibodies to said subject.

Also provided are methods of treating a condition associated with malignant cells expressing CD70 in a subject comprising administering to a subject in need thereof an effective amount of a pharmaceutical composition comprising the antibodies as described herein. In some embodiments, the condition is a cancer. In some embodiments, the cancer is an CD70 related cancer (e.g., any cancer with CD70 expression) selected from the group consisting of Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer.

In another aspect, the invention provides a method of inhibiting tumor growth or progression in a subject who has malignant cells expressing CD70, comprising administering to the subject in need thereof an effective amount of a pharmaceutical composition comprising the isolated antibodies or bispecific antibodies, as described herein.

In another aspect, the invention provides a method of inhibiting metastasis of malignant cells expressing CD70 in a subject, comprising administering to the subject in need thereof an effective amount of the pharmaceutical composition comprising the isolated antibodies or bispecific antibodies, as described herein.

In another aspect, the invention provides a method inducing tumor regression in a subject who has malignant cells expressing CD70, comprising administering to the subject in need thereof an effective amount of the pharmaceutical composition of a pharmaceutical composition comprising the isolated antibodies or bispecific antibodies, as described herein.

### DETAILED DESCRIPTION

The invention disclosed herein provides antibodies (e.g., monospecific or bispecific) that specifically bind to CD70 (e.g., human CD70). The invention also provides polynucleotides encoding these antibodies, compositions comprising these antibodies, and methods of making and using these antibodies. The invention also provides methods for treating a condition associated with CD70-mediated pathologies in a subject, such as cancer. In particular, the inventors of the present invention have discovered that the CD70 antibodies as described herein in the full-length bispecific format have longer half-life, minimized Fc-interaction, and minimized non-specific cytokine release in vivo via interaction with the immune cells.

### General Techniques

The practice of the present invention will employ, unless otherwise indicated, conventional techniques of molecular biology (including recombinant techniques), microbiology, cell biology, biochemistry, immunology, virology, monoclonal antibody generation and engineering, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M.J. Gait, ed., 1984); Methods in Molecular Biology, Humana Press; Cell Biology: A Laboratory Notebook (J.E. Cellis, ed., 1998) Academic Press; Animal Cell Culture (R.I. Freshney, ed., 1987); Introduction to Cell and Tissue Culture (J.P. Mather and P.E. Roberts, 1998) Plenum Press; Cell and Tissue Culture: Laboratory Procedures (A. Doyle, J.B. Griffiths, and D.G. Newell, eds., 1993-1998) J. Wiley and Sons; Methods in Enzymology (Academic Press, Inc.); Handbook of Experimental Immunology (D.M. Weir and C.C. Blackwell, eds.); Gene Transfer Vectors for Mammalian Cells (J.M. Miller and M.P. Calos, eds., 1987); Current Protocols in Molecular Biology (F.M. Ausubel et al., eds., 1987); PCR: The Polymerase Chain Reaction, (Mullis et al., eds., 1994); Current Protocols in Immunology (J.E. Coligan et al., eds., 1991); Short Protocols in Molecular Biology (Wiley and Sons, 1999); Immunobiology (C.A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J.D. Capra, eds., Harwood Academic Publishers, 1995).

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab∝, F(ab′)₂, Fv), single chain (ScFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., CD70). Antigen binding functions of an antibody can be performed by fragments of an intact antibody. Examples of binding fragments encompassed within the term “antigen binding fragment” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody or a polypeptide that “preferentially binds” or “specifically binds” (used interchangeably herein) to a target (e.g., CD70 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an CD70 epitope is an antibody that binds this epitope with greater affinity, avidity, more readily, and/or with greater duration than it binds to other CD70 epitopes or non-CD70 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1): 86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The terms “polypeptide”, “oligopeptide”, “peptide” and “protein” are used interchangeably herein to refer to chains of amino acids of any length. For example, the chain may be relatively short (e.g., 10-100 amino acids), or longer. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “monovalent antibody” comprises one antigen binding site per molecule (e.g., IgG or Fab). In some instances, a monovalent antibody can have more than one antigen binding sites, but the binding sites are from different antigens.

A “monospecific antibody” comprises two identical antigen binding sites per molecule (e.g. IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g. Fab)

A “bivalent antibody” comprises two antigen binding sites per molecule (e.g., IgG). In some instances, the two binding sites have the same antigen specificities. However, bivalent antibodies may be bispecific.

A “bispecific” or “dual-specific” is a hybrid antibody having two different antigen binding sites. The two antigen binding sites of a bispecific antibody bind to two different epitopes, which may reside on the same or different protein targets.

A “bifunctional” is antibody is an antibody having identical antigen binding sites (i.e., identical amino acid sequences) in the two arms but each binding site can recognize two different antigens.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

A “heterodimer,” “heterodimeric protein,” “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

The “hinge region,” “hinge sequence”, and variations thereof, as used herein, includes the meaning known in the art, which is illustrated in, for example, Janeway et al., ImmunoBiology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The “immunoglobulin-like hinge region,” “immunoglobulin-like hinge sequence,” and variations thereof, as used herein, refer to the hinge region and hinge sequence of an immunoglobulin-like or an antibody-like molecule (e.g., immunoadhesins). In some embodiments, the immunoglobulin-like hinge region can be from or derived from any IgG1, IgG2, IgG3, or IgG4 subtype, or from IgA, IgE, IgD or IgM, including chimeric forms thereof, e.g., a chimeric IgG1/2 hinge region.

The term “immune effector cell” or “effector cell as used herein refers to a cell within the natural repertoire of cells in the human immune system which can be activated to affect the viability of a target cell. The viability of a target cell can include cell survival, proliferation, and/or ability to interact with other cells.

Antibodies of the invention can be produced using techniques well known in the art, e.g., recombinant technologies, phage display technologies, synthetic technologies or combinations of such technologies or other technologies readily known in the art (see, for example, Jayasena, S.D., Clin. Chem., 45:1628-50, 1999 and Fellouse, F.A., et al, J. Mol. Biol., 373 (4): 924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As known in the art, a “constant region” of an antibody refers to the constant region of the antibody light chain or the constant region of the antibody heavy chain, either alone or in combination.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant regions, CH2 and CH3.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, Ann. Rev. Immunol., 9:457-92, 1991; Capel et al., Immunomethods, 4:25-34, 1994; and de Haas et al., J. Lab. Clin. Med., 126:330-41, 1995. “FcR” also includes the neonatal receptor, FcRn, which is responsible for the transfer of maternal IgGs to the fetus (Guyer et al., J. Immunol., 117:587, 1976; and Kim et al., J. Immunol., 24:249, 1994).

The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, e.g. from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

The term “effector function” refers to the biological activities attributable to the Fc region of an antibody. Examples of antibody effector functions include, but are not limited to, antibody-dependent cell-mediated cytotoxicity (ADCC), Fc receptor binding, complement dependent cytotoxicity (CDC), phagocytosis, C1q binding, and down regulation of cell surface receptors (e.g., B cell receptor; BCR). See, e.g., U.S. Pat No. 6,737,056. Such effector functions generally require the Fc region to be combined with a binding domain (e.g., an antibody variable domain) and can be assessed using various assays known in the art for evaluating such antibody effector functions. An exemplary measurement of effector function is through Fcγ3 and/or C1q binding.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” or “CDC” refers to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of CD70 expressing tumor, remission of an CD70 associated disease (e.g., cancer), decreasing symptoms resulting from an CD70 associated disease (e.g., cancer), increasing the quality of life of those suffering from an CD70 associated disease (e.g., cancer), decreasing the dose of other medications required to treat an CD70 associated disease (e.g., cancer), delaying the progression of an CD70 associated disease (e.g., cancer), curing an CD70 associated disease (e.g., cancer), and/or prolong survival of patients having an CD70 associated disease (e.g., cancer).

“Ameliorating” means a lessening or improvement of one or more symptoms as compared to not administering an CD70 antibody (monospecific or bispecific). “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various CD70 associated diseases or conditions (such as for example multiple myeloma), decreasing the dose of other medications required to treat the disease, enhancing the effect of another medication, and/or delaying the progression of the CD70 associated disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, PA, 1990; and Remington, The Science and Practice of Pharmacy 21st Ed. Mack Publishing, 2005).

The term “acyl donor glutamine-containing tag” or “glutamine tag” as used herein refers to a polypeptide or a protein containing one or more Gln residue(s) that acts as a transglutaminase amine acceptor. See, e.g., WO2012059882 and WO2015015448.

The term “kₒₙ” or “kₐ”, as used herein, refers to the rate constant for association of an antibody to an antigen. Specifically, the rate constants (kₒₙ/kₐ and k_{off}/k_{d}) and equilibrium dissociation constants are measured using whole antibody (i.e. bivalent) and monomeric CD70 proteins (e.g., Histidine-tagged CD70 fusion protein).

The term “k_{off}” or “k_{d}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 per cent of the indicated value, whichever is greater.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the present invention. The materials, methods, and examples are illustrative only and not intended to be limiting.

### CD70 Antibodies and Methods of Making Thereof

The present invention provides an antibody that binds to CD70 [e.g., human CD70 (e.g., accession number: NP_004110 or SEQ ID NO: 235)] and characterized by any one or more of the following characteristics: (a) treat, prevent, ameliorate one or more symptoms of a condition associated with malignant cells expressing CD70 in a subject (e.g., cancer such as AML); (b) inhibit tumor growth or progression in a subject (who has a malignant tumor expressing CD70); (c) inhibit metastasis of cancer (malignant) cells expressing CD70 in a subject (who has one or more malignant cells expressing CD70); (d) induce regression (e.g., long-term regression) of a tumor expressing CD70; (e) exert cytotoxic activity in malignant cells expressing CD70; (f) block CD70 interaction with other yet to be identified factors; and/or (g) induce bystander effect that kill or inhibit growth of non-CD70 expressing malignant cells in the vicinity.

In one aspect, provided is an isolated antibody which specifically binds to CD70, wherein the antibody comprises (a) a heavy chain variable (VH) region comprising (i) a VH complementarity determining region one (CDR1) comprising the sequence shown in 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 332, 333, 334, 338, 339, 340, 344, 345, 346, 350, 351, 352, 356, 357, 358, 362, 363, 364, 368, 369, 370, 374, 375, 376, 380, 381, 382, 386, 387, 388, 392, 393, 394, 398, 399, 400, 404, 405, 406, 410, 411, 412, 416, 437, 418, 422, 423, 424, 428, 429, 430, 434, 435, 436, 440, 441, 442, 446, 447, 448, 452, 453, 454, 458, 459 or 460; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 335, 336, 341, 342, 347, 348, 353, 354, 359, 360, 365, 366, 371, 372, 377, 378, 383, 384, 389, 390, 395, 396, 401, 402, 407, 408, 413, 414, 419, 420, 425, 426, 431, 432, 437, 438, 443, 444, 449, 450, 455, 456, 461 or 462; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 337, 343, 349, 355, 361, 367, 373, 379, 385, 391, 397, 403, 409, 415, 421, 427, 433, 439, 445, 451, 457 or 463; and/or a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 464, 467, 470, 473, 476, 479, 482, 485, 488, 491, 494, 497, 500, 503, 506, 509, 512, 515, 518, 521, 524 or 527; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 465, 468, 471, 474, 477, 480, 483, 486, 489, 492, 495, 498, 501, 504, 507, 510, 513, 516, 519, 522, 525 or 528; and (iii) a VL CDR3 comprising the sequence shown in SEQ ID NO: 195, 198, 201, 204, 207, 210, 213, 216, 219, 222, 225, 228, 231, 234, 237, 240, 243, 246, 249, 252, 255, 258, 261, 264, 466, 469, 472, 475, 478, 481, 484, 487, 490, 493, 496, 499, 502, 505, 508, 511, 514, 517, 520, 523, 526 or 529.

In another aspect, provided is an isolated antibody which specifically binds to CD70, wherein the antibody comprises: a VH region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 289, 291, 293, 295, 297, 299, 301, 303, 305, 307, 309, 311, 313, 315, 317, 319, 321, 323, 325, 327, 329 or 331; and/or a VL region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 288, 290, 292, 294, 296, 298, 300, 302, 304, 306, 308, 310, 312, 314, 316, 318, 320, 322, 324, 326, 328 or 330.

In some embodiments, provided is an antibody having any one of partial light chain sequence as listed in Table 1 and/or any one of partial heavy chain sequence as listed in Table 1. In Table 1, the underlined sequences are CDR sequences according to Kabat and in bold according to Chothia.

TABLE 1

| mAb | Light Chain | Heavy Chain |
|---|---|---|
| 31H1 | DIVMTQNPLSSPVTLGQPASISCRS | QVQLVQSGAEVKKPGSSVKVSCKA |
| | SQSLVHSDGNTYLSWLQQRPGQS | SGGTFSSYGFSWRQAPGQGLEW |
| | PRLLIYKISNRFSGVPDRFSGSGAG | MGGIIPIFGSANYAQKFQGRVTITAD |
| | TDFTLKISRVEAEDVGVYYCMQAT | KSTSTVYMELISLRSEDTAVYYCAR |
| | QFPLTIGGGSKVEIK | GGSSSPFAYWGQGTLVTVSS |
| | (SEQ ID NO: 1) | (SEQ ID NO: 2) |
| 63B2 | DIVMTQTPLSSPVTLGQPASISCRS | QVQLVQSGAEVKKPGSSVKVSCKA |
| | SQSLVHSDGNTYLSWLQQRPGQS | SGGTFSSYGFSWRQAPGQGLEW |
| | PRLLIYKISNRFSGVPDRFSGSGAG | MGGIIPIFGTANYAQKFQGRVTITAD |
| | TDFTLKISRVEAEDVGVYYCMQAT | KSTSTVFMELISLRSEYTAVYYCAR |
| | QFPLTIGGGSKVEIK | GGSSSPFAYWGQGTLVTVSS |
| | (SEQ ID NO: 3) | (SEQ ID NO: 4) |
| 40E3 | DIQMTQSPSSLSASVGDRVTITCRA | QVQLQESGPGLVKPSETLSLTCTVS |
| | SQGISNYLAWFQQKPGKAPKSLIY | GGSISSYYWNWIRQPPGKGLEWIG |
| | AASSLQSGVPSKFSGSGSGTDFTL | YIYYSGSTNYNPSLKSRVTISVDTSK |
| | TISSLQPEDFATYYCQQYNSYPLTF | NQFSLKLRSVTAADTAVYYCARDIR |
| | GGGTKVEIK | TWGQGTLVTVSS |
| | (SEQ ID NO: 5) | (SEQ ID NO: 6) |
| 42C3 | DVVMTQSPLSLPVTLGQPASISCRS | EVQLVESGGGLVQPGGSLRLSCAA |
| | SQSLVYSDENTYLNWFQQRPGQS | SGFTFRNSWMSWVRQAPGKGLEW |
| | LRRLIYQVSNRDSGVPDRFSGSGS | VANIKRDGSEKYYVDSVKGRFTISR |
| | GTDFTLKISRVEAEDVGVYFCMQG | DNAKNSLYLQMNSLRAEDTAVYYC |
| | TYWPPTFGGGTKVEIK | ARDQTGSFDYWGQGTLVTVSS |
| | (SEQ ID NO: 7) | (SEQ ID NO: 8) |
| 45F11 | EIVMTQSPATLSMSLGERATLSCRA | QVQLRGSGPGLVKPSETLSLTCTVS |
| | SQSVSSSLAWYQQKPGQAPRLLIY | DDSISVYYWSWIRQPAGKGLEWIGR |
| | GASTRATGIPARFGGSGSGTEFTL | VYSSGNINYNPSLESRVTMSVDTSK |
| | TISSLQSEDFAVYYCQQYINWPHFG | SRFSLNLSSVTAADTAVYYCARGLD |
| | GGTKVEIK | AFDIWGQGTMVTVSS |
| | (SEQ ID NO: 9) | (SEQ ID NO: 10) |
| 64F9 | DIQMTQSPSSLSASVGDRVTITCQA | EVQLLESGGGLVQPGESLRLSCEVS |
| | SQDISNYLNWYQQKPGKAPKILIYG | GFTFTSYAMSWVRQVPGKGLEWVS |
| | ASNLETGVPSRFSGSGSGTDFTFAI | IISGVAFTTYYADSVKGRFTISRDHS |
| | SSLQPEDVATYYCQQYDNFPITFG | KNTLYLQMNGLRAEDTAVYYCVKV |
| | QGTRLEIK | DGEVYWGQGTLVTVSS |
| | (SEQ ID NO: 11) | (SEQ ID NO: 12) |
| 72C2 | EIVMTQSPDTLSVSPGERAILSCRA | QVQLVQSGAEVKKPGSSVKVSCEA |
| | SQSVSSNLAWYQQKPGQAPRLLIY | SGGTFITYAISWVRQAPGQGLEWM |
| | SASTRASGIPARFSGSGSGTEFTLS | GGIIPFFGTANYAQKFQGRVTITADK |
| | ISSLQSEDFAVYYCQQYDNWPPLT | STSTASMELRSLRSEDTAMYYCAQ |
| | FGGGTKVEIK | WELFFFDFWGQGTPVTVSS |
| | (SEQ ID NO: 13) | (SEQ ID NO: 14) |
| 2F10 | EIVLTQSPGTLSLSPGERATLSCRA | AVQLVESGGGLVQPGGSLRLSCAA |
| | SQSVSSSYLAWYQQQPGQAPRLLI | SGFTFTYYSMNWWRQAPGKGLEW |
| | YGASSRATGIPDRFSGSGSGTDFT | VSHISIRSSTIYFADSAKGRFTISRDN |
| | LTISRLEPEDFAIYYCQQYGSSPLTF | AKNSLYLQMNSLRDEDTAVYYCAR |
| | GGGTKVEIK | GSGWYGDYFDYWGQGTLVTVSS |
| | (SEQ ID NO: 15) | (SEQ ID NO: 16) |
| 4F11 | DIQMTQSPSAMSASVGDRVTITCR | QVTLKESGPVLVKPTETLTLTCTVS |
| | ASQDISNYLAWFQQKPGKVPKRLI | GFSLSNARMGVTWIRQPPGKALEW |
| | YAASSLQSGVPSRFSGSGSGTEFT | LAHIFSNDEKSYSTSLKSRLTISKDT |
| | LTISSLLPEDFATYYCLQLNSFPFTF | SKTQWLTMTNMDPVDTATYYCARI |
| | GGGTKVEIN | RDYYDISSYYDYWGQGTLVSVSS |
| | (SEQ ID NO: 17) | (SEQ ID NO: 18) |
| 10H10 | DIQMTQSPSSVSASVGDRVTITCRA | EVQLVESGGGLVQPGGSLRLSCAV |
| | SQGISSWLAWYQQKPGKAPKVLIY | SGFTFSNHNIHWVRQAPGKGLEWIS |
| | AASSLQSGVPSRFSGSGSGTDFTL | YISRSSSTIYYADSVKGRFTISRDNA |
| | TISSLQPEDFATYYCQQAFSFPFTF | KNSLYLQMNSLRDEDTAVYYCARD |
| | GPGTKVDIK | HAQWYGMDVWGQGTTVTVSS |
| | (SEQ ID NO: 19) | (SEQ ID NO: 20) |
| 17G6 | DIVMTQSPDSLAVSLGERATINCKS | EVQLVESGGGLVQPGGSLRLSCVA |
| | SQSVLYSYNNKNYVAWYQQKPGQ | SGFTFSSYWMSWVRQAPGKGLEW |
| | PPNLLIFWASTRESGVPDRFSGSG | VASIKQDGSEKYYVDSVKGRFTISR |
| | SGTDFTLTISSLQAEDVAVYYCQQY | DNAKNSVYLQMNSLRAEDTGVYYC |
| | YSTLTFGGGTKVEIK | AREGVNWGWRLYWHFDLWGRGTL |
| | (SEQ ID NO: 21) | VTVSS |
| | | (SEQ ID NO: 22) |
| 65E11 | EIVLTQSPGTLSLSPGERVTLSCRA | EVQWESGGGLVQPGGSLRLSCAA |
| | SQSVSSSYLAWYQQKPGQAPRLLI | SGFTFSSYSMNWVRQAPGKGLEW |
| | YDASSRATGIPDRFSGSGSGTDFT | VSHSSISRGNIYFADSVKGRFTISRD |
| | LTISRLEPEDFAVYYCQQYGSSPLT | NAKNSLYLQMNSLRDEDTAVYYCA |
| | FGGGTKVEIK | RGSGWYGDYFDYWGQGTLVTVSS |
| | (SEQ ID NO: 23) | (SEQ ID NO: 24) |
| P02B10 | ELQSVLTQPPSASGTPGQRVTISCS | EVQLLESGGGLVQPGGSLRLSCAA |
| | GSSSNIGSNYVYWYQQLPGTAPKL | SGFAFSNYAMSWVRQAPGKGLEW |
| | LIYRNNQRPSGVPDRFSGSKSGTS | VSAIRGGGGSTYYADSVKGRFTISR |
| | ASLAISGLRSEDEADYYCAAWDDS | DNSKNTLYLQMNSLRAEDTAVYYCA |
| | LSGVVFGGGTKLTVL | RDFISGTWYPDYWGQGTLVTVSS |
| | (SEQ ID NO: 25) | (SEQ ID NO: 26) |
| P07D03 | ELQSVLTQPPSASGTPGQRVTISCS | EVQLVQSGAEVKKPGESLKISCKGS |
| | GSRSNIGSNYVYWYQQLPGTAPKL | GYRFTSYWIGWVRQMPGKGLEWM |
| | LIYRNNQRPSGVPDRFSGSKSGTS | GSIYPDDSDTRYSPSFQGQVTISAD |
| | ASLAISGLRSEDEADYYCASWDGS | KSISTAYLQWSSLKASDTAMYYCAS |
| | LSAVVFGTGTKLTVL | STVDYPGYSYFDYWGQGTLVTVSS |
| | (SEQ ID NO: 27) | (SEQ ID NO: 28) |
| P08A02 | ELQSVLTQPPSASGTPGQRVTISCS | EVQLVQSGAEVKKPGESLKISCKGS |
| | GSSSNIGSNYVYWYQQLPGTAPKL | GYTFTNYWIAWWRQMPGKGLEWM |
| | LIYRNNQRPSGVPDRFSGSKSGTS | GIIYPDGSDTRYSPSFQGQVTISADK |
| | ASLAISGLRSEDEADYYCATWDDS | SISTAYLQWSSLKASDTAMYYCARD |
| | LGSPVFGTGTKLTVL | ITSWYYGEPAFDIWGQGTLVTVSS |
| | (SEQ ID NO: 29) | (SEQ ID NO: 30) |
| P08E02 | ELDIQMTQSPSSLSASVGDRVTITC | EVQLVQSGAEVKKPGESLKISCKGS |
| | RASQSISRYLNWYQQKPGKAPKLLI | GYSFTSSWIGWVRQMPGKGLEWM |
| | YAASILQTGVPSRFSGSGSGTDFT | GIIYPGDSDTRYSPSFQGQVTISADK |
| | LTISSLQPEDFATYYCQQSYSTTM | SISTAYLQWSSLKASDTAMYYCAKG |
| | WTFGQGTKVEIK | LSQAMTGFGFDYWGQGTLVTVSS |
| | (SEQ ID NO: 31) | (SEQ ID NO: 32) |
| P08F08 | ELQSVLTQPPSASGTPGQRVTISCS | EVQLVQSGAEVKKPGESLKISCKGS |
| | GSSSNIGSNYVNWYQQLPGTAPKL | GYGFTSYWIGWVRQMPGKGLEWM |
| | LIYGDYQRPSGVPDRFSGSKSGTS | GIIHPDDSDTKYSPSFQGQVTISADK |
| | ASLAISGLRSEDEADYYCATRDDSL | SISTAYLQWSSLKASDTAMYYCASS |
| | SGSVVFGTGTKLTVL | YLRGLWGGYFDYWGQGTLVTVSS |
| | (SEQ ID NO: 33) | (SEQ ID NO: 34) |
| P08G02 | ELDIQMTQSPSSLSASVGDRVTITC | EVQLVQSGAEVKKPGESLKISCKGS |
| | RASQSIYDYLHWYQQKPGKAPKLLI | GYTFPSSWIGWVRQMPGKGLEWM |
| | YDASNLQSGVPSRFSGSGSGTDFT | GIIYPDTSHTRYSPSFQGQVTISADK |
| | LTISSLQPEDFATYYCQQSYTTPLF | SISTAYLQWSSLKASDTAMYYCARA |
| | TFGQGTKVEIK | SYFDRGTGYSSWWMDVWGQGTLV |
| | (SEQ ID NO: 35) | TVSS |
| | | (SEQ ID NO: 36) |
| P12B09 | ELDIQMTQSPSSLSASVGDRVTITC | EVQLLESGGGLVQPGGSLRLSCAA |
| | RASQYIGRYLNWYQQKRGKAPKLL | SGFTFSQYSMSWVRQAPGKGLEW |
| | IHGATSLASGVPSRFSGSGSGTDF | VSAISGGGVSTYYADSVKGRFTISR |
| | TLTISSLQPEDFATYYCQQSYSTTS | DNSKNTLYLQMNSLRAEDTAVYYCA |
| | PTFGQGTKVEIK | SDISDSGGSHWYFDYWGQGTLVTV |
| | (SEQ ID NO: 37) | SS |
| | | (SEQ ID NO: 38) |
| P12F02 | ELQSVLTQPPSASGTPGQRVTISCS | EVQLLESGGGLVQPGGSLRLSCAA |
| | GSTSNIGRNYVYWYQQLPGTAPKL | SGFTFSSYAMSWVRQAPGKGLEW |
| | LIYRTNQRPSGVPDRFSGSKSGTS | VSTISGTGGTTYYADSVKGRFTISRD |
| | ASLAISGLRSEDEADYYCAAWDDS | NSKNTLYLQMNSLRAEDTAVYYCAK |
| | LSGRVFGTGTKLTVL | VRAGIDPTASDVWGQGTLVTVSS |
| | (SEQ ID NO: 39) | (SEQ ID NO: 40) |
| P12G07 | ELQSVLTQPPSASGTPGQRVTISCS | EVQLLESGGGLVQPGGSLRLSCAA |
| | GSSSNIGSNYVYWYQQLPGTAPKP | SGFTFNNFAMSWVRQAPGKGLEW |
| | LIYMNNQRPSGVPDRFSGSKSGTS | VSGISGSGDNTYYADSVKGRFTISR |
| | ASLAISGLRSEDEADYYCAAWDDS | DNSKNTLYLQMNSLRAEDTAVYYCA |
| | LSAVVFGTGTKLTVL | KDRDIGLGWYSYYLDVWGQGTLVT |
| | (SEQ ID NO: 41) | VSS |
| | | (SEQ ID NO: 42) |
| P13F04 | ELQSVLTQPPSASGTPGQRVTISCS | QVQLVQSGAEVKKPGSSVKVSCKA |
| | GSNSNIGTNYVSWYQQLPGTAPKL | SGGTFSSYAISWVRQAPGQGLEWM |
| | LIYRSSRRPSGVPDRFSGSKSGTS | GEIIPIFGTASYAQKFQGRVTITADES |
| | ASLAISGLRSEDEADYYCAAWDGS | TSTAYMELSSLRSEDTAVYYCARAG |
| | LSGHWVFGTGTKLTVL | WDDSWFDYWGQGTLVTVSS |
| | (SEQ ID NO: 43) | (SEQ ID NO: 44) |
| P15D02 | ELDIQMTQSPSSLSASVGDRVTITC | EVQLVQSGAEVKKPGESLKISCKGS |
| | RASQSIDTYLNWYQQKPGKAPKLLI | GYSFASYWIGWVRQMPGKGLEWM |
| | YSASSLHSGVPSRFSGSGSGTDFT | GVIYPGTSETRYSPSFQGQVTISAD |
| | LTISSLQPEDFATYYCQQSYSTTAW | KSISTAYLQWSSLKASDTAMYYCAK |
| | TFGQGTKVEIK | GLSASASGYSFQYWGQGTLVTVSS |
| | (SEQ ID NO: 45) | (SEQ ID NO: 46) |
| P16C05 | ELDIQMTQSPSSLSASVGDRVTITC | EVQLVQSGAEVKKPGESLKISCKGS |
| | RASQSIGQSLNWYQQKPGKAPKLL | GYSFTDYWIGWVRQMPGKGLEWM |
| | IYGASSLQSGVPSRFSGSGSGTDF | GMISPGGSTTIYRPSFQGQVTISADK |
| | TLTISSLQPEDFATYYCQQSYSTPIT | SISTAYLQWSSLKASDTAMYYCARE |
| | FGQGTKVEIK | MYTGGYGGSWYFDYWGQGTLVTV |
| | (SEQ ID NO: 47) | SS(SEQ ID NO: 48) |
| 10A1 | DIQMTQSPSTLSASVGDRVTITCRA | QVQLQESGPGLVKPSETLSLTCTVS |
| | SQSISTWLAWYQQKPGKAPKVLIY | GGSISYYYWTWIRQPPGKGLEWIG |
| | KASSLESGVPSRFSGSGSGTEFILT | HIYYSGSTNYNPSLKSRVTISIDTSK |
| | INSLQPDDFASYYCQQYKSYSHTF | NLFSLKLSSVTAADTAVYYCARAEG |
| | GQGTKLEIK | SIDAFDFWGQGTMVTVSS |
| | (SEQ ID NO: 288) | (SEQ ID NO: 289) |
| 10E2 | DIQMTQSPSTLSASVGDRVTITCRA | EVQLVESGGGLIQPGGSLRLSCAAS |
| | SQSISSWLAWYQQKPGKAPKVLIY | GFTVSSNYMTWVRQAPGKGLEWV |
| | KASSLESGVPSRFSGSGSGTEFTL | SVIYSGGSTYYADSVKGRFTISRDN |
| | TINSLQPDDFATYYCQQYKSFSLTF | SKNTLYLQMNSLRAEDTAVYYCARN |
| | GQGTKLEIK | WGDYWGQGTLVTVSS |
| | (SEQ ID NO: 290) | (SEQ ID NO: 291) |
| 11A1 | DIQMTQSPSTLSASVGDRVTITCRA | QVQLQESGPGLVKPSGTLSLTCTVS |
| | SQSISSWLAWYQQKPGKAPKVLIY | GGSIDYYFWNWFRQSPVKGLEWIG |
| | KASTLESGVPSRFSGSGSGTEFTL | HVYDIGNTKYNPSLKSRVTISIDTSE |
| | TISSLQPDDFATYYCQQYNSYSYTF | NQFSLKLNSVTAADTAVYYCARGEG |
| | GHGTKLEIK | AIDAFDIWGQGTMVTVSS |
| | (SEQ ID NO: 292) | (SEQ ID NO: 293) |
| 11C1 | DIQMTQSPSILSASVGDRVTITCRA | QVQLQESGPGLVKPSETLSLNCTVS |
| | SQSVSSWLAWYQQKPGKAPKVLIY | GGSISYYYWTWIRQPPGKGLEWIG |
| | KASSLESGVPSRFSGTGSGTEFTL | HVIYSGTTNYNPSLKSRVTISVDTSK |
| | TISSLQSDDFATYYCQQYNTYSHTF | NQFSLKLNSVTAADTAVYYCVRAEG |
| | GQGTKLEIK | SIDAFDLWGQGTMVTVSS |
| | (SEQ ID NO: 294) | (SEQ ID NO: 295) |
| 11D1 | AIQMTQSPSSLSASVGDRVTITCRA | QVQLVESGGGVVQPGRSLRLSCVA |
| | SQGIRNDLGWYQQKPGKAPKLLIY | SGFTFSDYGIHWVRQAPGMGQEW |
| | AASSLQSGVPSRFSGSGSGTDFTL | VAVIWYDGSiKKYSDSVKGRFIISRD |
| | TISSLQPEDFATYYCLQDYNYPFTF | NSENTVYLQMNSLRGEDTAIYYCAR |
| | GPGTKVDIK | DEVGtfGAFDFWGQGTKVTVSS |
| | (SEQ ID NO: 296) | (SEQ ID NO: 297) |
| 11E1 | DIQMTQSPSSLSASVGDSITITCRA | QVQLQESGPGLVKPLQTLSLTCTVS |
| | SQDIDNYLAWYQQKTGKVPKVLIY | GGSISSdgYYWSWIRQNPGKGLEWI |
| | AASALQSGVPSRFSGSGSGTDFTL | GYMYYSGSTYYNPSLKSRVTISVDT |
| | TISSLQPEDVATYYCQNYNSGPRTF | SKNQFSLKLRSVTAADTAVYYCTRD |
| | GQGTKVEIK | FGWYFDLWGRGTLVTVSS |
| | (SEQ ID NO: 298) | (SEQ ID NO: 299) |
| 12A2 | DIQMTQSPSSLSASVGDRVTITCRA | QVQLQESGPGLVKPSQSLSLTCSVS |
| | SQDISNYLTWYQQKPGRVPEVLIY | GGSVSSdgYYWSWIRQHPGKGLEW |
| | AASALQSGVPSRFSGSGSGTDFTL | IGYIYYRRITDYNPSLKSRVNISLDTS |
| | TISSLQPEDVATYYCQNYNSAPRTF | KNQFSLKLSSVTAADTAVYYCARDF |
| | GQGTKVEIK | GWYFDLWGRGTLVAVSS |
| | (SEQ ID NO: 300) | (SEQ ID NO: 301) |
| 12C4 | DIVMTQSPLSLPVTPGEPASISCRS | QVQLVQSGAEVKKPGASVKVSCKA |
| | SQSLLHSNGYNYLDWYLQKPGQS | SGYTFTGYYLHWVRQAPGQGLEW |
| | PQVLILLGSNRASGVPDRVSASGS | MGWINpNSGGTNYAQKFQGRVTMT |
| | GTDFTLKISRMQAEDVGIYYCMQTL | RDTSITTAYMELSRLRIDDTAVYYCA |
| | QTPFTFGQGTKLEIK | RDRGVtmivDGMDDWGQGTTVTVS |
| | (SEQ ID NO: 302) | S |
| | | (SEQ ID NO: 303) |
| 12C5 | DIQLTQSPSFLSASVGDRVIITCRAS | EVELVESGGGMVQPGRSLRLSCAA |
| | QGINSHLAWYQQKPGKAPKLLIYY | SGFTFSDYGMHWVRQAPGMGLEW |
| | ASTLPSGVPSRFSGSGSGTEFTLT | VTVIWYDGSnKYYADSVKGRFTISR |
| | VTSLQPEDFATYYCQQLNHYPITFG | DNSKNTVFLQMNSLRAEDTAVYYC |
| | QGTRLDIN | ARDEVGfvGAFDIWGQGTMVTVSS |
| | (SEQ ID NO: 304) | (SEQ ID NO: 305) |
| 12D3 | DIQMTQSPSSLSASVGDRVTITCRA | QVQLQESGPGLVKPSQTLSLTCTVS |
| | SQGISNYLAWYQQKPGKVPKLLIY | GGSISSdgYYWSWIRQHPGKGLEWI |
| | AASTLHSGVPSRFSGSGSGTDFTL | GYMYYSGITYHNPSLKSRVTISVDTS |
| | TISSLQPEDVATYYCQKYNSAPRTF | KNQFSLRLSSVTAADTAVYYCARDF |
| | GQGTKVEIK | GWYFDLWGRGTLVTVSS |
| | (SEQ ID NO: 306) | (SEQ ID NO: 307) |
| 12D6 | DIQMTQSPSSLSASVGDRVTITCRA | QVQLQESGPGLVKPSQTLSLTCTVS |
| | SQDISNYLAWYQQKPGKVPKLLIYA | GGSISSdaYYWSWIRQHPGKGLEWI |
| | ASTLHSGVPSRFSGSGSGTDFTLTI | GYMYYSGITYYNPSLKSRVTISVDTS |
| | SSLQPDDFAAYYCQKYNSAPRTFG | KNQFSLKLSSVTAADTAVYYCARDF |
| | QGTKVEIK | GWYFDLWGRGTLVTVSS |
| | (SEQ ID NO: 308) | (SEQ ID NO: 309) |
| 12D7 | DIQLTQSPSFLSASVGDRVSITCRA | QVQLVESGGGVVQPGRSLRLSCVA |
| | SQDISSFLAWYQQKPGKAPVLLIYV | SGFTFSDYGIHWVRQAPGMGQEW |
| | ASTLQSGVPSRFSGSGSGTEFTLT | VAVIWYDGSiKKYSDSVKGRFIISRD |
| | VSSLQPEDFATYYCQQLHVYPITFG | NSENTVYLQMNSLRGEDTAIYYCAR |
| | QGTRLEIR | DEVGtfGAFDFWGQGTKVTVSS |
| | (SEQ ID NO: 310) | (SEQ ID NO: 311) |
| 12F5 | DIVMTQTPLSLPVTPGEPASISCRS | EVQLVESGGGLVKPGGSLRLSCAA |
| | SQSLLDSDDGNtYLDWYLQKPGQS | SGFTFSNAWMSWVRQAPGKGLEW |
| | PQLLIYTLSYRASGVPDRFSGSGS | VGRIKsktGGGTTDYAAPVKGRFTIS |
| | GTDFTLKISRVEAEDVGVYYCMQRI | RDDSKNTLYLQMNSLKTEDTAVYYC |
| | EFPFTFGPGTKVDIK | TSLIVGaiSLFDYWGQGTLVTVSS |
| | (SEQ ID NO: 312) | (SEQ ID NO: 313) |
| 12H4 | DIQMTQSPSALSASVGDRVAITCRA | QVQLRESGPGLVKPSETLSLTCTIS |
| | SQTISTWLAWYQQKPGKAPKVLIY | GGSISYYFWTWIRQPPGRGLEWIG |
| | KASNLESGVPSRFSGSGSGTEFTL | QIYYSGNTNSNPSLKSRVTISIDTSK |
| | TINSLQPDDFATYYCQQYQTFSHTF | NQFSLKLTSVTVADTAVYYCVRAEG |
| | GQGTKLEIK | SIDAFDIWGQGTMVAVSS |
| | (SEQ ID NO: 314) | (SEQ ID NO: 315) |
| 8C8 | DMQMTQSPSSLSASVGDRVTLTCR | EVQLVESGGGLVKPGGSLRLSCVA |
| | ASQGISNYLAWFQLKPGKVPKLLIY | SGFTFSSYSMNWVRQFPGKGLEW |
| | AASTLQSGVPSRFSGSGSGTDFAL | VSSIStSSNYIHYADSLQGRFTISRDN |
| | TISSLQPEDVATYYCQKYNSAPLTF | AKNSLYLQMSSLRVEDTAVYYCVRD |
| | GGGTKVEIK | KGTtltnWYFDLWGRGTLVTVSS |
| | (SEQ ID NO: 316) | (SEQ ID NO: 317) |
| 8F7 | DIVMTQSPLSLPVTPGEPASISCRS | QVQLVESGGGVVQPGRSLRLSCGA |
| | SQTLVHSNGYNYLNWYLQKPGQS | SGFTFSSYGMHWVRQAPGKGLEW |
| | PQLLIYLGSNRASGVPDRFSGSGS | VAVIWYDGSnKYYADSLKGRFTISR |
| | GSDFTLKISRMEAEDVGVYYCMQA | DNSKNTLYLQMNSLRAEDTAVYYCA |
| | IQTPYTFGQGTNVEIK | RDGYSgSSDAFDIWGQGTMVTVSS |
| | (SEQ ID NO: 318) | (SEQ ID NO: 319) |
| 8F8 | DIQMTQSPSTLSASVGDRVTITCRA | QVQLQESGPGLVQPSETLSLTCTVS |
| | SQSISSWLAWYQQKPGKAPKVLIY | GGSISYYYWSWIRQPPGKGLEWIG |
| | KASNLESGVPSRFSGSGSGTEFTL | NINYMGNTIYNPSLKSRVTISVDTSK |
| | TISSLQPDDFATYYCQQYNSYSCTF | DQFSLKLTSVSAADTAVYYCVRAEG |
| | GQGTKLEIK | SIDAFDFWGQGTLVAVSL |
| | (SEQ ID NO: 320) | (SEQ ID NO: 321) |
| 9D8 | DIQMTQSPSSLSASVGDRIIFTCQA | QVQLVQSGAEVTKPGASVKVSCKA |
| | SQDINNYLHWYQQKPGKAPKLLIY | SGYIFTGYYIYWVRQAPGQGLEWM |
| | DASDWETGVPSRFSGSGSGTDFT | GWINpSSGGTNYAQKFQGRVTMAR |
| | FTISSLQPEDIATYYCQQYDHLPITF | DTSISTAYMELSSLRSDDTAVYYCA |
| | GQGTRVEIK | RDRKRevvvnFGMDVWGQGTTVTV |
| | (SEQ ID NO: 322) | ST |
| | | (SEQ ID NO: 323) |
| 9E10 | DIQMTQSPSSLSASVGDRVILTCQA | QVQLVQSGAEVTKPGASVKVSCKA |
| | SQDISNYLHWYQQKPGKAPKLLIYD | SGYTFTSHYIYWVRQAPGQGLEWM |
| | ASDLETGVPSRFSGSGSGADFTFTI | GWINpNSGGTNYAQKFQDRVTMAR |
| | SNLQPEDFATYYCQQYDHLPITFG | DTSISTAYMELSRLRSDDTAVYYCA |
| | QGTRLEIK | KDRKReyyynFGMDVWGQGTTVTV |
| | (SEQ ID NO: 324) | SA |
| | | (SEQ ID NO: 325) |
| 9E5 | DIQMTQSPSSLSASVGDRVILTCQA | QVQLVQFGVEVRKPGASVKVSCKV |
| | SQDISNYLHWYQQKPGKAPKLLIYD | SGFTFTSHYIYWVRQAPGQGLEWM |
| | ASDLETGVPSRFSGSGSGADFTFTI | GWINpNSGGTKYAQKFQDRVTMAR |
| | SNLQPEDFATYYCQQYDHLPITFG | DTSISTAYMELSRLRSDDTSVYYCV |
| | QGTRLEIK | KDRKReyyynFGMDVWGQGTTVTV |
| | (SEQ ID NO: 326) | SS |
| | | (SEQ ID NO: 327) |
| 9F4 | DIQMTQSPSSLSASVGDRVTITCQA | EVQMLESGGGLIQPGGSLRLSCKTS |
| | SQDISNYLNWYQQKPGKAPKLLIYD | GFTLSIYAIHWVRQAPGRGLEWVSS |
| | ASNLETGVPSRFSGSGSGTDFTFTI | FGqRGSSTYFADSVKGRFTISRDAS |
| | SSLQPEDIATYYCQQYDNLPYTFG | ENSLYLHMNSLRAEDTAVYYCAKEK |
| | QGTKLEIK | DWgRGFDYWGQGTLVTVSS |
| | (SEQ ID NO: 328) | (SEQ ID NO: 329) |
| 9F8 | DIVMTQSPLSLPVTPGEPASISCRS | EVQLVESGGGLVKPGGSLRLSCAA |
| | SQSLLYSNGYNYLDWYLQKPGQS | SGFTFSNYSMNWVRQAPGKGLEW |
| | PQLLIFLNSNRASGVPDRFSGSGS | VSSISsSTIYIYYADSVKGRFTISRDN |
| | GTDFTLKISRVEAEDVGVYFCMQA | AKKSLYLQMNSLRAEDTAVYYCARD |
| | LQTPLTFGGGTKVEIK | IGWevftLGFDYWGQGTQVTVSS |
| | (SEQ ID NO: 330) | (SEQ ID NO: 331) |

Also provided herein are CDR portions of antigen binding domains of antibodies to CD70 (including Chothia, Kabat CDRs, and CDR contact regions). Determination of CDR regions is well within the skill of the art. It is understood that in some embodiments, CDRs can be a combination of the Kabat and Chothia CDR (also termed “combined CRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof. Table 2 provides examples of CDR sequences provided herein.

TABLE 2
Heavy Chain

| mAb | CDRH1 | CDRH2 | CDRH3 |
|---|---|---|---|
| 31H1 | SYGFS (SEQ ID NO: 49) | GIIPIFGSANYAQK | GGSSSPFAY |
| | (Kabat); | FQG (SEQ ID NO: | (SEQ ID NO: 54) |
| | GGTFSSY (SEQ ID NO: | 52) (Kabat); | |
| | 50) (Chothia); | IPIFGS (SEQ ID | |
| | GGTFSSYGFS (SEQ ID | NO: 53) (Chothia) | |
| | NO: 51) (Extended) | | |
| 63B2 | SYGFS (SEQ ID NO: 55) | GIIPIFGTANYAQK | GGSSSPFAY |
| | (Kabat); | FQG (SEQ ID NO: | (SEQ ID NO: 60) |
| | GGTFSSY (SEQ ID NO: | 58) (Kabat); | |
| | 56) (Chothia) | IPIFGT (SEQ ID | |
| | GGTFSSYGFS | NO: 59) (Chothia) | |
| | (Extended) (SEQ ID NO: | | |
| | 57) | | |
| 40E3 | SYYWN (SEQ ID NO: 61) | YIYYSGSTNYNPS | DIRTW (SEQ ID |
| | (Kabat); | LKS (SEQ ID NO: | NO: 66) |
| | GGSISSY (SEQ ID NO: | 64) (Kabat); | |
| | 62) (Chothia); | YYSGS (SEQ ID | |
| | GGSISSYYWN (SEQ ID | NO: 65) (Chothia) | |
| | NO: 63) (Extended) | | |
| 42C3 | NSWMS (SEQ ID NO: | NIKRDGSEKYYV | DQTGSFDY (SEQ |
| | 67) (Kabat); | DSVKG (SEQ ID | ID NO: 72) |
| | GFTFRNS (SEQ ID NO: | NO: 70) (Kabat); | |
| | 68) (Chothia); | KRDGSE (SEQ ID | |
| | GFTFRNSWMS (SEQ ID | NO: 71) (Chothia) | |
| | NO: 69) (Extended) | | |
| 45F11 | VYYWS (SEQ ID NO: 73) | VYSSGNINYNPSL | GLDAFDI (SEQ ID |
| | (Kabat); | ES (SEQ ID NO: | NO: 78) |
| | DDSISVY (SEQ ID NO: | 76) (Kabat); | |
| | 74) (Chothia); | YSSGN (SEQ ID | |
| | DDSISVYYWS (SEQ ID | NO: 77) (Chothia) | |
| | NO: 75) (Extended) | | |
| 64F9 | SYAMS (SEQ ID NO: 79) | RVYSSGNINYNP | GLDAFDI (SEQ ID |
| | (Kabat); | SLES (SEQ ID | NO: 84) |
| | GFTFTSY (SEQ ID NO: | NO: 82) (Kabat); | |
| | 80) (Chothia); | YSSGN (SEQ ID | |
| | GFTFTSYAMS (SEQ ID | NO: 83) (Chothia) | |
| | NO: 81) (Extended) | | |
| 72C2 | TYAIS (SEQ ID NO: 85) | GIIPFFGTANYAQ | WELFFFDF (SEQ |
| | (Kabat); | KFQG (SEQ ID | ID NO: 90) |
| | GGTFITY (SEQ ID NO: | NO: 88) (Kabat); | |
| | 86) (Chothia); | IPFFGT (SEQ ID | |
| | GGTFITYAIS (SEQ ID | NO: 89) (Chothia) | |
| | NO: 87) (Extended) | | |
| 2F10 | YYSMN (SEQ ID NO: 91) | HISIRSSTIYFADS | GSGWYGDYFDY |
| | (Kabat); | AKG (SEQ ID NO: | (SEQ ID NO: 96) |
| | GFTFTYY (SEQ ID NO: | 94) (Kabat); | |
| | 92) (Chothia); | SIRSST (SEQ ID | |
| | GFTFTYYSMN (SEQ ID | NO: 95) (Chothia) | |
| | NO: 93) (Extended) | | |
| 4F11 | NARMGVT (SEQ ID NO: | HIFSNDEKSYSTS | IRDYYDISSYYDY |
| | 97) (Kabat); | LKS (SEQ ID NO: | (SEQ ID NO: 102) |
| | GFSLSNARM (SEQ ID | 100) (Kabat); | |
| | NO: 98) (Chothia); | FSNDE (SEQ ID | |
| | GFSLSNARMGVT (SEQ | NO: 101) (Chothia) | |
| | ID NO: 99) (Extended) | | |
| 10H10 | NHNIH (SEQ ID NO: 103) | YISRSSSTIYYAD | DHAQWYGMDV |
| | (Kabat); | SVKG (SEQ ID | (SEQ ID NO: 108) |
| | GFTFSNH (SEQ ID NO: | NO: 106) (Kabat); | |
| | 104) (Chothia); | SRSSST (SEQ ID | |
| | GFTFSNHNIH (SEQ ID | NO: 107) (Chothia) | |
| | NO: 105) (Extended) | | |
| 17G6 | SYWMS (SEQ ID NO: | SIKQDGSEKYYV | EGVNWGWRLYW |
| | 109) (Kabat); | DSVKG (SEQ ID | HFDL (SEQ ID |
| | GFTFSSY (SEQ ID NO: | NO: 112) (Kabat); | NO: 114) |
| | 110) (Chothia); | KQDGSE (SEQ ID | |
| | GFTFSSYWMS (SEQ ID | NO: 113) (Chothia) | |
| | NO: 111) (Extended) | | |
| 65E11 | SYSMN (SEQ ID NO: | HSSISRGNIYFAD | GSGWYGDYFDY |
| | 115) (Kabat); | SVKG (SEQ ID | (SEQ ID NO: 120) |
| | GFTFSSY (SEQ ID NO: | NO: 118) (Kabat); | |
| | 116) (Chothia); | SISRGN (SEQ ID | |
| | GFTFSSYSMN (SEQ ID | NO: 119) (Chothia) | |
| | NO: 117) (Extended) | | |
| P02B10 | NYAMS (SEQ ID NO: | AIRGGGGSTYYA | DFISGTWYPDY |
| | 121) (Kabat); | DSVKG (SEQ ID | (SEQ ID NO: 126) |
| | GFAFSNY (SEQ ID NO: | NO: 124) (Kabat); | |
| | 122) (Chothia); | RGGGGS (SEQ ID | |
| | GFAFSNYAMS (SEQ ID | NO: 125) (Chothia) | |
| | NO: 123) (Extended) | | |
| P07D03 | SYWIG (SEQ ID NO: | SIYPDDSDTRYSP | STVDYPGYSYFD |
| | 127) (Kabat); | SFQG (SEQ ID | Y (SEQ ID NO: |
| | GYRFTSY (SEQ ID NO: | NO: 130) (Kabat); | 132) |
| | 128) (Chothia); | YPDDSD (SEQ ID | |
| | GYRFTSYWIG (SEQ ID | NO: 131) (Chothia) | |
| | NO: 129) (Extended) | | |
| P08A02 | NYWIA (SEQ ID NO: | IIYPDGSDTRYSP | DITSWYYGEPAF |
| | 133) (Kabat); | SFQG (SEQ ID | DI |
| | GYTFTNY (SEQ ID NO: | NO: 136) (Kabat); | (SEQ ID NO: 138) |
| | 134) (Chothia); | YPDGSD (SEQ ID | |
| | GYTFTNYWIA (SEQ ID | NO: 137) (Chothia) | |
| | NO: 135) (Extended) | | |
| P08E02 | SSWIG (SEQ ID NO: | IIYPGDSDTRYSP | GLSQAMTGFGFD |
| | 139) (Kabat); | SFQG (SEQ ID | Y (SEQ ID NO: |
| | GYSFTSS (SEQ ID NO: | NO: 142) (Kabat); | 144) |
| | 140) (Chothia); | YPGDSD (SEQ ID | |
| | GYSFTSSWIG (SEQ ID | NO: 143) (Chothia) | |
| | NO: 141) (Extended) | | |
| P08F08 | SYWIG (SEQ ID NO: | IIHPDDSDTKYSP | SYLRGLWGGYF |
| | 145) (Kabat); | SFQG (SEQ ID | DY (SEQ ID NO: |
| | GYGFTSY (SEQ ID NO: | NO: 148) (Kabat); | 150) |
| | 146) (Chothia); | HPDDSD (SEQ ID | |
| | GYGFTSYWIG (SEQ ID | NO: 149) (Chothia) | |
| | NO: 147) (Extended) | | |
| P08G02 | SSWIG (SEQ ID NO: | IIYPDTSHTRYSP | ASYFDRGTGYSS |
| | 151) (Kabat); | SFQ (SEQ ID NO: | WWMDV (SEQ ID |
| | GYTFPSS (SEQ ID NO: | 154) (Kabat); | NO: 156) |
| | 152) (Chothia); | YPDTSH (SEQ ID | |
| | GYTFPSSWIG (SEQ ID | NO: 155) (Chothia) | |
| | NO: 153) (Extended) | | |
| P12B09 | QYSMS (SEQ ID NO: | AISGGGVSTYYA | DISDSGGSHWYF |
| | 157) (Kabat); | DSVKG (SEQ ID | DY (SEQ ID NO: |
| | GFTFSQY (SEQ ID NO: | NO: 160) (Kabat); | 162) |
| | 158) (Chothia); | SGGGVS (SEQ ID | |
| | GFTFSQYSMS (SEQ ID | NO: 161) (Chothia) | |
| | NO: 159) (Extended) | | |
| P12F02 | SYAMS (SEQ ID NO: | TISGTGGTTYYAD | VRAGIDPTASDV |
| | 163) (Kabat); | SVKG (SEQ ID | (SEQ ID NO: 168) |
| | GFTFSSY (SEQ ID NO: | NO: 166) (Kabat); | |
| | 164) (Chothia); | SGTGGT (SEQ ID | |
| | GFTFSSYAMS (SEQ ID | NO: 167) (Chothia) | |
| | NO: 165) (Extended) | | |
| P12G07 | NFAMS (SEQ ID NO: | GISGSGDNTYYA | DRDIGLGWYSYY |
| | 169) (Kabat); | DSVKG (SEQ ID | LDV (SEQ ID NO: |
| | GFTFNNF (SEQ ID NO: | NO: 172) (Kabat); | 174) |
| | 170) (Chothia); | SGSGDN (SEQ ID | |
| | GFTFNNFAMS (SEQ ID | NO: 173) (Chothia) | |
| | NO: 171) (Extended) | | |
| P13F04 | SYAIS (SEQ ID NO: 175) | EIIPIFGTASYAQK | AGWDDSWFDY |
| | (Kabat); | FQG (SEQ ID NO: | (SEQ ID NO: 180) |
| | GGTFSSY (SEQ ID NO: | 178) (Kabat); | |
| | 176) (Chothia); | IPIFGT (SEQ ID | |
| | GGTFSSYAIS (SEQ ID | NO: 179) (Chothia) | |
| | NO: 177) (Extended) | | |
| P15D02 | SYWIG (SEQ ID NO: | VIYPGTSETRYSP | GLSASASGYSFQ |
| | 181) (Kabat); | SFQG (SEQ ID | Y (SEQ ID NO: |
| | GYSFASY (SEQ ID NO: | NO: 184) (Kabat); | 186) |
| | 182) (Chothia); | YPGTSE (SEQ ID | |
| | GYSFASYWIG (SEQ ID | NO: 185) (Chothia) | |
| | NO: 183) (Extended) | | |
| P16C05 | DYWIG (SEQ ID NO: | MISPGGSTTIYRP | MYTGGYGGSWY |
| | 187) (Kabat); | SFQG (SEQ ID | FDY (SEQ ID NO: |
| | GYSFTDY (SEQ ID NO: | NO: 190) (Kabat); | 192) |
| | 188) (Chothia); | SPGGST (SEQ ID | |
| | GYSFTDYWIG (SEQ ID | NO: 191) (Chothia) | |
| | NO: 189) (Extended) | | |
| 10A1 | YYYWT (SEQ ID NO: | HIYYSGSTNYNPS | AEGSIDAFDF |
| | 332) (Kabat); | LKS (SEQ ID NO: | (SEQ ID NO: 337) |
| | GGSISYY (SEQ ID NO: | 335) (Kabat); | |
| | 333) (Chothia); | YYSGS (SEQ ID | |
| | GGSISYYYWT (SEQ ID | NO: 336) (Chothia) | |
| | NO: 334) (Extended) | | |
| 10E2 | SNYMT (SEQ ID NO: | VIYSGGSTYYADS | NWGDYW (SEQ |
| | 338) (Kabat); | VKG (SEQ ID NO: | ID NO: 343) |
| | GFTVSSN (SEQ ID NO: | 341) (Kabat); | |
| | 339) (Chothia); | YSGGS (SEQ ID | |
| | GFTVSSNYMT (SEQ ID | NO: 342) (Chothia) | |
| | NO: 340) (Extended) | | |
| 11A1 | YYFWN (SEQ ID NO: | HVYDIGNTKYNP | GEGAIDAFDI |
| | 344) (Kabat); | SLKS (SEQ ID | (SEQ ID NO: 349) |
| | GGSIDYY (SEQ ID NO: | NO: 347) (Kabat); | |
| | 345) (Chothia); | YDIGN (SEQ ID | |
| | GGSIDYYFWN (SEQ ID | NO: 348) (Chothia) | |
| | NO: 346) (Extended) | | |
| 11C1 | YYYWT (SEQ ID NO: | HVIYSGTTNYNPS | AEGSIDAFDL |
| | 350) (Kabat); | LKS (SEQ ID NO: | (SEQ ID NO: 355) |
| | GGSISYY (SEQ ID NO: | 353) (Kabat); | |
| | 351) (Chothia); | IYSGT (SEQ ID | |
| | GGSISYYYWT (SEQ ID | NO: 354) (Chothia) | |
| | NO: 352) (Extended) | | |
| 11D1 | DYGIH (SEQ ID NO: 356) | VIWYDGSiKKYSD | DEVGtfGAFDF |
| | (Kabat); | SVKG (SEQ ID | (SEQ ID NO: 361) |
| | GFTFSDY (SEQ ID NO: | NO: 359) (Kabat); | |
| | 357) (Chothia); | WYDGSi (SEQ ID | |
| | GFTFSDYGIH (SEQ ID | NO: 360) (Chothia) | |
| | NO: 358) (Extended) | | |
| 11E1 | SdgYYWS (SEQ ID NO: | YMYYSGSTYYNP | DFGWYFDL (SEQ |
| | 362) (Kabat); | SLKS (SEQ ID | ID NO: 367) |
| | GGSISSdgY (SEQ ID | NO: 365) (Kabat); | |
| | NO: 363) (Chothia); | YYSGS (SEQ ID | |
| | GGSISSdgYYWS (SEQ | NO: 366) (Chothia) | |
| | ID NO: 364) (Extended) | | |
| 12A2 | SdgYYWS (SEQ ID NO: | YIYYRRITDYNPS | DFGWYFDL (SEQ |
| | 368) (Kabat); | LKS (SEQ ID NO: | ID NO: 373) |
| | GGSVSSdgY (SEQ ID | 371) (Kabat); | |
| | NO: 369) (Chothia); | YYRRI (SEQ ID | |
| | GGSVSSdgYYWS (SEQ | NO: 372) (Chothia) | |
| | ID NO: 370) (Extended) | | |
| 12C4 | GYYLH (SEQ ID NO: | WINpNSGGTNYA | DRGVtmivDGMD |
| | 374) (Kabat); | QKFQG (SEQ ID | D (SEQ ID NO: |
| | GYTFTGY (SEQ ID NO: | NO: 377) (Kabat); | 379) |
| | 375) (Chothia); | NpNSGG (SEQ ID | |
| | GYTFTGYYLH (SEQ ID | NO: 378) (Chothia) | |
| | NO: 376) (Extended) | | |
| 12C5 | DYGMH (SEQ ID NO: | VIWYDGSnKYYA | DEVGfvGAFDI |
| | 380) (Kabat); | DSVKG (SEQ ID | (SEQ ID NO: 385) |
| | GFTFSDY (SEQ ID NO: | NO: 383) (Kabat); | |
| | 381) (Chothia); | WYDGSn (SEQ ID | |
| | GFTFSDYGMH (SEQ ID | NO: 384) (Chothia) | |
| | NO: 382) (Extended) | | |
| 12D3 | SdgYYWS (SEQ ID NO: | YMYYSGITYHNP | DFGWYFDL |
| | 386) (Kabat); | SLKS (SEQ ID | (SEQ ID NO: 391) |
| | GGSISSdgY (SEQ ID | NO: 389) (Kabat); | |
| | NO: 387) (Chothia); | YYSGI (SEQ ID | |
| | GGSISSdgYYWS (SEQ | NO: 390) (Chothia) | |
| | ID NO: 388) (Extended) | | |
| 12D6 | SdaYYWS (SEQ ID NO: | YMYYSGITYYNP | DFGWYFDL (SEQ |
| | 392) (Kabat); | SLKS (SEQ ID | ID NO: 397) |
| | GGSISSdaY (SEQ ID | NO: 395) (Kabat); | |
| | NO: 393) (Chothia); | YYSGI (SEQ ID | |
| | GGSISSdaYYWS (SEQ | NO: 396) (Chothia) | |
| | ID NO: 394) (Extended) | | |
| 12D7 | DYGIH (SEQ ID NO: 398) | VIWYDGSiKKYSD | DEVGtfGAFDF |
| | (Kabat); | SVKG (SEQ ID | (SEQ ID NO: 403) |
| | GFTFSDY (SEQ ID NO: | NO: 401) (Kabat); | |
| | 399) (Chothia); | WYDGSi (SEQ ID | |
| | GFTFSDYGIH (SEQ ID | NO: 402) (Chothia) | |
| | NO: 400) (Extended) | | |
| 12F5 | NAWMS (SEQ ID NO: | RIKsktGGGTTDY | LIVGaiSLFDY |
| | 404) (Kabat); | AAPVKG (SEQ ID | (SEQ ID NO: 409) |
| | GFTFSNA (SEQ ID NO: | NO: 407) (Kabat); | |
| | 405) (Chothia); | KsktGGGT (SEQ | |
| | GFTFSNAWMS (SEQ ID | ID NO: 408) | |
| | NO: 406) (Extended) | (Chothia) | |
| 12H4 | YYFWT (SEQ ID NO: | QIYYSGNTNSNP | AEGSIDAFDI |
| | 410) (Kabat); | SLKS (SEQ ID | (SEQ ID NO: 415) |
| | GGSISYY (SEQ ID NO: | NO: 413) (Kabat); | |
| | 411) (Chothia); | YYSGN (SEQ ID | |
| | GGSISYYFWT (SEQ ID | NO: 414) (Chothia) | |
| | NO: 412) (Extended) | | |
| 8C8 | SYSMN (SEQ ID NO: | SIStSSNYIHYADS | DKGTtltnWYFDL |
| | 416) (Kabat); | LQG (SEQ ID NO: | (SEQ ID NO: 421) |
| | GFTFSSY (SEQ ID NO: | 419) (Kabat); | |
| | 417) (Chothia); | StSSNY (SEQ ID | |
| | GFTFSSYSMN (SEQ ID | NO: 420) (Chothia) | |
| | NO: 418) (Extended) | | |
| 8F7 | SYGMH (SEQ ID NO: | VIWYDGSnKYYA | DGYSgssDAFDI |
| | 422) (Kabat); | DSLKG (SEQ ID | (SEQ ID NO: 427) |
| | GFTFSSY (SEQ ID NO: | NO: 425) (Kabat); | |
| | 423) (Chothia); | WYDGSn (SEQ ID | |
| | GFTFSSYGMH (SEQ ID | NO: 426) (Chothia) | |
| | NO: 424) (Extended) | | |
| 8F8 | YYYWS (SEQ ID NO: | NINYMGNTIYNPS | AEGSIDAFDF |
| | 428) (Kabat); | LKS (SEQ ID NO: | (SEQ ID NO: 433) |
| | GGSISYY (SEQ ID NO: | 431) (Kabat); | |
| | 429) (Chothia); | NYMGN (SEQ ID | |
| | GGSISYYYWS (SEQ ID | NO: 432) (Chothia) | |
| | NO: 430) (Extended) | | |
| 9D8 | GYYIY (SEQ ID NO: 434) | WINpSSGGTNYA | DRKReyyynFGMD |
| | (Kabat); | QKFQG (SEQ ID | V (SEQ ID NO: |
| | GYIFTGY (SEQ ID NO: | NO: 437) (Kabat); | 439) |
| | 435) (Chothia); | NpSSGG (SEQ ID | |
| | GYIFTGYYIY (SEQ ID | NO: 438) (Chothia) | |
| | NO: 436) (Extended) | | |
| 9E10 | SHYIY (SEQ ID NO: 440) | WINpNSGGTNYA | DRKReyyynFGMD |
| | (Kabat); | QKFQD (SEQ ID | V (SEQ ID NO: |
| | GYTFTSH (SEQ ID NO: | NO: 443) (Kabat); | 4454) |
| | 441) (Chothia); | NpNSGG (SEQ ID | |
| | GYTFTSHYIY (SEQ ID | NO: 444) (Chothia) | |
| | NO: 442) (Extended) | | |
| 9E5 | SHYIY (SEQ ID NO: 446) | WINpNSGGTKYA | DRKReyyynFGMD |
| | (Kabat); | QKFQD (SEQ ID | V (SEQ ID NO: |
| | GFTFTSH (SEQ ID NO: | NO: 449) (Kabat); | 451) |
| | 447) (Chothia); | NpNSGG (SEQ ID | |
| | GFTFTSHYIY (SEQ ID | NO: 450) (Chothia) | |
| | NO: 448) (Extended) | | |
| 9F4 | IYAIH (SEQ ID NO: 452) | SFGgRGSSTYFA | EKDWgRGFDY |
| | (Kabat); | DSVKG (SEQ ID | (SEQ ID NO: 457) |
| | GFTLSIY (SEQ ID NO: | NO: 455) (Kabat); | |
| | 453) (Chothia); | GgRGSS (SEQ ID | |
| | GFTLSIYAIH (SEQ ID | NO: 456) (Chothia) | |
| | NO: 454) (Extended) | | |
| 9F8 | NYSMN (SEQ ID NO: | SISsSTIYIYYADS | DIGWevftLGFDY |
| | 458) (Kabat); | VKG (SEQ ID NO: | (SEQ ID NO: 463) |
| | GFTFSNY (SEQ ID NO: | 461) (Kabat); | |
| | 459) (Chothia); | SsSTIY (SEQ ID | |
| | GFTFSNYSMN (SEQ ID | NO: 462) (Chothia) | |
| | NO: 460) (Extended) | | |

Light Chain

| mAb | CDRL1 | CDRL2 | CDRL3 |
|---|---|---|---|
| 31H1 | RSSQSLVHSDGNTYLS | KISNRFS (SEQ | MQATQFPLT |
| | (SEQ ID NO: 193); | ID NO: 194) | (SEQ ID NO: 195) |
| 63B2 | RSSQSLVHSDGNTYLS | KISNRFS (SEQ | MQATQFPLT |
| | (SEQ ID NO: 196); | ID NO: 197) | (SEQ ID NO: 198) |
| 40E3 | RASQGISNYLA (SEQ ID | AASSLQS (SEQ | QQYNSYPLT |
| | NO: 199); | ID NO: 200) | (SEQ ID NO: 201) |
| 42C3 | RSSQSLVYSDENTYLN | QVSNRDS (SEQ | MQGTYWPPT |
| | (SEQ ID NO: 202); | ID NO: 203) | (SEQ ID NO: 204) |
| 45F11 | RASQSVSSSLA (SEQ | GASTRAT (SEQ | QQYINWPH |
| | ID NO: 205); | ID NO: 206) | (SEQ ID NO: 207) |
| 64F9 | QASQDISNYLN (SEQ ID | GASNLET (SEQ | QQYDNFPIT |
| | NO: 208); | ID NO: 209) | (SEQ ID NO: 210) |
| 72C2 | RASQSVSSNLA (SEQ | SASTRAS (SEQ | QQYDNWPPLT |
| | ID NO: 211); | ID NO: 212) | (SEQ ID NO: 213) |
| 2F10 | RASQSVSSSYLA (SEQ | GASSRAT (SEQ | QQYGSSPLT |
| | ID NO: 214); | ID NO: 215) | (SEQ ID NO: 216) |
| 4F11 | RASQDISNYLA (SEQ ID | AASSLQS (SEQ | LQLNSFPFT |
| | NO: 217); | ID NO: 218) | (SEQ ID NO: 219) |
| 10H10 | RASQGISSWLA (SEQ ID | AASSLQS (SEQ | QQAFSFPFT |
| | NO: 220); | ID NO: 221) | (SEQ ID NO: 222) |
| 17G6 | KSSQSVLYSYNNKNYV | WASTRES (SEQ | QQYYSTLT (SEQ |
| | A (SEQ ID NO: 223); | ID NO: 224) | ID NO: 225) |
| 65E11 | RASQSVSSSYLA (SEQ | DASSRAT (SEQ | QQYGSSPLT |
| | ID NO: 226); | ID NO: 227) | (SEQ ID NO: 228) |
| P02B10 | SGSSSNIGSNYVY (SEQ | RNNQRPS (SEQ | AAWDDSLSGVV |
| | ID NO: 229); | ID NO: 230) | (SEQ ID NO: 231) |
| P07D03 | SGSRSNIGSNYVY (SEQ | RNNQRPS (SEQ | ASWDGSLSAVV |
| | ID NO: 232); | ID NO: 233) | (SEQ ID NO: 234) |
| P08A02 | SGSSSNIGSNYVY (SEQ | RNNQRPS (SEQ | ATWDDSLGSPV |
| | ID NO: 235); | ID NO: 236) | (SEQ ID NO: 237) |
| P08E02 | RASQSISRYLN (SEQ ID | AASILQT (SEQ ID | QQSYSTTMWT |
| | NO: 238); | NO: 239) | (SEQ ID NO: 240) |
| P08F08 | SGSSSNIGSNYVN (SEQ | GDYQRPS (SEQ | ATRDDSLSGSVV |
| | ID NO: 241); | ID NO: 242) | (SEQ ID NO: 243) |
| P08G02 | RASQSIYDYLH (SEQ ID | DASNLQS (SEQ | QQSYTTPLFT |
| | NO: 244); | ID NO: 245) | (SEQ ID NO: 246) |
| P12B09 | RASQYIGRYLN (SEQ ID | GATSLAS (SEQ | QQSYSTTSPT |
| | NO: 247); | ID NO: 248) | (SEQ ID NO: 249) |
| P12F02 | SGSTSNIGRNYVY (SEQ | RTNQRPS (SEQ | AAWDDSLSGRV |
| | ID NO: 250); | ID NO: 251) | (SEQ ID NO: 252) |
| P12G07 | SGSSSNIGSNYVY (SEQ | MNNQRPS (SEQ | AAWDDSLSAVV |
| | ID NO: 253); | ID NO: 254) | (SEQ ID NO: 255) |
| P13F04 | SGSNSNIGTNYVS (SEQ | RSSRRPS (SEQ | AAWDGSLSGHWV |
| | ID NO: 256); | ID NO: 257) | (SEQ ID NO: 258) |
| P15D02 | RASQSIDTYLN (SEQ ID | SASSLHS (SEQ | QQSYSTTAWT |
| | NO: 259); | ID NO: 260) | (SEQ ID NO: 261) |
| P16C05 | RASQSIGQSLN (SEQ ID | GASSLQS (SEQ | QQSYSTPIT |
| | NO: 262); | ID NO: 263) | (SEQ ID NO: 264) |
| 10A1 | RASQSISTWLA (SEQ ID | KASSLES (SEQ | QQYKSYSHT |
| | NO: 464); | ID NO: 465) | (SEQ ID NO: 466) |
| 10E2 | RASQSISSWLA (SEQ ID | KASSLES (SEQ | QQYKSFSLT |
| | NO: 467); | ID NO: 468) | (SEQ ID NO: 469) |
| 11A1 | RASQSISSWLA (SEQ ID | KASTLES(SEQ ID | QQYNSYSYT |
| | NO: 470); | NO: 471) | (SEQ ID NO: 472) |
| 11C1 | RASQSVSSWLA (SEQ | KASSLES (SEQ | QQYNTYSHT |
| | ID NO: 473); | ID NO: 474) | (SEQ ID NO: 475) |
| 11D1 | RASQGIRNDLG (SEQ ID | AASSLQS (SEQ | LQDYNYPFT |
| | NO: 476); | ID NO: 477) | (SEQ ID NO: 478) |
| 11E1 | RASQDIDNYLA (SEQ ID | AASALQS (SEQ | QNYNSGPRT |
| | NO: 479); | ID NO: 480) | (SEQ ID NO: 481) |
| 12A2 | RASQDISNYLT (SEQ ID | AASALQS (SEQ | QNYNSAPRT |
| | NO: 482); | ID NO: 483) | (SEQ ID NO: 484) |
| 12C4 | RSSQSLLHSNGYNYLD | LGSNRAS (SEQ | MQTLQTPFT |
| | (SEQ ID NO: 485); | ID NO: 486) | (SEQ ID NO: 487) |
| 12C5 | RASQGINSHLA (SEQ ID | YASTLPS (SEQ ID | QQLNHYPIT |
| | NO: 488); | NO: 489) | (SEQ ID NO: 490) |
| 12D3 | RASQGISNYLA (SEQ ID | AASTLHS (SEQ | QKYNSAPRT |
| | NO: 491); | ID NO: 492) | (SEQ ID NO: 493) |
| 12D6 | RASQDISNYLA (SEQ ID | AASTLHS (SEQ | QKYNSAPRT |
| | NO: 494); | ID NO: 495) | (SEQ ID NO: 496) |
| 12D7 | RASQDISSFLA (SEQ ID | VASTLQS (SEQ | QQLHVYPIT |
| | NO: 497); | ID NO: 498) | (SEQ ID NO: 499) |
| 12F5 | RSSQSLLDSDDGNtYLD | TLSYRAS (SEQ | MQRIEFPFT |
| | (SEQ ID NO: 500); | ID NO: 501) | (SEQ ID NO: 502) |
| 12H4 | RASQTISTWLA (SEQ ID | KASNLES (SEQ | QQYQTFSHT |
| | NO: 503); | ID NO: 504) | (SEQ ID NO: 505) |
| 8C8 | RASQGISNYLA (SEQ ID | AASTLQS (SEQ | QKYNSAPLT |
| | NO: 506); | ID NO: 507) | (SEQ ID NO: 508) |
| 8F7 | RSSQTLVHSNGYNYLN | LGSNRAS (SEQ | MQAIQTPYT |
| | (SEQ ID NO: 509); | ID NO: 510) | (SEQ ID NO: 511) |
| 8F8 | RASQSISSWLA (SEQ ID | KASNLES (SEQ | QQYNSYSCT |
| | NO: 512); | ID NO: 513) | (SEQ ID NO: 514) |
| 9D8 | QASQDINNYLH (SEQ ID | DASDWET (SEQ | QQYDHLPIT |
| | NO: 515); | ID NO: 516) | (SEQ ID NO: 517) |
| 9E10 | QASQDISNYLH (SEQ ID | DASDLET (SEQ | QQYDHLPIT |
| | NO: 518); | ID NO: 519) | (SEQ ID NO: 520) |
| 9E5 | QASQDISNYLH (SEQ ID | DASDLET (SEQ | QQYDHLPIT |
| | NO: 521); | ID NO: 522) | (SEQ ID NO: 523) |
| 9F4 | QASQDISNYLN (SEQ ID | DASNLET (SEQ | QQYDNLPYT |
| | NO: 524); | ID NO: 525) | (SEQ ID NO: 526) |
| 9F8 | RSSQSLLYSNGYNYLD | LNSNRAS (SEQ | MQALQTPLT |
| | (SEQ ID NO: 527); | ID NO: 528) | (SEQ ID NO: 529) |

In some embodiments, the present invention provides an antibody that binds to CD70 and competes with the antibody as described herein, including 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02, P16C05, 10A1, 10E2, 11A1, 11C1, 11D1, 11E1, 12A2, 12C4, 12C5, 12D3, 12D6, 12D7, 12F5, 12H4, 8C8, 8F7, 8F8, 9D8, 9E10, 9E5, 9F4 or 9F8.

In some embodiments, the invention also provides CDR portions of antibodies to CD70 antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (K_{D}) of the CD70 antibody as described herein to CD70 (such as human CD70 (e.g., (SEQ ID NO: 278)) can be about 0.001 to about 5000 nM. In some embodiments, the binding affinity is about any of 5000 nM, 4500 nM, 4000 nM, 3500 nM, 3000 nM, 2500 nM, 2000 nM, 1789 nM, 1583 nM, 1540 nM, 1500 nM, 1490 nM, 1064 nM, 1000 nM, 933 nM, 894 nM, 750 nM, 705 nM, 678 nM, 532 nM, 500 nM, 494 nM, 400 nM, 349 nM, 340 nM, 353 nM, 300 nM, 250 nM, 244 nM, 231 nM, 225 nM, 207 nM, 200 nM, 186 nM, 172 nM, 136 nM, 113 nM, 104 nM, 101 nM, 100 nM, 90 nM, 83 nM, 79 nM, 74 nM, 54 nM, 50 nM, 45 nM, 42 nM, 40 nM, 35 nM, 32 nM, 30 nM, 25 nM, 24 nM, 22 nM, 20 nM, 19 nM, 18 nM, 17 nM, 16 nM, 15 nM, 12 nM, 10 nM, 9 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.001 nM. In some embodiments, the binding affinity is less than about any of 5000 nM, 4000 nM, 3000 nM, 2000 nM, 1000 nM, 900 nM, 800 nM, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

Bispecific antibodies, monoclonal antibodies that have binding specificities for at least two different antigens, can be prepared using the antibodies disclosed herein. Methods for making bispecific antibodies are known in the art (see, e.g., Suresh et al., Methods in Enzymology 121:210, 1986). Traditionally, the recombinant production of bispecific antibodies was based on the coexpression of two immunoglobulin heavy chain-light chain pairs, with the two heavy chains having different specificities (Millstein and Cuello, Nature 305, 537-539, 1983). Accordingly, in one aspect, provided is a bispecific antibody wherein the bispecific antibody is a full-length human antibody, comprising a first antibody variable domain of the bispecific antibody specifically binding to a target antigen (e.g., CD70), and comprising a second antibody variable domain of the bispecific antibody capable of recruiting the activity of a human immune effector cell by specifically binding to an effector antigen located on the human immune effector cell.

The human immune effector cell can be any of a variety of immune effector cells known in the art. For example, the immune effector cell can be a member of the human lymphoid cell lineage, including, but not limited to, a T cell (e.g., a cytotoxic T cell), a B cell, and a natural killer (NK) cell. The immune effector cell can also be, for example without limitation, a member of the human myeloid lineage, including, but not limited to, a monocyte, a neutrophilic granulocyte, and a dendritic cell. Such immune effector cells may have either a cytotoxic or an apoptotic effect on a target cell or other desired effect upon activation by binding of an effector antigen.

The effector antigen is an antigen (e.g., a protein or a polypeptide) that is expressed on the human immune effector cell. Examples of effector antigens that can be bound by the heterodimeric protein (e.g., a heterodimeric antibody or a bispecific antibody) include, but are not limited to, human CD3 (or CD3 (Cluster of Differentiation) complex), CD16, NKG2D, NKp46, CD2, CD28, CD25, CD64, and CD89.

The target cell can be a cell that is native or foreign to humans. In a native target cell, the cell may have been transformed to be a malignant cell or pathologically modified (e.g., a native target cell infected with a virus, a plasmodium, or a bacterium). In a foreign target cell, the cell is an invading pathogen, such as a bacterium, a plasmodium, or a virus.

The target antigen is expressed on a target cell in a diseased condition (e.g., an inflammatory disease, a proliferative disease (e.g., cancer), an immunological disorder, a neurological disease, a neurodegenerative disease, an autoimmune disease, an infectious disease (e.g., a viral infection or a parasitic infection), an allergic reaction, a graft-versus-host disease or a host-versus-graft disease). A target antigen is not effector antigen. In some embodiments, the target antigen is CD70.

In some embodiments, provided is a bispecific antibody, wherein the bispecific antibody is a full-length antibody, comprising a first antibody variable domain of the bispecific antibody specifically binding to a target antigen, and comprising a second antibody variable domain of the bispecific antibody capable of recruiting the activity of a human immune effector cell by specifically binding to an effector antigen located on the human immune effector cell, wherein the first antibody variable domain comprises a heavy chain variable (VH) region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 289, 291, 293, 295, 297, 299, 301, 303, 305, 307, 309, 311, 313, 315, 317, 319, 321, 323, 325, 327, 329 or 331; and/or a light chain variable (VL) region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 288, 290, 292, 294, 296, 298, 300, 302, 304, 306, 308, 310, 312, 314, 316, 318, 320, 322, 324, 326, 328 or 330.

In some embodiments, provided is a bispecific antibody, wherein the bispecific antibody is a full-length antibody, comprising a first antibody variable domain of the bispecific antibody specifically binding to a target antigen, and comprising a second antibody variable domain of the bispecific antibody capable of recruiting the activity of a human immune effector cell by specifically binding to an effector antigen located on the human immune effector cell, wherein the first antibody variable domain comprises (a) a heavy chain variable (VH) region comprising (i) a VH complementarity determining region one (CDR1) comprising the sequence shown in SEQ ID NO: 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 332, 333, 334, 338, 339, 340, 344, 345, 346, 350, 351, 352, 356, 357, 358, 362, 363, 364, 368, 369, 370, 374, 375, 376, 380, 381, 382, 386, 387, 388, 392, 393, 394, 398, 399, 400, 404, 405, 406, 410, 411, 412, 416, 437, 418, 422, 423, 424, 428, 429, 430, 434, 435, 436, 440, 441, 442, 446, 447, 448, 452, 453, 454, 458, 459 or 460; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 335, 336, 341, 342, 347, 348, 353, 354, 359, 360, 365, 366, 371, 372, 377, 378, 383, 384, 389, 390, 395, 396, 401, 402, 407, 408, 413, 414, 419, 420, 425, 426, 431, 432, 437, 438, 443, 444, 449, 450, 455, 456, 461 or 462; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 337, 343, 349, 355, 361, 367, 373, 379, 385, 391, 397, 403, 409, 415, 421, 427, 433, 439, 445, 451, 457 or 463; and/or a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 464, 467, 470, 473, 476, 479, 482, 485, 488, 491, 494, 497, 500, 503, 506, 509, 512, 515, 518, 521, 524 or 527; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 465, 468, 471, 474, 477, 480, 483, 486, 489, 492, 495, 498, 501, 504, 507, 510, 513, 516, 519, 522, 525 or 528; and (iii) a VL CDR3 comprising the sequence shown in SEQ ID NO: 195, 198, 201, 204, 207, 210, 213, 216, 219, 222, 225, 228, 231, 234, 237, 240, 243, 246, 249, 252, 255, 258, 261, 264, 466, 469, 472, 475, 478, 481, 484, 487, 490, 493, 496, 499, 502, 505, 508, 511, 514, 517, 520, 523, 526 or 529.

In some embodiments, the second antibody variable domain comprises a heavy chain variable (VH) region comprising a VH CDR1, VH CDR2, and VH CDR3 of the VH sequence shown in SEQ ID NO: 266; and/or a light chain variable (VL) region comprising VL CDR1, VL CDR2, and VL CDR3 of the VL sequence shown in SEQ ID NO: 265.

In some embodiments, the second antibody variable domain comprises (a) a heavy chain variable (VH) region comprising (i) a VH complementary determining region one (CDR1) comprising the sequence shown in SEQ ID NO: 267, 268, or 269; (ii) a VH CDR2 comprising the sequence shown in SEQ ID NO: 270 or 271; and iii) a VH CDR3 comprising the sequence shown in SEQ ID NO: 272; and/or (b) a light chain variable (VL) region comprising (i) a VL CDR1 comprising the sequence shown in SEQ ID NO: 273; (ii) a VL CDR2 comprising the sequence shown in SEQ ID NO: 274; and (iii) a VL CDR3 comprising the sequence shown in SEQ ID NO: 275.

Table 3 shows the specific amino acid and nucleic acid sequences of the second antibody variable domain, which is specific to CD3. In Table 3, the underlined sequences are CDR sequences according to Kabat and in bold according to Chothia.

TABLE 3

| mAb | Light Chain | Heavy Chain |
|---|---|---|
| h2B4_ | DIVMTQSPDSLAVSLGERATIN | EVQLVESGGGLVQPGGSLRL |
| HNPS_ | CKSSQSLFNVRSRKNYLAWYQQ | SCAASGFTFSDYYMTWVRQA |
| VL_TK | KPGQPPKLLISWASTRESGVPD | PGKGLEWVAFIRNRARGYTS |
| | RFSGSGSGTDFTLTISSLQAED | DHNPSVKGRFTISRDNAKNS |
| | VAVYYCKQSYDLFTFGSGTKLE | LYLQMNSLRAEDTAVYYCAR |
| | IK (SEQ ID NO: 265) | DRPSYYVLDYWGQGTTVTVS |
| | | S (SEQ ID NO: 266) |
| h2B4_ | GACATTGTGATGACTCAATCCC | GAAGTCCAACTTGTCGAATC |
| HNPS_ | CCGACTCCCTGGCTGTGTCCCT | GGGAGGAGGCCTTGTGCAAC |
| VL_TK | CGGCGAACGCGCAACTATCAAC | CCGGTGGATCCCTGAGGCTG |
| | TGTAAAAGCAGCCAGTCCCTGT | TCATGCGCGGCCTCGGGCTT |
| | TCAACGTCCGGTCGAGGAAGAA | CACCTTTTCCGATTACTACA |
| | CTACCTGGCCTGGTATCAGCAG | TGACCTGGGTCAGACAGGCC |
| | AAACCTGGGCAGCCGCCGAAG | CCTGGAAAGGGGTTGGAATG |
| | CTTCTGATCTCATGGGCCTCAA | GGTGGCATTCATCCGGAATA |
| | CTCGGGAAAGCGGAGTGCCAG | GAGCCCGCGGATACACTTCC |
| | ATAGATTCTCCGGATCTGGCTC | GACCACAACCCCAGCGTGAA |
| | CGGAACCGACTTCACCCTGACG | GGGGCGGTTCACCATTAGCC |
| | ATTTCGAGCTTGCAAGCGGAGG | GCGACAACGCCAAGAACTCC |
| | ATGTGGCCGTGTACTACTGCAA | CTCTACCTCCAAATGAACAG |
| | GCAGTCCTACGACCTCTTCACC | CCTGCGGGCGGAGGATACCG |
| | TTTGGTTCGGGCACCAAGCTGG | CTGTGTACTACTGCGCCCGC |
| | AGATCAAA | GACCGGCCGTCCTACTATGT |
| | (SEQ ID NO: 276) | GCTGGACTACTGGGGCCAGG |
| | | GTACTACGGTCACCGTCTCC |
| | | TCA (SEQ ID NO: 277) |

Table 4 shows the examples of CDR sequences of the second antibody variable domain, which is specific to CD3.

TABLE 4

| | | | |
|---|---|---|---|
| mAb | CDRH1 | CDRH2 | CDRH3 |

Heavy Chain

| | | | |
|---|---|---|---|
| h2B4_ | SDYYMT | RNRARGYT | DRPSYYVL |
| HNPS_ | (SEQ ID NO: 267) | (SEQ ID NO: 270) | DY |
| VL_TK | (Kabat); | (Kabat) | (SEQ ID |
| | GFTFSDY | FIRNRARGYTSDHNPS | NO: 272) |
| | (SEQ ID NO: 268) | VKG | |
| | (Chothia); | (SEQ ID NO: 271) | |
| | GFTFSDYYMT | (Extended) | |
| | (SEQ ID NO: 269) | | |
| | (Extended) | | |

Light Chain

| | | | |
|---|---|---|---|
| h2B4_ | KSSQSLFNVRSRKN | WASTRES | KQSYDLFT |
| HNPS_ | YLA | (SEQ ID NO: 274) | (SEQ ID |
| VL_TK | (SEQ ID NO: 273) | | NO: 275) |

In some embodiments, a bispecific antibody provided herein which contains a CD3-specific variable domain having an anti-CD3 sequence as provided in U.S. Publication No. 20160297885, which is hereby incorporated by reference for all purposes.

According to one approach to making bispecific antibodies, antibody variable domains with the desired binding specificities (antibody-antigen combining sites) are fused to immunoglobulin constant region sequences. The fusion preferably is with an immunoglobulin heavy chain constant region, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In another approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

In another approach, the bispecific antibodies are composed of amino acid modification in the first hinge region in one arm, and the substituted/replaced amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second immunoglobulin-like Fc region (e.g., a hinge region and/or a CH3 region). In this approach, the bispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545).

In another approach, the bispecific antibodies can be generated using a glutamine-containing peptide tag engineered to the antibody directed to an epitope (e.g., CD70) in one arm and another peptide tag (e.g., a Lys-containing peptide tag or a reactive endogenous Lys) engineered to a second antibody directed to a second epitope in another arm in the presence of transglutaminase. This approach is described in International Patent Application No. PCT/IB2011/054899 (WO2012/059882).

In some embodiments, the heterodimeric protein (e.g., bispecific antibody) as described herein comprises a full-length human antibody, wherein a first antibody variable domain of the bispecific antibody specifically binding to a target antigen (e.g., CD70), and comprising a second antibody variable domain of the bispecific antibody capable of recruiting the activity of a human immune effector cell by specifically binding to an effector antigen (e.g., CD3) located on the human immune effector cell, wherein the first and second antibody variable domain of the heterodimeric protein comprise amino acid modifications at positions 223, 225, and 228 (e.g., (C223E or C223R), (E225E or E225R), and (P228E or P228R)) in the hinge region and at position 409 or 368 (e.g., K409R or L368E (EU numbering scheme)) in the CH3 region of human IgG2 (SEQ ID NO: 279).

In some embodiments, the first and second antibody variable domains of the heterodimeric protein comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1 (SEQ ID NO: 280).

In some embodiments, the first and second antibody variable domains of the heterodimeric protein comprise amino acid modifications at positions 228 (e.g., (P228E or P228R)) in the hinge region and at position 409 or 368 (e.g., R409 or L368E (EU numbering scheme)) in the CH3 region of human IgG4 (SEQ ID NO: 281).

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the CD70 monospecific antibody or the CD70 bispecific antibody (e.g., CD70-CD3) as described herein is a monoclonal antibody. For example, the CD70 monospecific antibody is a human monoclonal antibody. In another example, the CD70 arm of the CD70-CD3 bispecific antibody is a human monoclonal antibody, and the CD3 arm of the CD70-CD3 bispecific antibody is a humanized monoclonal antibody.

In some embodiments, the antibody comprises a modified constant region, such as, for example without limitation, a constant region that has increased potential for provoking an immune response. For example, the constant region may be modified to have increased affinity to an Fc gamma receptor such as, e.g., FcγRI, FcγRIIA, or FcγIII.

In some embodiments, the antibody comprises a modified constant region, such as a constant region that is immunologically inert, that is, having a reduced potential for provoking an immune response. In some embodiments, the constant region is modified as described in Eur. J. Immunol., 29:2613-2624, 1999; PCT Application No. PCT/GB99/01441; and/or UK patent application Ser. No. 98099518. The Fc can be human IgG1, human IgG2, human IgG3, or human IgG4. The Fc can be human IgG2 containing the mutation A330P331 to S330S331 (IgG2Δa), in which the amino acid residues are numbered with reference to the wild type IgG2 sequence. Eur. J. Immunol., 29:2613-2624, 1999. In some embodiments, the antibody comprises a constant region of IgG₄ comprising the following mutations (Armour et al., Molecular Immunology 40 585-593, 2003): E233F234L235 to P233V234A235 (IgG4Δc), in which the numbering is with reference to wild type IgG4. In yet another embodiment, the Fc is human IgG4 E233F234L235 to P233V234A235 with deletion G236 (IgG4Δb). In another embodiment, the Fc is any human IgG4 Fc (IgG4, IgG4Δb or IgG4Δc) containing hinge stabilizing mutation S228 to P228 (Aalberse et al., Immunology 105, 9-19, 2002). In another embodiment, the Fc can be aglycosylated Fc.

In some embodiments, the constant region is aglycosylated by mutating the oligosaccharide attachment residue (such as Asn297) and/or flanking residues that are part of the glycosylation recognition sequence in the constant region. In some embodiments, the constant region is aglycosylated for N-linked glycosylation enzymatically. The constant region may be aglycosylated for N-linked glycosylation enzymatically or by expression in a glycosylation deficient host cell.

In some embodiments, the constant region has a modified constant region that removes or reduces Fc gamma receptor binding. For example, the Fc can be human IgG2 containing the mutation D265, in which the amino acid residues are numbered with reference to the wild type IgG2 sequence (SEQ ID NO: 279). Accordingly, in some embodiments, the constant region has a modified constant region having the sequence shown in SEQ ID NO: 282: ASTKGPSVFPLAPCSRSTSESTAALGCLVKDYFPEPVTVSWNSGALTSGVHTFPAVLQ SSGLYSLSSVVTVPSSNFGTQTYTCNVDHKPSNTKVDKTVERKCRVRCPRCPAPPVA GPSVFLFPPKPKDTLMISRTPEVTCVVVAVSHEDPEVQFNWYVDGVEVHNAKTKPRE EQFNSTFRVVSVLTVVHQDWLNGKEYKCKVSNKGLPSSIEKTISKTKGQPREPQVYTL PPSREEMTKNQVSLTCLVKGFYPSDIAVEWESNGQPENNYKTTPPMLDSDGSFFLYS RLTVDKSRWQQGNVFSCSVMHEALHNHYTQKSLSLSPGK. And the nucleic acid encoding the sequence shown in SEQ ID NO: 282 is shown in SEQ ID NO: 283.

In some embodiments, the constant region has a modified constant region having the sequence shown in SEQ ID NO: 284: ASTKGPSVFPLAPCSRSTSESTAALGCLVKDYFPEPVTVSWNSGALTSGVHTFPAVLQ SSGLYSLSSVVTVPSSNFGTQTYTCNVDHKPSNTKVDKTVERKCEVECPECPAPPVA GPSVFLFPPKPKDTLMISRTPEVTCVVVAVSHEDPEVQFNWYVDGVEVHNAKTKPRE EQFNSTFRVVSVLTVVHQDWLNGKEYKCKVSNKGLPSSIEKTISKTKGQPREPQVYTL PPSREEMTKNQVSLTCEVKGFYPSDIAVEWESNGQPENNYKTTPPMLDSDGSFFLYS KLTVDKSRWQQGNVFSCSVMHEALHNHYTQKSLSLSPGK. And the nucleic acid encoding the sequence shown in SEQ ID NO: 284 is shown in SEQ ID NO: 285.

The amino acid of the human Kappa constant region is shown in SEQ ID NO: 286: GTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC. And the nucleic acid encoding the sequence of SEQ ID NO: 286 is shown in SEQ ID NO: 287.

One way of determining binding affinity of antibodies to CD70 is by measuring binding affinity of the bivalent antibody to monomeric CD70 protein. The affinity of an CD70 antibody can be determined by surface plasmon resonance (Biacore™3000™ surface plasmon resonance (SPR) system, Biacore™, INC, Piscataway NJ) equipped with pre-immobilized anti-mouse Fc or anti-human Fc using HBS-EP running buffer (0.01M HEPES, pH 7.4, 0.15 NaCl, 3 mM EDTA, 0.005% v/v Surfactant P20). Monomeric 8-histidine tagged human CD70 extracellular domain (SEQ ID NO: 530) can be diluted into HBS-EP buffer to a concentration of less than 0.5 μg/mL and injected across the individual chip channels using variable contact times, to achieve two ranges of antigen density, either 50-200 response units (RU) for detailed kinetic studies or 800-1,000 RU for screening assays. Regeneration studies have shown that 25 mM NaOH in 25% v/v ethanol effectively removes the bound CD70 protein while keeping the activity of CD70 antibodies on the chip for over 200 injections. Typically, serial dilutions (spanning concentrations of 0.1-10× estimated K_{D}) of purified 8-histidine tagged CD70 samples (SEQ ID NO: 530) are injected for 1 min at 100 μL/minute and dissociation times of up to 2 hours are allowed. The concentrations of the CD70 proteins are determined by absorbance at 280 nm based on sequence specific extinction coefficient of the 8-histidine tagged CD70 protein (SEQ ID NO: 530). Kinetic association rates (kₒₙ or kₐ) and dissociation rates (k_{off} or k_{d}) are obtained simultaneously by fitting the data globally to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values are calculated as k_{off}/kₒₙ. This protocol is suitable for use in determining binding affinity of an antibody to any monomeric CD70, including human CD70, CD70 of another mammal (such as mouse CD70, rat CD70, or primate CD70), as well as different forms of CD70 (e.g., glycosylated CD70). Binding affinity of an antibody is generally measured at 25° C., but can also be measured at 37° C.

The antibodies as described herein may be made by any method known in the art. For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for CD70, or portions thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing human CD70, a human CD70 protein, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In an alternative, the polynucleotide sequence may be used for genetic manipulation to “humanize” the antibody or to improve the affinity, or other characteristics of the antibody. For example, the constant region may be engineered to more nearly resemble human constant regions to avoid immune response if the antibody is used in clinical trials and treatments in humans. It may be desirable to genetically manipulate the antibody sequence to obtain greater affinity to CD70 and greater efficacy in inhibiting CD70.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen binding site derived from a non-human immunoglobulin have been described, including chimeric antibodies having rodent or modified rodent V regions and their associated CDRs fused to human constant regions. See, for example, Winter et al. Nature 349:293-299, 1991, Lobuglio et al. Proc. Nat. Acad. Sci. USA 86:4220-4224, 1989, Shaw et al. J Immunol. 138:4534-4538, 1987, and Brown et al. Cancer Res. 47:3577-3583, 1987. Other references describe rodent CDRs grafted into a human supporting framework region (FR) prior to fusion with an appropriate human antibody constant region. See, for example, Riechmann et al. Nature 332:323-327, 1988, Verhoeyen et al. Science 239:1534-1536, 1988, and Jones et al. Nature 321:522-525, 1986. Another reference describes rodent CDRs supported by recombinantly engineered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g. PCT Publication No. PCT/GB99/01441; UK Patent Application No. 9809951.8. Other methods of humanizing antibodies that may also be utilized are disclosed by Daugherty et al., Nucl. Acids Res. 19:2471-2476, 1991, and in U.S. Pat. Nos. 6, 180,377; 6,054,297; 5,997,867; 5,866,692; 6,210,671; and 6,350,861; and in PCT Publication No. WO 01/27160.

The general principles related to humanized antibodies discussed above are also applicable to customizing antibodies for use, for example, in dogs, cats, primate, equines and bovines. Further, one or more aspects of humanizing an antibody described herein may be combined, e.g., CDR grafting, framework mutation and CDR mutation.

In one variation, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, CA) and HuMAb-Mouse® and TC Mouse™ from Medarex, Inc. (Princeton, NJ).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; and 6,265,150; and Winter et al., Annu. Rev. Immunol. 12:433-455, 1994. Alternatively, the phage display technology (McCafferty et al., Nature 348:552-553, 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3:564-571, 1993. Several sources of V-gene segments can be used for phage display. Clackson et al., Nature 352:624-628, 1991, isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597, 1991, or Griffith et al., EMBO J. 12:725-734, 1993. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” (Marks et al., Bio/Technol. 10:779-783, 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266, 1993. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756, 2001; Lonberg, N. and D. Huszar Int. Rev. Immunol 13:65, 1995; and Pollock, et al., J Immunol Methods 231:147, 1999. Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for CD70, or tumor antigens of interest.

The antibodies as described herein can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses, and magnetite. The nature of the carrier can be either soluble or insoluble for purposes of the invention. Those skilled in the art will know of other suitable carriers for binding antibodies, or will be able to ascertain such, using routine experimentation. In some embodiments, the carrier comprises a moiety that targets the myocardium.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO87/04462. The DNA also may be modified, for example, by substituting the coding sequence for human heavy and light chain constant regions in place of the homologous murine sequences, Morrison et al., Proc. Nat. Acad. Sci. 81:6851, 1984, or by covalently joining to the immunoglobulin coding sequence all or part of the coding sequence for a non-immunoglobulin polypeptide. In that manner, “chimeric” or “hybrid” antibodies are prepared that have the binding specificity of a monoclonal antibody herein.

The CD70 antibodies as described herein can be identified or characterized using methods known in the art, whereby reduction of CD70 expression levels are detected and/or measured. In some embodiments, an CD70 antibody is identified by incubating a candidate agent with CD70 and monitoring binding and/or attendant reduction of CD70 expression levels. The binding assay may be performed with purified CD70 polypeptide(s), or with cells naturally expressing, or transfected to express, CD70 polypeptide(s). In one embodiment, the binding assay is a competitive binding assay, where the ability of a candidate antibody to compete with a known CD70 antibody for CD70 binding is evaluated. The assay may be performed in various formats, including the ELISA format.

Following initial identification, the activity of a candidate CD70 antibody can be further confirmed and refined by bioassays, known to test the targeted biological activities. Alternatively, bioassays can be used to screen candidates directly. Some of the methods for identifying and characterizing antibodies are described in detail in the Examples.

CD70 antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In an additional example, epitope mapping can be used to determine the sequence to which an antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an CD70 or other tumor antigen antibody. In another example, the epitope to which the CD70 antibody binds can be determined in a systematic screening by using overlapping peptides derived from the CD70 sequence and determining binding by the CD70 antibody. According to the gene fragment expression assays, the open reading frame encoding CD70 is fragmented either randomly or by specific genetic constructions and the reactivity of the expressed fragments of CD70 with the antibody to be tested is determined. The gene fragments may, for example, be produced by PCR and then transcribed and translated into protein in vitro, in the presence of radioactive amino acids. The binding of the antibody to the radioactively labeled CD70 is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant CD70 in which various fragments of the CD70 protein have been replaced (swapped) with sequences from CD70 from another species (e.g., mouse), or a closely related, but antigenically distinct protein. By assessing binding of the antibody to the mutant CD70, the importance of the particular CD70 fragment to antibody binding can be assessed. In the case of CD70 specific antibody (i.e. antibody that does not bind CD70wt (wild type) or any other proteins), epitope can be deduced from the sequence alignment of CD70 to CD70wt.

Yet another method which can be used to characterize an CD70 antibody is to use competition assays with other antibodies known to bind to the same antigen, i.e., various fragments on CD70, to determine if the CD70 antibody binds to the same epitope as other antibodies. Competition assays are well known to those of skill in the art.

An expression vector can be used to direct expression of an CD70 antibody. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventrical, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J.A. Wolff, ed., 1994; Wu et al., J. Biol. Chem., 263:621, 1988; Wu et al., J. Biol. Chem., 269:542, 1994; Zenke et al., Proc. Natl. Acad. Sci. USA, 87:3655, 1990; and Wu et al., J. Biol. Chem., 266:338, 1991. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1:51, 1994; Kimura, Human Gene Therapy, 5:845, 1994; Connelly, Human Gene Therapy, 1995, 1:185; and Kaplitt, Nature Genetics, 6:148, 1994). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5, 219,740 and 4,777,127; GB Pat. No. 2,200,651; and EP Pat. No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 3:147, 1992); ligand-linked DNA (see, e.g., Wu, J. Biol. Chem., 264:16985, 1989); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 14:2411, 1994 and in Woffendin, Proc. Natl. Acad. Sci., 91:1581, 1994.

In some embodiments, the invention encompasses compositions, including pharmaceutical compositions, comprising antibodies described herein or made by the methods and having the characteristics described herein. As used herein, compositions comprise one or more antibodies that bind to CD70, and/or one or more polynucleotides comprising sequences encoding one or more these antibodies. These compositions may further comprise suitable excipients, such as pharmaceutically acceptable excipients including buffers, which are well known in the art.

The invention also provides methods of making any of these antibodies. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In another alternative, the antibodies can be made recombinantly using procedures that are well known in the art. In one embodiment, a polynucleotide comprises a sequence encoding the heavy chain and/or the light chain variable regions of antibody 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02 or P16C05. The sequence encoding the antibody of interest may be maintained in a vector in a host cell and the host cell can then be expanded and frozen for future use. Vectors (including expression vectors) and host cells are further described herein.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

In the recombinant humanized antibodies, the Fcγ portion can be modified to avoid interaction with Fcγ receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, for example, U.S. Pat. Nos. 5,997,867 and 5,866,692.

The invention encompasses modifications to the antibodies and polypeptides of the invention including variants shown in Table 5, including functionally equivalent antibodies which do not significantly affect their properties and variants which have enhanced or decreased activity and/or affinity. For example, the amino acid sequence may be mutated to obtain an antibody with the desired binding affinity to CD70. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino-and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 5 under the heading of “conservative substitutions.” If such substitutions result in a change in biological activity, then more substantial changes, denominated “exemplary substitutions” in Table 5, or as further described below in reference to amino acid classes, may be introduced and the products screened. In some embodiments, substitution variants of antibodies provided herein have no more than 15, 14, 13, 12, 11, 10, 9, 8, 7, 6, 5, 4, 3, 2, or 1 conservative substitution in the VH or VL region as compared to the reference parent antibody. In some embodiments, the substitutions are not within a CDR of the VH or VL region.

TABLE 5
Amino Acid Substitutions

| Original Residue | | |
|---|---|---|
| (naturally | | |
| occurring amino | Conservative | |
| acid) | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring amino acid residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, Chem. Immunol. 65:111-128, 1997; Wright and Morrison, TibTECH 15:26-32, 1997). The oligosaccharide side chains of the immunoglobulins affect the protein's function (Boyd et al., Mol. Immunol. 32:1311-1318, 1996; Wittwe and Howard, Biochem. 29:4175-4180, 1990) and the intramolecular interaction between portions of the glycoprotein, which can affect the conformation and presented three-dimensional surface of the glycoprotein (Jefferis and Lund, supra; Wyss and Wagner, Current Opin. Biotech. 7:409-416, 1996). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180, 1999).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., J. Biol. Chem. 272:9062-9070, 1997).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides.

These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

The invention includes affinity matured embodiments. For example, affinity matured antibodies can be produced by procedures known in the art (Marks et al., Bio/Technology, 10:779-783, 1992; Barbas et al., Proc Nat. Acad. Sci, USA 91:3809-3813, 1994; Schier et al., Gene, 169:147-155, 1995; Yelton et al., J. Immunol., 155:1994-2004, 1995; Jackson et al., J. Immunol., 154 (7): 3310-9, 1995, Hawkins et al., J. Mol. Biol., 226:889-896, 1992; and PCT Publication No. WO2004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using Biacore™ surface plasmon resonance analysis, which detects differences in binding affinity of about 2-fold or greater. Biacore™ is particularly useful when the starting antibody already binds with a relatively high affinity, for example a K_{D} of about 10 nM or lower. Screening using Biacore™ surface plasmon resonance is described in the Examples, herein.

Binding affinity may be determined using Kinexa Biocensor, scintillation proximity assays, ELISA, ORIGEN immunoassay (IGEN), fluorescence quenching, fluorescence transfer, and/or yeast display. Binding affinity may also be screened using a suitable bioassay.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., Gene 137(1):109-18, 1993.

The CDR may be CDRH3 and/or CDRL3. The CDR may be one or more of CDRL1, CDRL2, CDRL3, CDRH1, CDRH2, and/or CDRH3. The CDR may be a Kabat CDR, a Chothia CDR, or an extended CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using Biacore™ surface plasmon resonance analysis, and selection using any method known in the art for selection, including phage display, yeast display, and ribosome display.

The invention also encompasses fusion proteins comprising one or more fragments or regions from the antibodies of this invention. In one embodiment, a fusion polypeptide is provided that comprises at least 10 contiguous amino acids of the variable light chain region shown in SEQ ID NOs: 1, 3, 5, 7, 9, 11, 13, 15, 17, 19, 21, 23, 25, 27, 29, 31, 33, 35, 37, 39, 41, 43, 45 or 47, and/or at least 10 amino acids of the variable heavy chain region shown in SEQ ID NOs: 2, 4, 6, 8, 10, 12, 14, 16, 18, 20, 22, 24, 26, 28, 30, 32, 34, 36, 38, 40, 42, 44, 46 or 48. In other embodiments, a fusion polypeptide is provided that comprises at least about 10, at least about 15, at least about 20, at least about 25, or at least about 30 contiguous amino acids of the variable light chain region and/or at least about 10, at least about 15, at least about 20, at least about 25, or at least about 30 contiguous amino acids of the variable heavy chain region. In another embodiment, the fusion polypeptide comprises one or more CDR(s). In still other embodiments, the fusion polypeptide comprises CDR H3 (VH CDR3) and/or CDR L3 (VL CDR3). For purposes of this invention, a fusion protein contains one or more antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag (SEQ ID NO: 531). Tags are well known in the art.

A fusion polypeptide can be created by methods known in the art, for example, synthetically or recombinantly. Typically, the fusion proteins of this invention are made by preparing an expressing a polynucleotide encoding them using recombinant methods described herein, although they may also be prepared by other means known in the art, including, for example, chemical synthesis.

This invention also provides compositions comprising antibodies conjugated (for example, linked) to an agent that facilitate coupling to a solid support (such as biotin or avidin). For simplicity, reference will be made generally to antibodies with the understanding that these methods apply to any of the CD70 antibody embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The invention also provides isolated polynucleotides encoding the antibodies of the invention, and vectors and host cells comprising the polynucleotide.

Accordingly, the invention provides polynucleotides (or compositions, including pharmaceutical compositions), comprising polynucleotides encoding any of the following: 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02 or P16C05, or any fragment or part thereof having the ability to bind CD70.

In another aspect, the invention provides polynucleotides encoding any of the antibodies (including antibody fragments) and polypeptides described herein, such as antibodies and polypeptides having impaired effector function. Polynucleotides can be made and expressed by procedures known in the art.

In another aspect, the invention provides compositions (such as a pharmaceutical compositions) comprising any of the polynucleotides of the invention. In some embodiments, the composition comprises an expression vector comprising a polynucleotide encoding any of the antibodies described herein.

Expression vectors, and administration of polynucleotide compositions are further described herein.

In another aspect, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M.O., 1978, A model of evolutionary change in proteins—Matrices for detecting distant relationships. In Dayhoff, M.O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D.G. and Sharp, P.M., 1989, CABIOS 5:151-153; Myers, E.W. and Muller W., 1988, CABIOS 4:11-17; Robinson, E.D., 1971, Comb. Theor. 11:105; Santou, N., Nes, M., 1987, Mol. Biol. Evol. 4:406-425; Sneath, P.H.A. and Sokal, R.R., 1973, Numerical Taxonomy the Principles and Practice of Numerical Taxonomy, Freeman Press, San Francisco, CA; Wilbur, W.J. and Lipman, D.J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42°° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Patent Nos. 4,683, 195, 4,800, 159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to CD70 is effected by an immunoassay or FACS. A cell overexpressing the antibody or protein of interest can be identified.

### Methods of Using the CD70 Antibodies

The antibodies of the present invention are useful in various applications including, but are not limited to, therapeutic treatment methods and diagnostic treatment methods.

The antibodies (e.g., monospecific and bispecific) obtained by the methods described above can be used as a medicament. In some embodiments, such a medicament can be used for treating cancer. In some embodiments, the cancer is a cancer of hematopoietic origin, such as a lymphoma or leukemia. In some embodiments, the cancer is Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer

In some embodiments, provided is a method of inhibiting tumor growth or progression in a subject who has malignant cells expressing CD70, comprising administering to the subject in need thereof an effective amount of a composition comprising the CD70 antibodies (e.g., CD70-CD3 bispecific antibodies) as described herein. In other embodiments, provided is a method of inhibiting metastasis of cells expressing CD70 in a subject, comprising administering to the subject in need thereof an effective amount of a composition comprising the CD70 antibodies (e.g., CD70-CD3 bispecific antibodies) as described herein. In other embodiments, provided is a method of inducing tumor regression in malignant cells in a subject, comprising administering to the subject in need thereof an effective amount of a composition comprising the CD70 antibodies (e.g., CD70-CD3 bispecific antibodies) as described herein.

In some embodiments, the antibody (e.g., CD70-CD3 bispecific antibody) according to the invention can be used in the manufacture of a medicament for treatment of a cancer in a patient in need thereof.

In some embodiments, the treatment can be in combination with one or more therapies against cancer selected from the group of antibodies therapy, chemotherapy, cytokines therapy, targeted therapy, vaccine therapy, dendritic cell therapy, gene therapy, hormone therapy, surgical resection, laser light therapy and radiation therapy.

For example, in some embodiments, the CD70 antibodies (e.g., CD70-CD3 bispecific antibodies) of the present invention are administered to a patient in conjunction with (e.g., before, simultaneously or following) treatment with small molecule Tyrosine Kinase Inhibitors (TKIs) such as sunitinib and pazopanib that target Vascular Endothelial Growth Factor (VEGF) receptor, monoclonal antibody targeting VEGF such as bevacizumab, mammalian target of Rapamycin (mTOR) inhibitor temsirolimus, as well as high dose IL-2. In some embodiments, the CD70 antibodies (e.g., CD70-CD3 bispecific antibodies) of the present invention are administered to a patient in conjunction with one or more of the following: an anti-PD-1 antibody (e.g., nivolumab, pembrolizumab, or PF-06801591), an anti-PD-L1 antibody (e.g., avelumab, atezolizumab, or durvalumab) an anti-OX40 antibody (e.g., PF-04518600), an anti-4-1BB antibody (e.g., PF-05082566), an anti-MCSF antibody (e.g., PD-0360324), an anti-GITR antibody, and/or an anti-TIGIT antibody.

The administration of the antibodies (e.g., monospecific or bispecific) according to the invention may be carried out in any convenient manner, including by aerosol inhalation, injection, ingestion, transfusion, implantation or transplantation. The compositions described herein may be administered to a patient subcutaneously, intradermally, intratumorally, intracranially, intranodally, intramedullary, intramuscularly, by intravenous or intralymphatic injection, or intraperitoneally. In one embodiment, the antibody compositions of the invention are preferably administered by intravenous injection.

In some embodiments, the administration of the antibodies (e.g., monospecific or bispecific) can comprise administration of, for example, about 0.01 to about 20 mg per kg body weight including all integer values of mg per kg within those ranges. In some embodiments, the administration of the antibodies can comprise administration of about 0.1 to 10 mg per kg body weight including all integer values of mg per kg within those ranges. The antibody can be administrated in one or more doses. In some embodiments, said effective amount of the antibody can be administrated as a single dose. In some embodiments, said effective amount of antibodies can be administrated as more than one dose over a period time. Timing of administration is within the judgment of managing physician and depends on the clinical condition of the patient. While individual needs vary, determination of optimal ranges of effective amounts of a given antibody (e.g., monospecific or bispecific) for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of heteromultimeric antibody or composition comprising those antibodies are administrated parenterally. In some embodiments, administration can be an intravenous administration. In some embodiments, administration can be directly done by injection within a tumor.

In some embodiments, anti-CD70 antibodies provided herein may be used for diagnostic purposes, such in assays to identify CD70 protein in samples (e.g. in immunohistochemistry assays) or in patients.

### Compositions

In one aspect, the present invention provides a pharmaceutical composition comprising an antibody (e.g., monospecific or bispecific) of the invention or portion thereof as described above in a pharmaceutically acceptable carrier. In certain embodiments, the polypeptides of the invention may be present in a neutral form (including zwitter ionic forms) or as a positively or negatively-charged species. In some embodiments, the polypeptides may be complexed with a counterion to form a “pharmaceutically acceptable salt,” which refers to a complex comprising one or more polypeptides and one or more counterions, where the counterions are derived from pharmaceutically acceptable inorganic and organic acids and bases.

The antibody (e.g., monospecific or bispecific) or portions thereof, may be administered alone or in combination with one or more other polypeptides of the invention or in combination with one or more other drugs (or as any combination thereof). The pharmaceutical compositions, methods and uses of the invention thus also encompass embodiments of combinations (co-administration) with other active agents, as detailed below.

As used herein, the terms “co-administration,” “co-administered” and “in combination with,” referring to the antibodies of the invention and one or more other therapeutic agents, is intended to mean, and does refer to and include the following: (i) simultaneous administration of such combination of an antibody disclosed herein and therapeutic agent(s) to a patient in need of treatment, when such components are formulated together into a single dosage form which releases said components at substantially the same time to said patient; (ii) substantially simultaneous administration of such combination of an antibody disclosed herein and therapeutic agent(s) to a patient in need of treatment, when such components are formulated apart from each other into separate dosage forms which are taken at substantially the same time by said patient, whereupon said components are released at substantially the same time to said patient; (iii) sequential administration of such combination of an antibody disclosed herein and therapeutic agent(s) to a patient in need of treatment, when such components are formulated apart from each other into separate dosage forms which are taken at consecutive times by said patient with a significant time interval between each administration, whereupon said components are released at substantially different times to said patient; and (iv) sequential administration of such combination of an antibody disclosed herein and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

Generally, the antibody (e.g., monospecific or bispecific) disclosed herein or portions thereof are suitable to be administered as a formulation in association with one or more pharmaceutically acceptable excipient(s). The term ‘excipient’ is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP conditions.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage. Any method for administering peptides, proteins or antibodies accepted in the art may suitably be employed for the heterodimeric proteins and portions thereof disclosed herein.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. Preferred embodiments include the intravenous and the subcutaneous routes.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include controlled, delayed, sustained, pulsed, targeted and programmed release formulations. For example, in one aspect, sterile injectable solutions can be prepared by incorporating the heterodimeric protein, e.g., bispecific antibody, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

Generally, for administration of the antibodies described herein (monospecific or bispecific), the candidate dosage can be administered daily, every week, every other week, every three weeks, every four weeks, every five weeks, every six weeks, every seven weeks, every eight weeks, every ten weeks, every twelve weeks, or more than every twelve weeks. For repeated administrations over several days or longer, depending on the condition, the treatment is sustained until a desired suppression of symptoms occurs or until sufficient therapeutic levels are achieved, for example, to reduce symptoms associated with cancer. The progress of this therapy is easily monitored by conventional techniques and assays. The dosing regimen (including the anti-FLT monospecific or bispecific antibody used) can vary over time.

In some embodiments, the candidate dosage is administered daily with the dosage ranging from about any of 1 μg/kg to 30 μg/kg to 300 μg/kg to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg to 30 μg/kg to 300 μg/kg to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg to 30 μg/kg to 300 μg/kg to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg to 30 μg/kg to 300 μg/kg to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In some embodiments, the candidate dosage is administered every month or every four weeks with the dosage ranging from about any of 1 μg/kg to 30 μg/kg to 300 μg/kg to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used.

### Kits

The invention also provides kits for use in the instant methods. Kits of the invention include one or more containers comprising the antibody (e.g., monospecific or bispecific) as described herein and instructions for use in accordance with any of the methods of the invention described herein. Generally, these instructions comprise a description of administration of the antibody protein for the above described therapeutic treatments.

The instructions relating to the use of the antibody (e.g., monospecific or bispecific) as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a bispecific antibody. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1: Determination of Kinetics and Affinity of Human CD70/CD70 Antibodies Interactions at 37° C.

The kinetics and affinity of anti-CD70 antibodies disclosed herein can be measured on a Biacore T200 surface Plasmon resonance biosensor (GE Lifesciences, Piscataway NJ).

### Example 2: T-Cell Mediated Killing of RCC Cell Lines Using CD70-CD3 Bispecific IgG in Vitro

Human anti-CD70 and human anti-CD3 (h2B4-VH-hnps VL-TK (“H2B4”)) antibodies are expressed as human IgG2dA_D265A engineered with EEEE on one arm and RRRR on the other arm for bispecific exchange at positions 223, 225, and 228 (e.g., (C223E or C223R), (E225E or E225R), and (P228E or P228R)) in the hinge region and at position 409 or 368 (e.g., K409R or L368E (EU numbering scheme)) in the CH3 region of human IgG2 (SEQ ID NO: 279). The CD70/CD3 bispecific antibody also has the mutation from D to A at position 265 (EU numbering scheme).

CD3+ T cells from human PBMC are negatively selected using Pan T Cell Isolation kit, human (Miltenyi, San Diego CA). Target expressing (786-O) cells and CD3+ T-cells are seeded on clear U-bottom plates at 20000 and 100000 cells/well respectively. Cells are treated with 8-fold serially diluted bispecific antibody. RCC cell depletion is determined by flow-cytometry analysis 24 hours after treatment. Cell depletion is measured by contrast to control treated cells. EC50 is calculated by Prism software.

### Example 3: CD70-CD3 Bispecific IgG Induces Tumor Ablation in RCC Subcutaneous Xenograft Model

NOD scid gamma (NSG) mice are implanted with 786-O tumors subcutaneously and once the tumors attained a volume of 200 mm³, the mice are dosed with 20 million expanded T cells each intraperitoneally. Two days later the anti CD70 bispecific antibodies are dosed at 300, 100, or 30 ug/mL intravenously via tail vein injection to determine the optimal bispecific antibody dose.

### Materials and Methods:

NOD scid gamma (NSG) mice are shaved and prepared for subcutaneous tumor implant on the right flank. 786-O tumor cells that are known to express CD70 are expanded in RPMI supplemented with 10% FBS. On Day 0, 786-O cells are resuspended in serum-free RPMI at the required concentration to inject 5 million cells per animal. Tumor cells are injected in 100 uL of serum-free RPMI combined with 100 uL Matrigel (Corning) per animal subcutaneously. Day 0 baseline body weights are recorded for all animals immediately after tumor implant. Tumors are measured twice a week starting on Day 9 using Digimatic Calipers (Mitutoyo) and body weights recorded. On Day 14, when the tumors attained 200 mm³ (standard error 8.39) 40 tumor-bearing mice are randomized to 4 groups of 10 mice each. T cells are thawed and expanded and then resuspended in serum-free RPMI at the required concentration to inject 20 million T cells per animal. T cells are injected in 200 uL of serum-free RPMI per animal intraperitoneally. Two days later bispecifics are dosed intravenously via tail vein at 300, 100, or 30 ug/mL per animal. Tumors are measured and body weights recorded twice a week till the untreated group reached the study end-point (1500 mm³ tumor volume).

Tumor volumes (mean and error SEM) are plotted on GraphPad Prism and statistics are calculated using one-way ANOVA with repeated measures.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1-23. (canceled)

24. An isolated antibody, which specifically binds to Cluster of Differentiation 70 (CD70), wherein the antibody comprises a heavy chain variable (VH) region comprising a VH complementarity determining region one (CDR1), a VH CDR2, and a VH CDR3, and a light chain variable (VL) region comprising a VL CDR1, a VL CDR2, and a VL CDR3, wherein:
the VH CDR1 comprises the amino acid sequence of SEQ ID NO: 332,
the VH CDR2 comprises the amino acid sequence of SEQ ID NO: 335,
the VH CDR3 comprises the amino acid sequence of SEQ ID NO: 337; or
the VH CDR 1 comprises the amino acid sequence of SEQ ID NO: 333,
the VH CDR2 comprises the amino acid sequence of SEQ ID NO: 336,
the VH CDR3 comprises the amino acid sequence of SEQ ID NO: 337; or
the VH CDR 1 comprises the amino acid sequence of SEQ ID NO: 334,
the VH CDR2 comprises the amino acid sequence of SEQ ID NO: 335,
the VH CDR3 comprises the amino acid sequence of SEQ ID NO: 337; and
wherein
the VL CDR1 comprises the amino acid sequence of SEQ ID NO: 464,
the VL CDR2 comprises the amino acid sequence of SEQ ID NO: 465, and
the VL CDR3 comprises the amino acid sequence of SEQ ID NO: 466.

25. The antibody of claim 24, wherein the VH region comprises the amino acid sequence of SEQ ID NO: 289 and the VL region comprises the amino acid sequence of SEQ ID NO: 288.

26. A bispecific antibody comprising a first antigen binding site of the bispecific antibody specifically binding to CD70, and comprising a second antigen binding site of the bispecific antibody capable of recruiting the activity of a human immune effector cell by specifically binding to an effector antigen located on the human immune effector cell, wherein the first antigen binding site comprises
a heavy chain variable (VH) region comprising (i) a VH complementarity determining region one (CDR1), a VH CDR2, and a VH CDR3, and a light chain variable (VL) region comprising a VL CDR1, a VL CDR2, and a VL CDR3, wherein:
the VH CDR1 comprises the amino acid sequence of SEQ ID NO: 332,
the VH CDR2 comprises the amino acid sequence of SEQ ID NO: 335,
the VH CDR3 comprises the amino acid sequence of SEQ ID NO: 337; or
the VH CDR 1 comprises the amino acid sequence of SEQ ID NO: 333,
the VH CDR2 comprises the amino acid sequence of SEQ ID NO: 336,
the VH CDR3 comprises the amino acid sequence of SEQ ID NO: 337; or
the VH CDR 1 comprises the amino acid sequence of SEQ ID NO: 334,
the VH CDR2 comprises the amino acid sequence of SEQ ID NO: 335,
the VH CDR3 comprises the amino acid sequence of SEQ ID NO: 337; and
wherein
the VL CDR1 comprises the amino acid sequence of SEQ ID NO: 464,
the VL CDR2 comprises the amino acid sequence of SEQ ID NO: 465, and
the VL CDR3 comprises the amino acid sequence of SEQ ID NO: 466.

27. The bispecific antibody of claim 26, wherein the effector antigen is CD3.

28. The bispecific antibody of claim 26, wherein the second antigen binding site comprises
a) a heavy chain variable (VH) region comprising (i) a VH complementary determining region one (CDR1) comprising the sequence shown in SEQ ID NO: 267, 268, or 269; (ii) a VH CDR2 comprising the sequence shown in SEQ ID NO: 270 or 271; and (iii) a VH CDR3 comprising the sequence shown in SEQ ID NO: 272; and
b) a light chain variable (VL) region comprising (i) a VL CDR1 comprising the sequence shown in SEQ ID NO: 273; (ii) a VL CDR2 comprising the sequence shown in SEQ ID NO: 274; and (iii) a VL CDR3 comprising the sequence shown in SEQ ID NO: 275.

29. The bispecific antibody of claim 26, wherein both the first and the second antigen binding sites of the bispecific antibody comprise amino acid modifications at EU numbering scheme positions 223, 225, and 228 in the hinge region and at EU numbering scheme position 409 or 368 in the CH3 region of a human IgG2 having the sequence of SEQ ID NO: 279.

30. The bispecific antibody of claim 29, further comprising an amino acid modification at one or more of EU numbering scheme positions 265, 330 and 331 of the human IgG2.

31. A nucleic acid encoding the antibody of claim 24.

32. A vector comprising the nucleic acid of claim 31.

33. A host cell comprising the nucleic acid of claim 31.

34. A pharmaceutical composition comprising the antibody of claim 24.

35. A method of treating a CD70 associated disease comprising administering to a subject in need thereof an effective amount of the antibody of claim 24.

36. The method of claim 35, wherein the condition is a cancer.

37. The method of claim 36, wherein the cancer is selected from the group consisting of renal cell carcinoma, clear cell renal cell carcinoma, glioblastoma, glioma, non-Hodgkin's lymphoma (NHL), Hodgkin's disease (HD), Waldenstrom's macroglobulinemia, acute myeloid leukemia, multiple myeloma, diffuse large-cell lymphoma, follicular lymphoma or non-small cell lung cancer.

38. A method of treating a CD70 associated disease comprising administering to a subject in need thereof an effective amount of the pharmaceutical composition of claim 34 to the subject.

39. A method of producing an antibody, comprising culturing the host cell of claim 33 under conditions that result in production of the antibody, and isolating the antibody from the host cell or culture.

40. The antibody of claim 24, wherein:
the VH CDR1 comprises the amino acid sequence of SEQ ID NO: 332,
the VH CDR2 comprises the amino acid sequence of SEQ ID NO: 335,
the VH CDR3 comprises the amino acid sequence of SEQ ID NO: 337.

41. An isolated antibody, which specifically binds to Cluster of Differentiation 70 (CD70), wherein the antibody comprises a heavy chain variable (VH) region comprising a VH complementarity determining region one (CDR1), a VH CDR2, and a VH CDR3, and a light chain variable (VL) region comprising a VL CDR1, a VL CDR2, and a VL CDR3, wherein the VL region comprises the amino acid sequence of SEQ ID NO: 288 and the VH region comprises the amino acid sequence of SEQ ID NO: 289.

42. A pharmaceutical composition comprising the antibody of claim 40.

43. A pharmaceutical composition comprising the antibody of claim 41.
